(19) United States
(12) Patent Application Publication
(10) Pub. No.: US 2009/0075978 A1
(43) Pub. Date: Mar. 19, 2009

(54) SUBSTITUTED PROPIOLIC ACID AMIDES AND THEIR USE FOR PRODUCING DRUGS

(75) Inventors: Michael HAURAND, Aachen (DE); Klaus SCHIENE, Juchen (DE); Sven KUHNERT, Duren (DE); Melanie REICH, Aachen (DE); Saskia ZEMOLKA, Aachen (DE)

Correspondence Address: NORRIS, MCLAUGHLIN & MARCUS, PA, 875 THIRD AVENUE, 18TH FLOOR, NEW YORK, NY, 10022, US

(73) Assignee: Grunenthal GmbH, Aachen (DE)

(21) Appl. No.: 12/146,814

(22) Filed: Jun. 26, 2008

Related U.S. Application Data
(63) Continuation of application No. PCT/EP2006/012479, filed on Dec. 22, 2006.

(30) Foreign Application Priority Data
Dec. 28, 2005 (DE) 10 2005 062 987.3

(51) Int. Cl. A61K 31/472; C07D 217/04; C07D 401/06; C07D 498/04; C07D 487/04; A61K 31/4035; A61K 31/4375; A61K 31/55; A61P 25/00; A61P 1/00; A61P 11/00; A61K 31/4985; A61K 31/437; A61K 31/404; A61K 31/47; C07D 223/16; C07D 215/00; C07D 471/04

(52) U.S. Cl. 514/213.01; 546/146; 546/115; 544/350; 540/593; 546/165; 546/122; 514/307; 514/311; 514/416; 514/419; 514/300; 514/302; 514/249

(57) ABSTRACT

The present invention relates to substituted propiolic acid amides, to methods for the production thereof, to medicaments containing these compounds and to the use thereof for producing medicaments.

The present invention relates to substituted propiolic acid amides, to methods for the production thereof, to medicaments containing these compounds and to the use thereof for producing medicaments.

Pain is one of the basic clinical symptoms. There is a worldwide need for effective pain treatments. The urgency of the requirement for therapeutic methods for providing tailored and targeted treatment of chronic and non-chronic pain, this being taken to mean pain treatment which is effective and satisfactory from the patient's standpoint, is also evident from the large number of scientific papers relating to applied analgesia and to basic nociception research which have appeared in recent times.

Conventional opioids, such as for example morphine, are effective in the treatment of severe to very severe pain, but they often lead to unwanted accompanying symptoms, such as for example respiratory depression, vomiting, sedation, constipation or the development of tolerance. Moreover, they are frequently insufficiently effective in the case of neuropathic pain, suffered in particular by tumour patients.

One object of the present invention was accordingly to provide novel compounds which are suitable in particular as pharmaceutical active ingredients in medicaments, preferably in medicaments for the treatment of pain.

It has now surprisingly been found that the substituted propiolic acid amides of the general formula I stated hereinafter are suitable for mGluR5 receptor regulation and may therefore be used in particular as pharmaceutical active ingredients in medicaments for the prevention and/or treatment of disorders or diseases associated with these receptors or processes.

The present invention accordingly provides substituted propiolic acid amides of the general formula I

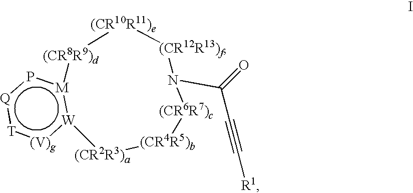

in which
R¹ denotes unsubstituted or substituted aryl or unsubstituted or substituted heteroaryl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R³, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
or R² and R³ or R⁴ and R⁵ or R⁶ and R⁷ or R³ and R⁹ or R¹⁰ and R¹¹ or R¹² and R¹³ in each case together denote an oxo group (═O) or a thioxo group (═S);
M and W, mutually independently, in each case denote N or C;
P denotes CR¹⁴, N, NR¹⁵, O or S;
Q denotes CR¹⁶, N, NR¹⁷, O or S;
T denotes CR¹⁸, N, NR¹⁹, O or S;
V denotes CR²⁰, N, NR²¹, O or S;
g denotes 0 or 1:
wherein M, W; P, Q, T, V and g together form a 5- or 6-membered aromatic or heteroaromatic ring;
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴—O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷—NH—C(═O)—NH—R³⁸; —NH—C(═S)—NH—R³⁹; —NH—S(═O)₂—R⁴⁰; —NR⁴¹—S(═O)₂—R⁴²; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
R¹⁵, R¹⁷, R¹⁹ and R²¹, mutually independently, in each case denote H; —C(═O)—OH; —C(═O)—H; —C(═O)—R²⁷; —C(═O)—O—R²⁸; —C(═O)—NH₂; —C(═O)—NH—R³³; —C(═O)—NR³⁴R³⁵; —S(═O)—R³⁶; —S(═O)₂—R³⁷; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
and R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴², mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
with the proviso that compounds are excepted in which the sum of a, b, c, d, e and f is equal to 3, W and M in each case denote C, P denotes CR¹⁴, Q denotes CR¹⁶, T denotes CR¹⁸ and V denotes CR²⁰;
with the proviso that compounds are excepted in which g is 0, W and M in each case denote C, P denotes NR¹⁵, Q denotes CR¹⁶ and T denotes N;
and with the proviso that compounds are excepted in which g is 0, W and M in each case denote C, P denotes N, Q denotes CR¹⁶ and T denotes NR¹⁹; in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

For the purposes of the present invention, the term “alkyl” covers acyclic saturated hydrocarbon residues, which may be branched or straight-chain and unsubstituted or at least monosubstituted with, as in the case of C₁₋₁₂ alkyl, 1 to 12 (i.e. 1, 2, 3, 4, 5, 6, 7, 8, 9, 10, 11 or 12) C atoms or with, as in the case of C₁₋₆ alkyl, 1 to 6 (i.e. 1, 2, 3, 4, 5 or 6) C atoms. If one or more of the substituents denote an alkyl residue or comprise an alkyl residue which is mono- or polysubstituted, this may preferably be substituted with optionally 1, 2, 3, 4 or 5, particularly preferably with 1, 2 or 3, substituents mutually independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the above-stated-C₁₋₅ alkyl residues may in each case be linear or branched and the above-stated phenyl residues may preferably be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl. Particularly preferred substituents may be selected mutually independently from the group consisting of F, Cl, Br, I, —NO₂, —CN, —OH, —SH, —NH₂, —N(CH₃)₂, —N(C₂H₅)₂ and —N(CH₃)(C₂H₅).

Examples which may be mentioned of suitable C₁₋₁₂ alkyl residues, which may be unsubstituted or mono- or polysubstituted, are methyl, ethyl, n-propyl, isopropyl, n-butyl, isobutyl, 2-butyl, tert.-butyl, n-pentyl, 2-pentyl, 3-pentyl, iso-pentyl, neopentyl, n-hexyl, 2-hexyl, 3-hexyl, n-heptyl, n-octyl, —C(H)(C₂H₅)₂, —C(H)(n-C₃H₇)₂ and —CH₂—CH₂—C(H)(CH₃)—(CH₂)₃—CH₃. Examples which may be mentioned of suitable C₁₋₆ alkyl residues are methyl, ethyl, n-propyl, isopropyl, n-butyl, isobutyl, 2-butyl, tert.-butyl, n-pentyl, 2-pentyl, 3-pentyl, iso-pentyl, neopentyl, n-hexyl, 2-hexyl and 3-hexyl.

Polysubstituted alkyl residues are understood to be those alkyl residues which are either poly-, preferably di- or trisubstituted, either on different or on the same C atoms, for example trisubstituted on the same C atom as in the case of —CF₃, or at different locations as in the case of —(CHCl)—(CH₂F). Polysubstitution may proceed with identical or different substituents. Examples which may be mentioned of suitable substituted alkyl residues are —CF₃, —CF₂H, —CFH₂, —(CH₂)—OH, —(CH₂)—NH₂, —(CH₂)—CN, —(CH₂)—(CF₃), —(CH₂)—(CHF₂), —(CH₂)—(CH₂F), —(CH₂)—(CH₂)—OH, —(CH₂)—(CH₂)—NH₂, —(CH₂)—(CH₂)—CN, —(CF₂)—(CF₃), —(CH₂)—(CH₂)—(—CF₃, and —(CH₂)—(CH₂)—(CH₂)—OH.

For the purposes of the present invention, the term “alkenyl” covers acyclic unsaturated hydrocarbon residues, which may be branched or straight-chain and unsubstituted or at least monosubstituted and comprise at least one double bond, preferably 1, 2 or 3 double bonds, with, as in the case of C₂₋₁₂ alkenyl, 2 to 12 (i.e. 2, 3, 4, 5, 6, 7, 8, 9, 10, 11 or 12) C atoms or with, as in the case of C₂₋₆ alkenyl, 2 to 6 (i.e. 2, 3, 4, 5 or 6) C atoms. If one or more of the substituents denote an alkenyl residue or comprise an alkenyl residue which is mono- or polysubstituted, this may preferably be substituted with optionally 1, 2, 3, 4 or 5, particularly preferably with 1, 2 or 3, substituents mutually independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the above-stated-C₁₋₅ alkyl residues may in each case be linear or branched and the above-stated phenyl residues may preferably be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl. Particularly preferred substituents may be selected mutually independently from the group consisting of F, Cl, Br, I, —NO₂, —CN, —OH, —SH, —NH₂, —N(CH₃)₂, —N(C₂H₅)₂ and —N(CH₃)(C₂H₅).

Examples which may be mentioned of suitable C₂₋₁₂ alkenyl residues are ethenyl, 1-propenyl, 2-propenyl, 1-butenyl, 2-butenyl, 3-butenyl, 1-pentenyl, 2-pentenyl, 3-pentenyl, 4-pentenyl, hexenyl, —CH═CH—CH═CH—CH₃ and —CH₂—CH₂—CH═CH₂.

Polysubstituted alkenyl residues are understood to be those alkenyl residues which are either poly-, preferably disubstituted, either on different or on the same C atoms, for example disubstituted on the same C atom as in the case of —CH═CCl₂, or at different locations as in the case of CCl═CH—(CH₂)—NH₂. Polysubstitution may proceed with identical or different substituents. Examples which may be mentioned of suitable substituted alkenyl residues are —CH═CH—(CH₂)—OH, —CH═CH—(CH₂)—NH₂ and —CH═CH—CN.

For the purposes of the present invention, the term “alkynyl” covers acyclic unsaturated hydrocarbon residues, which may be branched or straight-chain and unsubstituted or at least monosubstituted and comprise at least one triple bond, preferably 1 or 2 triple bonds, with, as in the case of C₂₋₁₂ alkynyl, 2 to 12 (i.e. 2, 3, 4, 5, 6, 7, 8, 9, 10, 11 or 12) C atoms or with, as in the case of C₂₋₆ alkynyl, 2 to 6 (i.e. 2, 3, 4, 5 or 6) C atoms. If one or more of the substituents denote an alkynyl residue or comprise an alkynyl residue which is mono- or polysubstituted, this may preferably be substituted with optionally 1, 2, 3, 4 or 5, particularly preferably with optionally 1 or 2, substituents mutually independently selected from the group consisting of F, Cl, Br, I, —NO₂, —CN, —OH, —SH, —NH₂, —N(C₁₋₅-alkyl)₂, —N(C₁₋₁₅-alkyl)(phenyl), —N(C₁₋₁₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the above-stated-C₁₋₅ alkyl residues may in each case be linear or branched and the above-stated phenyl residues may preferably be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl. Particularly preferred substituents may be selected mutually independently from the group consisting of F, Cl, Br, I, —NO₂, —CN, —OH, —SH, —NH₂, —N(CH₃)₂, —N(C₂H₅)₂ and —N(CH₃)(C₂H₅).

Examples which may be mentioned of suitable C₂₋₁₂ alkynyl residues are ethynyl, 1-propynyl, 2-propynyl, 1-butynyl, 2-butynyl, 3-butynyl, 1-pentynyl, 2-pentynyl, 3-pentynyl, 4-pentynyl and hexynyl.

Polysubstituted alkynyl residues should be taken to mean those alkynyl residues which are either polysubstituted on different C atoms, for example disubstituted on different C atoms as in the case of —CHCl—C≡CCl. Examples which may be mentioned of suitable substituted alkynyl residues are —C≡C—F, —C≡C—Cl and —C≡C—I.

The term “heteroalkyl” denotes an alkyl residue as described above, in which one or more C atoms have in each case been replaced by a heteroatom mutually independently selected from the group consisting of oxygen, sulfur and nitrogen (NH). Heteroalkyl residues preferably comprise 1, 2 or 3 heteroatom(s) mutually independently selected from the group consisting of oxygen, sulfur and nitrogen (NH) as chain link(s). Heteroalkyl residues may preferably be 2- to 12-membered, particularly preferably 2- to 6-membered.

Examples which may be mentioned of suitable heteroalkyl residues which may be unsubstituted or mono- or polysubstituted are —CH₂—O—CH₃, —CH₂—O—C₂H₅, —CH₂—O—CH(CH₃)₂, —CH₂—O—C(CH₃)₃, —CH₂—S—CH₃, —CH₂—S—C₂H₅, —CH₂—S—CH(CH₃)₂, —CH₂—S—C(CH₃)₃, —CH₂—NH—CH₃, —CH₂—NH—C₂H₅, —CH₂—NH—CH(CH₃)₂, —CH₂—NH—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—CH₂—S—CH₃, —CH₂—CH₂—S—C₂H₅, —CH₂—CH₂—S—CH(CH₃)₂, —CH₂—CH₂—S—C(CH₃)₃, —CH₂—CH₂—NH—CH₃, —CH₂—CH₂—NH—C₂H₅, —CH₂—CH₂—NH—CH(CH₃)₂, —CH₂—CH₂—NH—C(CH₃)₃, —CH₂—S—CH₂—O—CH₃, —CH₂—O—CH₂—O—C₂H₅, —CH₂—O—CH₂—O—CH(CH₃)₂, —CH₂—S—CH₂—O—C(CH₃)₃, —CH₂—O—CH₂—S—CH₃, —CH₂—O—CH₂—S—C₂H₅, —CH₂—O—CH₂—S—CH(CH₃)₂, —CH₂—NH—CH₂—S—C(CH₃)₃, —CH₂—O—CH₂—NH—CH₃, —CH₂—O—CH₂—NH—C₂H₅, —CH₂—O—CH₂—NH—CH(CH₃)₂, —CH₂—S—CH₂—NH—C(CH₃)₃ and —CH₂—CH₂—C(H)(CH₃)—(CH₂)₃—CH₃.

Examples which may be mentioned of suitable substituted heteroalkyl residues are —(CH₂)—O—(CF₃), —(CH₂)—O—(CHF₂), —(CH₂)—O—(CH₂F), —(CH₂)—S—(CF₃), —(CH₂)—S—(CHF₂), —(CH₂)—S—(CH₂F), —(CH₂)—(CH₂)—O—(CF₃), —(CF₂)—O—(CF₃), —(CH₂)—(CH₂)—S—(CF₃) and —(CH₂)—(CH₂)—(CH₂)—O—(CF₃).

The term “heteroalkenyl” denotes an alkenyl residue as described above in which one or more C atoms have in each case been replaced by a heteroatom mutually independently selected from the group consisting of oxygen, sulfur and nitrogen (NH). Heteroalkenyl groups may preferably comprise 1, 2 or 3 heteroatom(s) mutually independently selected from the group consisting of oxygen, sulfur and nitrogen (NH) as chain link(s). Heteroalkenyl groups may preferably be 2- to 12-membered, particularly preferably 2- to 6-membered.

Examples which may be mentioned of suitable heteroalkenyl residues are —CH₂—O—CH═CH₂, —CH═CH—O—CH═CH—CH₃, —CH₂—CH₂—O—CH═CH₂, —CH₂—S—CH═CH₂, —CH═CH—S—CH═CH—CH₃, —CH₂—CH₂—S—CH═CH₂, —CH₂—NH—CH═CH₂, —CH═CH—NH—CH═CH—CH₃ and —CH₂—CH₂—NH—CH═CH₂.

Examples which may be mentioned of suitable substituted heteroalkenyl residues are —CH₂—O—CH═CH—(CH₂)—OH, —CH₂—S—CH═CH—(CH₂)—NH₂ and —CH₂—NH—CH═CH—CN.

The term “heteroalkynyl” denotes an alkynyl residue as described above in which one or more C atoms have in each case been replaced by a heteroatom mutually independently selected from the group consisting of oxygen, sulfur and nitrogen (NH). Heteroalkynyl residues may preferably comprise 1, 2 or 3 heteroatom(s) mutually independently selected from the group consisting of oxygen, sulfur and nitrogen (NH) as chain link(s). Heteroalkynyl residues may preferably be 2- to 12-membered, particularly preferably 2- to 6-membered.

Examples which may be mentioned of suitable heteroalkynyl residues are —CH₂—O—C≡CH, —CH₂—CH₂—O—C≡CH, —CH₂—O—C≡C—CH₃, —CH₂—CH₂—O—C≡C—CH₃, —CH₂—S—C≡CH, —CH₂—CH₂—S—C≡CH, —CH₂—S—C≡C—CH₃, —CH₂—CH₂—S—C≡C—CH₃.

Examples which may be mentioned of suitable substituted heteroalkynyl residues are —CH₂—O—C═C—Cl, —CH₂—CH₂—O—C═C—I, —CHF—O—C═C—CH₃, —CHF—CH₂—O—C═C—CH₃, —CH₂—S—C═C—Cl, —CH₂—CH₂—S—C═C—Cl, —CHF—S—C═C—CH₃, —CHF—CH₂—S—C═C—CH₃.

For the purposes of the present invention, the term “cycloalkyl” means a cyclic saturated hydrocarbon residue, with preferably 3, 4, 5, 6, 7, 8 or 9 C atoms, particularly preferably with 3, 4, 5, 6 or 7 C atoms, very particularly preferably with 5 or 6 C atoms, wherein the residue may be unsubstituted or monosubstituted or identically or differently polysubstituted.

Examples which may be mentioned of suitable C₃₋₉ cycloalkyl residues which may be unsubstituted or mono- or polysubstituted are cyclopropyl, cyclobutyl, cyclopentyl, cyclohexyl, cycloheptyl, cyclooctyl and cyclononyl. Examples which may be mentioned of suitable C₃₋₇ cycloalkyl residues are cyclopropyl, cyclobutyl, cyclopentyl, cyclohexyl and cycloheptyl.

For the purposes of the present invention, the term “cycloalkenyl” means a cyclic unsaturated hydrocarbon residue with preferably 3, 4, 5, 6, 7, 8 or 9 C atoms, particularly preferably with 3, 4, 5, 6 or 7 C atoms, very particularly preferably with 5 or 6 C atoms, which comprises at least one double bond, preferably one double bond, and may be unsubstituted or monosubstituted or identically or differently polysubstituted.

Examples which may be mentioned of suitable C₃₋₉ cycloalkenyl residues which may be unsubstituted or mono- or polysubstituted are cyclobutenyl, cyclopentenyl, cyclohexenyl, cycloheptenyl, cyclononenyl and cyclooctenyl. Examples which may be mentioned of suitable C₅₋₆ cycloalkenyl residues are cyclopentenyl and cyclohexenyl.

For the purposes of the present invention, the term “heterocycloalkyl” means a cyclic saturated hydrocarbon residue with preferably 3, 4, 5, 6, 7, 8 or 9 C atoms, particularly preferably with 3, 4, 5, 6 or 7 C atoms, very particularly preferably with 5 or 6 C atoms, in which one or more C atoms have in each case been replaced by a heteroatom mutually independently selected from the group consisting of oxygen, sulfur and nitrogen (NH). Heterocycloalkyl residues may preferably comprise 1, 2 or 3 heteroatom(s) mutually independently selected from the group consisting of oxygen, sulfur and nitrogen (NH) as ring member(s). A heterocycloalkyl residue may be unsubstituted or monosubstituted or identically or differently polysubstituted. Heterocycloalkyl residues may preferably be 3- to 9-membered, particularly preferably 3- to 7-membered, very particularly preferably 5- to 7-membered.

Examples which may be mentioned of suitable 3- to 9-membered heterocycloalkyl residues which may be unsubstituted or mono- or polysubstituted are imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, azepanyl, azocanyl, diazepanyl, dithiolanyl, (1,3)-dioxolan-2-yl, isoxazolidinyl, isothioazolidinyl, pyrazolidinyl, oxazolidinyl, (1,2,4)-oxadiazolidinyl, (1,2,4)-thiadiazolidinyl, (1,2,4)-triazolidin-3-yl, (1,3,4)-thiadiazolidin-2-yl, (1,3,4)-triazolidin-1-yl, (1,3,4)-triazolidin-2-yl, tetrahydropyridazinyl, tetrahydropyrimidinyl, tetrahydropyrazinyl, (1,3,5)-tetrahydrotriazinyl, (1,2,4)-tetrahydrotriazin-1-yl, (1,3)-dithian-2-yl and (1,3)-thiazolidinyl. Examples which may be mentioned of suitable 5- to 7-membered heterocycloalkyl residues are imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, azepanyl, diazepanyl and (1,3)-dioxolan-2-yl.

For the purposes of the present invention, the term “heterocycloalkenyl” means a cyclic unsaturated hydrocarbon residue with preferably 4, 5, 6, 7, 8 or 9 C atoms, particularly preferably with 4, 5, 6 or 7 C atoms, very particularly preferably with 5 or 6 C atoms, which comprises at least one double bond, preferably one double bond, and in which one or more C atoms have in each case been replaced by a heteroatom mutually independently selected from the group consisting of oxygen, sulfur and nitrogen (NH). Heterocycloalkenyl residues may preferably comprise 1, 2 or 3 heteroatom(s) mutually independently selected from the group consisting of oxygen, sulfur and nitrogen (NH) as ring member(s). A heterocycloalkenyl residue may be unsubstituted or monosubstituted or identically or differently polysubstituted. Heterocycloalkenyl residues may preferably be 4- to 9-membered, particularly preferably 4- to 7-membered, very particularly preferably 5- to 7-membered.

Examples which may be mentioned of suitable heterocycloalkenyl residues or of suitable 5- to 7-membered heterocycloalkenyl residues which may be unsubstituted or mono- or polysubstituted are (2,3)-dihydrofuranyl, (2,5)-dihydrofuranyl, (2,3)-dihydrothienyl, (2,5)-dihydrothienyl, (2,3)-dihydropyrrolyl, (2,5)-dihydropyrrolyl, (2,3)-dihydroisoxazolyl, (4,5)-dihydroisoxazolyl, (2,5)-dihydroisothiazolyl, (2,3)-dihydropyrazolyl, (4,5)-dihydropyrazolyl, (2,5)-dihydropyrazolyl, (2,3)-dihydrooxazolyl, (4,5)-dihydrooxazolyl, (2,5)-dihydrooxazolyl, (2,3)-dihydrothiazolyl, (4,5)-dihydrothiazolyl, (2,5)-dihydrothiazolyl, (2,3)-dihydroimidazolyl, (4,5)-dihydroimidazolyl, (2,5)-dihydroimidazolyl, (3,4,5,6)-tetrahydropyridin-2-yl, (1,2,5,6)-tetrahydropyridin-1-yl, (1,2)-dihydropyridin-1-yl, (1,4)-dihydropyridin-1-yl, dihydropyranyl and (1,2,3,4)-tetrahydropyridin-1-yl.

The cycloalkyl residue, heterocycloalkyl residue, cycloalkenyl residue or heterocyclalkenyl residue may for the purposes of the present invention be fused (anellated) with an unsubstituted or at least monosubstituted mono- or bicyclic ring system. For the purposes of the present invention, a mono- or bicyclic ring system should be understood to mean mono- or bicyclic hydrocarbon residues which may be saturated, unsaturated or aromatic and optionally comprise one or more heteroatoms as ring members. Preferably, the rings of the above-stated mono- or bicyclic ring systems are in each case 4-, 5- or 6-membered and may in each case preferably optionally comprise 0, 1, 2, 3, 4 or 5 heteroatom(s), particularly preferably optionally 0, 1 or 2 heteroatom(s) as ring member(s), which are mutually independently selected from the group consisting of oxygen, nitrogen and sulfur. If a bicyclic ring system is present, the different rings may, in each case mutually independently, exhibit a different degree of saturation, i.e. be saturated, unsaturated or aromatic.

If one or more of the substituents comprise a monocyclic or bicyclic ring system, which is mono- or polysubstituted, this may preferably be substituted with optionally 1, 2, 3, 4 or 5, particularly preferably with optionally 1, 2 or 3, substituents, which may be mutually independently selected from the group consisting of F, Cl, Br, I, —CN, —NO₂, —OH, —SH, —NH₂, oxo (═O), thioxo (═S), —C(═O)—OH, C₁₋₅ alkyl, —C₂₋₅ alkenyl, —C₂₋₅ alkynyl, —C≡C—Si(CH₃)₃, —C═C—Si(C₂H₅)₃, —(CH₂)—O—C₁₋₅-alkyl, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅alkyl)(C₁₋₅-alkyl), —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, pyrazolyl, phenyl, furyl (furanyl), thiadiazolyl, thiophenyl (thienyl) and benzyl, wherein the above-stated C₁₋₅ alkyl residues may in each case be linear or branched and the cyclic substituents or the cyclic residues of these substituents may themselves in each case be substituted with optionally 1, 2, 3, 4 or 5, preferably with optionally 1, 2, 3 or 4, substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C═C—Si(CH₃)₃, —C═C—Si(C₂H₅)₃, —C(═O)—O—C₁₋₅-alkyl and —C(═O)—CF₃.

Particularly preferably, the substituents may, in each case mutually independently, be selected from the group consisting of F, Cl, Br, I, —CN, —NO₂, —OH, —SH, methyl, ethyl, n-propyl, isopropyl, n-butyl, isobutyl, 2-butyl, tert.-butyl, n-pentyl, neopentyl, ethenyl, allyl, ethynyl, propynyl, —C≡C—Si(CH₃)₃, —C≡C—Si(C₂H₅)₃, —C≡C—Si(CH₃)₃, —C≡C—Si(C₂H₅)₃, —CH₂—O—CH₃, —CH₂—O—C₂H₅, —OH, —SH, —NH₂, Oxo (═O), —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —NH—S(═O)₂—CH₃, —C(═O)—O—CH₃, —C(═O)—O—C₂H₅, —C(═O)—O—C(CH₃)₃, —C(═O)—H, —C(═O)—CH₃, —C(═O)—C₂H₅, —NH—C(═O)—CH₃, —NH—C(═O)—C₂H₅, —O—C(═O)-phenyl, —C(═O)—NH₂, —C(═O)—NH—CH₃, —C(═O)—N(CH₃)₂, phenyl, furyl (furanyl), thiadiazolyl, thiophenyl (thienyl) and benzyl, wherein the cyclic substituents or the cyclic residues of these substituents may themselves be substituted with optionally 1, 2, 3, 4 or 5, preferably with optionally 1, 2, 3 or 4, substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl, tert.-butyl, ethenyl, allyl, ethynyl, propynyl, —C≡C—Si(CH₃)₃, —C≡C—Si(C₂H₅)₃, —C(═O)—O—C₁₋₅-alkyl and —C(═O)—CF₃.

Examples which may be mentioned of suitable cycloalkyl residues, heterocycloalkyl residues, cycloalkenyl residues or heterocyclalkenyl residues which may be unsubstituted or mono- or polysubstituted, and are fused with a mono- or bicyclic ring system, are (1,2,3,4)-tetrahydroquinolinyl, (1,2,3,4)-tetrahydroisoquinolinyl, (2,3)-dihydro-1H-isoindolyl, (1,2,3,4)-tetrahydronaphthyl, (2,3)-dihydrobenzo[1.4]dioxinyl, benzo[1.3]dioxolyl, (3,4)-dihydro-2H-benzo[1.4]oxazinyl and octahydro-pyrrolo[3,4-c]pyrrolyl.

If one or more of the substituents denote a cycloalkyl residue, heterocycloalkyl residue, cycloalkenyl residue or heterocycloalkenyl residue or comprise such a residue, which is mono- or polysubstituted, this may preferably be substituted with optionally 1, 2, 3, 4 or 5, particularly preferably with optionally 1, 2 or 3, substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, wherein the above-stated-C₁₋₅ alkyl residues may in each case be linear or branched and the phenyl residues may in each case be unsubstituted or substituted with 1, 2, 3, 4 or 5, preferably with 1, 2, 3 or 4, substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C≡C—Si(CH₃)₃, —C═C—Si(C₂H₅)₃, —C(═O)—O—C₁₋₅-alkyl and —C(═O)—CF₃.

Particularly preferably, the substituents may, in each case mutually independently, be selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, —C≡C—Si(CH₃)₃, —C═C—Si(C₂H₅)₃, —OH, oxo, thioxo, —O—CH₃, —O—C₂H₅, —O—C₃H₇, —(CH₂)—O—CH₃, —(CH₂)—O—C₂H₅, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —S(═O)—CH₃, —S(═O)₂—CH₃, —S(═O)—C₂H₅, —S(═O)₂—C₂H₅, —NH—S(═O)₂—CH₃, —C(═O)—OH, —C(═O)—H; —C(═O)—CH₃, —C(═O)—C₂H₅, —C(═O)—N(CH₃)₂, —C(═O)—NH—CH₃, —C(═O)—NH₂, —NH—C(═O)—CH₃, —NH—C(═O)—C₂H₅, —C(═O)—O—CH₃, —C(═O)—O—C₂H₅, —C(═O)—O—C(CH₃)₃ and phenyl, wherein the phenyl residue may be substituted with 1, 2, 3, 4 or 5, preferably 1, 2 or 3, substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl, tert.-butyl, ethenyl, allyl, ethynyl, propynyl, —C≡C—Si(CH₃)₃, —C═C—Si(C₂H₅)₃, —C(═O)—O—C₁₋₅-alkyl and —C(═O)—CF₃.

For the purposes of the present invention, the term “aryl” means a mono- or polycyclic, preferably a mono- or bicyclic, aromatic hydrocarbon residue with preferably 6, 10 or 14 C atoms. An aryl residue may be unsubstituted or monosubstituted or identically or differently polysubstituted. Examples of suitable aryl residues which may be mentioned are phenyl, 1-naphthyl, 2-naphthyl and anthracenyl. A aryl residue is particularly preferably a phenyl residue.

For the purposes of the present invention, the term “heteroaryl” means a monocyclic or polycyclic, preferably a mono-, bi- or tricyclic aromatic hydrocarbon residue with preferably 5, 6, 7, 8, 9, 10, 11, 12, 13 or 14 C atoms, particularly preferably with 5, 6, 9, 10, 13 or 14 C atoms, very particularly preferably with 5 or 6 C atoms, in which one or more C atoms have in each case been replaced by a heteroatom mutually independently selected from the group consisting of oxygen, sulfur and nitrogen (NH). Heteroaryl residues may preferably comprise 1, 2, 3, 4 or 5, particularly preferably 1, 2 or 3, heteroatom(s) mutually independently selected from the group consisting of oxygen, sulfur and nitrogen (NH) as ring member(s) A heteroaryl residue may be unsubstituted or monosubstituted or identically or differently polysubstituted.

Examples which may be mentioned of suitable heteroaryl residues are indolizinyl, benzimidazolyl, tetrazolyl, triazinyl, isoxazolyl, phthalazinyl, carbazolyl, carbolinyl, diaza-naphthyl, thienyl, furyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, isoxazolyl, pyridazinyl, pyrimidinyl, indazolyl, quinoxalinyl, quinazolinyl, quinolinyl, naphthridinyl and isoquinolinyl.

For the purposes of the present invention aryl or heteroaryl residues may be fused (anellated) with a mono- or bicyclic ring system.

Examples which may be mentioned of aryl residues which are fused with a mono- or bicyclic ring system are (2,3)-dihydrobenzo[b]thiophenyl, (2,3)-dihydro-1H-indenyl, indolinyl, (2,3)-dihydrobenzofuranyl, (2,3)-dihydrobenzo[d]oxazolyl, benzo[d][1,3]dioxolanyl, benzo[d][1,3]oxathiolyl, isoindolinyl, (1,3)-diyhydroisobenzofuranyl, (1,3)-dihydrobenzo[c]thiophenyl, (1,2,3,4)-tetrahydronaphthyl, (1,2,3,4)-tetrahydroquinolinyl, chromanyl, thiochromanyl, (1,2,3,4)-tetrahydroisoquinolinyl, (1,2,3,4)-tetrahydroquinoxalinyl, (3,4)-dihydro-2H-benzo[1,4]oxazinyl, (3,4)-dihydro-2H-benzo[b][1,4]thiazinyl, (2,3)-dihydrobenzo[b][1,4]dioxinyl, (2,3)-dihydrobenzo[b][1,4]oxathiinyl, (6,7,8,9)-tetrahydro-5H-benzo[7]annulenyl, (2,3,4,5)-tetrahydro-1H-benzo[b]azepinyl and (2,3,4,5)-tetrahydro-1H-benzo[c]azepinyl.

Unless otherwise indicated, if one or more of the substituents denote an aryl or heteroaryl residue or comprise an aryl or heteroaryl residue, which is mono- or polysubstituted, these aryl or heteroaryl residues may preferably be substituted with optionally 1, 2, 3, 4 or 5, particularly preferably optionally 1, 2 or 3, substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)—C₁₋₅-alkyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, pyrazolyl, phenyl, furyl (furanyl), thiazolyl, thiadiazolyl, thiophenyl (thienyl)benzyl and phenethyl, wherein the above-stated C₁₋₅ alkyl residues may in each case be linear or branched and the cyclic substituents or the cyclic residues of these substituents themselves may be substituted with optionally 1, 2, 3, 4 or 5, preferably with optionally 1, 2, 3 or 4, substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F.

Particularly preferably, the substituents may be in each case mutually independently selected from the group consisting of F, Cl, Br, I, —CN, —NO₂, —OH, —SH, methyl, ethyl, n-propyl, isopropyl, n-butyl, isobutyl, 2-butyl, tert.-butyl, n-pentyl, neopentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —NH—S(═O)₂—CH₃, —C(═O)—O—CH₃, —C(═O)—O—C₂H₅, —C(═O)—O—C(CH₃)₃, —C(═O)—H, —C(═O)—CH₃, —C(═O)—C₂H₅, —NH—C(═O)—CH₃, —NH—C(═O)—C₂H₅, —O—C(═O)-phenyl, —C(═O)—NH₂, —C(═O)—NH—CH₃, —C(═O)—N(CH₃)₂, —C(═O)—O—CH(CH₃)₂, —C(═O)—O—(CH₂)₃—CH₃, —C(═O)—O-phenyl, —O—C(═O)—CH₃, —O—C(═O)—C₂H₅, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, phenyl, furyl (furanyl), thiadiazolyl, thiophenyl (thienyl) and benzyl, wherein the cyclic substituents or the cyclic residues of these substituents themselves may in each case be substituted with optionally 1, 2, 3, 4, or 5, preferably with optionally 1, 2, 3 or 4, substituents mutually independently selected from the group consisting of F, Cl, Br, I, —CN, —NO₂, —OH, —SH, —NH₂, —C(═O)—OH, methyl, ethyl, n-propyl, isopropyl, n-butyl, isobutyl, 2-butyl, tert.-butyl, n-pentyl, neopentyl, ethenyl, allyl, ethynyl, propynyl, —C≡C—Si(CH₃)₃, —C≡C—Si(C₂H₅)₃, —CH₂—O—CH₃, —CH₂—O—C₂H₅, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂ and —S—CH₂F.

Very particularly preferably, a substituted aryl residue may be selected from the group consisting of 2-methylphenyl, 3-methylphenyl, 4-methylphenyl, 2-fluorophenyl, 3-fluorophenyl, 4-fluorophenyl, 2-cyanophenyl, 3-cyanophenyl, 4-cyanophenyl, 2-hydroxyphenyl, 3-hydroxyphenyl, 4-hydroxyphenyl, 2-aminophenyl, 3-aminophenyl, 4-aminophenyl, 2-dimethylaminophenyl, 3-dimethylaminophenyl, 4-dimethylaminophenyl, 2-methylaminophenyl, 3-methylaminophenyl, 4-methylaminophenyl, 2-acetylphenyl, 3-acetylphenyl, 4-acetylphenyl, 2-methylsulfinylphenyl, 3-methylsulfinylphenyl, 4-methylsulfinylphenyl, 2-methylsulfonylphenyl, 3-methylsulfonylphenyl, 4-methylsulfonylphenyl, 2-methoxyphenyl, 3-methoxyphenyl, 4-methoxyphenyl, 2-chlorophenyl, 3-chlorophenyl, 4-chlorophenyl, 2-ethoxyphenyl, 3-ethoxyphenyl, 4-ethoxyphenyl, 2-trifluoromethylphenyl, 3-trifluoromethylphenyl, 4-trifluoromethylphenyl, 2-difluoromethylphenyl, 3-difluoromethylphenyl, 4-difluoromethylphenyl, 2-fluoromethylphenyl, 3-fluoromethylphenyl, 4-fluoromethylphenyl, 2-nitrophenyl, 3-nitrophenyl, 4-nitrophenyl, 2-ethylphenyl, 3-ethylphenyl, 4-ethylphenyl, 2-propylphenyl, 3-propylphenyl, 4-propylphenyl, 2-isopropylphenyl, 3-isopropylphenyl, 4-isopropylphenyl, 2-tert.-butylphenyl, 3-tert.-butylphenyl, 4-tert.-butylphenyl, 2-carboxyphenyl, 3-carboxyphenyl, 4-carboxyphenyl, 2-ethenylphenyl, 3-ethenylphenyl, 4-ethenylphenyl, 2-ethynylphenyl, 3-ethynylphenyl, 4-ethynylphenyl, 2-allylphenyl, 3-allylphenyl, 4-allylphenyl, 2-trimethylsilanylethynylphenyl, 3-trimethylsilanylethynylphenyl, 4-trimethylsilanylethynylphenyl, 2-formylphenyl, 3-formylphenyl, 4-formylphenyl, 2-acetaminophenyl, 3-acetaminophenyl, 4-acetaminophenyl, 2-dimethylaminocarbonylphenyl, 3-dimethylaminocarbonylphenyl, 4-dimethylaminocarbonylphenyl, 2-methoxymethylphenyl, 3-methoxymethylphenyl, 4-methoxymethylphenyl, 2-ethoxymethylphenyl, 3-ethoxymethylphenyl, 4-ethoxymethylphenyl, 2-aminocarbonylphenyl, 3-aminocarbonylphenyl, 4-aminocarbonylphenyl, 2-methylaminocarbonylphenyl, 3-methylaminocarbonylphenyl, 4-methylaminocarbonylphenyl, 2-carboxymethyl ester phenyl, 3-carboxymethyl ester phenyl, 4-carboxymethyl ester phenyl, 2-carboxyethyl ester phenyl, 3-carboxyethyl ester phenyl, 4-carboxyethyl ester phenyl, 2-carboxy tert.-butyl ester phenyl, 3-carboxy tert.-butyl ester phenyl, 4-carboxy tert.-butyl ester phenyl, 2-methylmercaptophenyl, 3-methylmercaptophenyl, 4-methylmercaptophenyl, 2-ethyl mercaptophenyl, 3-ethyl mercaptophenyl, 4-ethyl mercaptophenyl, 2-biphenyl, 3-biphenyl, 4-biphenyl, 2-bromophenyl, 3-bromophenyl, 4-bromophenyl, 2-iodophenyl, 3-iodophenyl, 4-iodophenyl, 2-trifluoromethoxyphenyl, 3-trifluoromethoxyphenyl, 4-trifluoromethoxyphenyl, 2-fluoro-3-trifluoromethylphenyl, 2-fluoro-4-methylphenyl, (2,3)-difluorophenyl, (2,3)-dimethylphenyl, (2,3)-dichlorophenyl, 3-fluoro-2-trifluoromethylphenyl, (2,4)-dichlorophenyl, (2,4)-difluorophenyl, 4-fluoro-2-trifluoromethylphenyl, (2,4)-dimethoxyphenyl, 2-chloro-4-fluorophenyl, 2-chloro-4-nitrophenyl, 2-chloro-4-methylphenyl, 2-chloro-5-trifluoromethylphenyl, 2-chloro-5-methoxyphenyl, 2-bromo-5-trifluoromethylphenyl, 2-bromo-5-methoxyphenyl, (2,4)-dibromophenyl, (2,4)-dimethylphenyl, 2-fluoro-4-trifluoromethylphenyl, (2,5)-difluorophenyl, 2-fluoro-5-trifluoromethylphenyl, 5-fluoro-2-trifluoromethylphenyl, 5-chloro-2-trifluoromethylphenyl, 5-bromo-2-trifluoromethylphenyl, (2,5)-dimethoxyphenyl, (2,5)-bis-trifluoromethylphenyl, (2,5)-dichlorophenyl, (2,5)-dibromophenyl, 2-methoxy-5-nitrophenyl, 2-fluoro-6-trifluoromethylphenyl, (2,6)-dimethoxyphenyl, (2,6)-dimethylphenyl, (2,6)-dichlorophenyl, 2-chloro-6-fluorophenyl, 2-bromo-6-chlorophenyl, 2-bromo-6-fluorophenyl, (2,6)-difluorophenyl, (2,6)-difluoro-3-methylphenyl, (2,6)-dibromophenyl, (2,6)-dichlorophenyl, 3-chloro-2-fluorophenyl, 3-chloro-5-methylphenyl, (3,4)-dichlorophenyl, (3,4)-dimethylphenyl, 3-methyl-4-methoxyphenyl, 4-chloro-3-nitrophenyl, (3,4)-dimethoxyphenyl, 4-fluoro-3-trifluoromethylphenyl, 3-fluoro-4-trifluoromethylphenyl, (3,4)-difluorophenyl, 3-cyano-4-fluorophenyl, 3-cyano-4-methylphenyl, 3-cyano-4-methoxyphenyl, 3-bromo-4-fluorophenyl, 3-bromo-4-methylphenyl, 3-bromo-4-methoxyphenyl, 4-chloro-2-fluorophenyl, 4-chloro-3-trifluoromethyl, 4-bromo-3-methylphenyl, 4-bromo-5-methylphenyl, 3-chloro-4-fluorophenyl, 4-fluoro-3-nitrophenyl, 4-bromo-3-nitrophenyl, (3,4)-dibromophenyl, 4-chloro-3-methylphenyl, 4-bromo-3-methylphenyl, 4-fluoro-3-methylphenyl, 3-fluoro-4-methylphenyl, 3-fluoro-5-methylphenyl, 2-fluoro-3-methylphenyl, 4-methyl-3-nitrophenyl, (3,5)-dimethoxyphenyl, (3,5)-dimethylphenyl, (3,5)-bis-trifluoromethylphenyl, (3,5)-difluorophenyl, (3,5)-dinitrophenyl, (3,5)-dichlorophenyl, 3-fluoro-5-trifluoromethylphenyl, 5-fluoro-3-trifluoromethylphenyl, (3,5)-dibromophenyl, 5-chloro-4-fluorophenyl, 5-chloro-4-fluorophenyl, 5-bromo-4-methylphenyl, (2,3,4)-trifluorophenyl, (2,3,4)-trichlorophenyl, (2,3,6)-trifluorophenyl, 5-chloro-2-methoxyphenyl, (2,3)-difluoro-4-methyl, (2,4,5)-trifluorophenyl, (2,4,5)-trichlorophenyl, (2,4)-dichloro-5-fluorophenyl, (2,4,6)-trichlorophenyl, (2,4,6)-trimethylphenyl, (2,4,6)-trifluorophenyl, (2,4,6)-trimethoxyphenyl, (3,4,5)-trimethoxyphenyl, (2,3,4,5)-tetrafluorophenyl, 4-methoxy-(2,3,6)-trimethylphenyl, 4-methoxy-(2,3,6)-trimethylphenyl, 4-chloro-2,5-dimethylphenyl, 2-chloro-6-fluoro-3-methylphenyl, 6-chloro-2-fluoro-3-methyl, (2,4,6)-trimethylphenyl and (2,3,4,5,6)-pentafluorophenyl.

Very particularly preferably, a substituted heteroaryl residue may be selected from the group consisting of 3-methylpyrid-2-yl, 4-methylpyrid-2-yl, 5-methylpyrid-2-yl, 6-methylpyrid-2-yl, 2-methylpyrid-3-yl, 4-methylpyrid-3-yl, 5-methylpyrid-3-yl, 6-methylpyrid-3-yl, 2-methylpyrid-4-yl, 3-methylpyrid-4-yl, 3-fluoropyrid-2-yl, 4-fluoropyrid-2-yl, 5-fluoropyrid-2-yl, 6-fluoropyrid-2-yl, 3-chloropyrid-2-yl, 4-chloropyrid-2-yl, 5-chloropyrid-2-yl, 6-chloropyrid-2-yl, 3-trifluoromethylpyrid-2-yl, 4-trifluoromethylpyrid-2-yl, 5-trifluoromethylpyrid-2-yl, 6-trifluoromethylpyrid-2-yl, 3-methoxypyrid-2-yl, 4-methoxypyrid-2-yl, 5-methoxypyrid-2-yl, 6-methoxypyrid-2-yl, 4-methylthiazol-2-yl, 5-methylthiazol-2-yl, 4-trifluoromethylthiazol-2-yl, 5-trifluoromethylthiazol-2-yl, 4-chlorothiazol-2-yl, 5-chlorothiazol-2-yl, 4-bromothiazol-2-yl, 5-bromothiazol-2-yl, 4-fluorothiazol-2-yl, 5-fluorothiazol-2-yl, 4-cyanothiazol-2-yl, 5-cyanothiazol-2-yl, 4-methoxythiazol-2-yl, 5-methoxythiazol-2-yl, 4-methyloxazol-2-yl, 5-methyloxazol-2-yl, 4-trifluoromethyloxazol-2-yl, 5-trifluoromethyloxazol-2-yl, 4-chlorooxazol-2-yl, 5-chlorooxazol-2-yl, 4-bromooxazol-2-yl, 5-bromooxazol-2-yl, 4-fluorooxazol-2-yl, 5-fluorooxazol-2-yl, 4-cyanooxazol-2-yl, 5-cyanooxazol-2-yl, 4-methoxyoxazol-2-yl, 5-methoxyoxazol-2-yl, 2-methyl-(1,2,4)-thiadiazol-5-yl, 2-trifluoromethyl-(1,2,4)-thiadiazol-5-yl, 2-chloro-(1,2,4)-thiadiazol-5-yl, 2-fluoro-(1,2,4)-thiadiazol-5-yl, 2-methoxy-(1,2,4)-thiadiazol-5-yl, 2-cyano-(1,2,4)-thiadiazol-5-yl, 2-methyl-(1,2,4)-oxadiazol-5-yl, 2-trifluoromethyl-(1,2,4)-oxadiazol-5-yl, 2-chloro-(1,2,4)-oxadiazol-5-yl, 2-fluoro-(1,2,4)-oxadiazol-5-yl, 2-methoxy-(1,2,4)-oxadiazol-5-yl and 2-cyano-(1,2,4)-oxadiazol-5-yl.

For the purposes of the present invention, the term “alkylene” covers acyclic saturated hydrocarbon chains, which combine an aryl, heteroaryl, cycloalkyl, heterocycloalkyl, cycloalkenyl or heterocycloalkenyl residue with the compounds of the general formula I or with another substituent. Alkylene chains may be branched or straight-chain and unsubstituted or at least monosubstituted with, as in the case of C₁₋₁₂ alkylene, 1 to 12 (i.e. 1, 2, 3, 4, 5, 6, 7, 8, 9, 10, 11 or 12) C atoms, with, as in the case of C₁₋₆ alkylene, 1 to 6 (i.e. 1, 2, 3, 4, 5 or 6) C atoms or with, as in the case of C₁₋₃ alkylene, 1 to 3 (i.e. 1, 2 or 3) C atoms. Examples which may be mentioned are C₁₋₆ alkylene groups such as —(CH₂)—, —(CH₂)₂—, —C(H)(CH₃)—, —(CH₂)₃—, —(CH₂)₄—, —(CH₂)₅—, —C(CH₃)₂—, —C(H)(CH₃)—, —C(H)(C(H)(CH₃)₂)— and C(C₂H₅)(H)—. Examples of suitable C₁₋₃ alkylene groups are —(CH₂)—, —(CH₂)₂— and —(CH₂)₃—.

For the purposes of the present invention, the term “alkenylene” covers acyclic unsaturated hydrocarbon chains, which combine an aryl, heteroaryl, cycloalkyl, heterocycloalkyl, cycloalkenyl or heterocycloalkenyl residue with the compounds of the general formula I or with another substituent. Alkenylene chains comprise at least one double bond, preferably 1, 2 or 3 double bonds, and may be branched or straight-chain and unsubstituted or at least monosubstituted with, as in the case of C₂₋₁₂ alkenylene, 2 to 12 (i.e. 2, 3, 4, 5, 6, 7, 8, 9, 10, 11 or 12) C atoms, with, as in the case of C₂₋₆ alkenylene, 2 to 6 (i.e. 2, 3, 4, 5 or 6) C atoms or with, as in the case of C₂₋₃ alkenylene, 2 to 3 (i.e. 2 or 3) C atoms. Examples which may be mentioned are C₂₋₃ alkenylene groups such as —CH═CH— and —CH₂—CH═CH.

For the purposes of the present invention, the term “alkynylene” covers acyclic unsaturated hydrocarbon chains, which combine an aryl, heteroaryl, cycloalkyl, heterocycloalkyl, cycloalkenyl or heterocycloalkenyl residue with the compounds of the general formula I or with another substituent. Alkynylene chains comprise at least one triple bond, preferably 1 or 2 triple bonds, and may be branched or straight-chain and unsubstituted or at least monosubstituted with, as in the case of C₂₋₁₂ alkynylene, 2 to 12 (i.e. 2, 3, 4, 5, 6, 7, 8, 9, 10, 11 or 12) C atoms, with, as in the case of C₂₋₆ alkynylene, 2 to 6 (i.e. 2, 3, 4, 5 or 6) C atoms or with, as in the case of C₂₋₃ alkynylene, 2 to 3 (i.e. 2 or 3) C atoms. Examples which may be mentioned are C₂₋₃ alkynylene groups such as —C≡C— and —CH₂—C═C—.

The term “heteroalkylene” denotes an alkylene chain as described above in which one or more C atoms have in each case been replaced by a heteroatom mutually independently selected from the group consisting of oxygen, sulfur and nitrogen (NH). Heteroalkylene groups may preferably comprise 1, 2 or 3 heteroatom(s), particularly preferably one heteroatom, selected from the group consisting of oxygen, sulfur and nitrogen (NH) as chain link(s). Heteroalkylene groups may preferably be 2- to 12-membered, particularly preferably 2- to 6-membered, very particularly preferably 2- or 3-membered.

Examples which may be mentioned are heteroalkylene groups such as —(CH₂)—O—, —(CH₂)₂—O—, —(CH₂)₃—O—, —(CH₂)₄—O—, —O—(CH₂)—, —O—(CH₂)₂—, —O—(CH₂)₃—, —O—(CH₂)₄—, —C(C₂H₅)(H)—O—, —O—C(C₂H₅)(H)—, —CH₂—O—CH₂—, —CH₂—S—CH₂—, —CH₂—NH—CH₂—, —CH₂—NH— and —CH₂—CH₂—NH—CH₂—CH₂

The term “heteroalkenylene” denotes an alkenylene chain as described above in which one or more C atoms have in each case been replaced by a heteroatom mutually independently selected from the group consisting of oxygen, sulfur and nitrogen (NH). Heteroalkenylene groups may preferably comprise 1, 2 or 3 heteroatom(s), particularly preferably 1 heteroatom, selected from the group consisting of oxygen, sulfur and nitrogen (NH) as chain link(s). Heteroalkenylene groups may preferably be 2- to 12-membered, particularly preferably 2- to 6-membered, very particularly preferably 2- or 3-membered. Examples which may be mentioned are heteroalkenylene groups such as —CH═CH—NH—, —CH═CH—O— and —CH═CH—S—.

If one or more of the substituents denote an alkylene, alkenylene, alkynylene, heteroalkylene or heteroalkenylene group or comprise such a group, which may be mono- or polysubstituted, this may preferably be substituted with optionally 1, 2, 3, 4 or 5, particularly preferably with optionally 1, 2 or 3, substituents mutually independently selected from the group consisting of phenyl, F, Cl, Br, I, —NO₂, —CN, —OH, —O-phenyl, —O—CH₂-phenyl, —SH, —S-phenyl, —S—CH₂-phenyl, —NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the above-stated-C₁₋₅ alkyl residues may in each case be linear or branched and the above-stated phenyl residues may be substituted with 1, 2, 3, 4 or 5, preferably with 1, 2, 3 or 4, substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F.

Particularly preferably, alkylene, alkenylene, alkynylene, heteroalkylene or heteroalkenylene groups may be substituted with 1, 2 or 3 substituents mutually independently selected from the group consisting of phenyl, F, Cl, Br, I, —NO₂, —CN, —OH, —O-phenyl, —SH, —S-phenyl, —NH₂, —N(CH₃)₂, —N(C₂H₅)₂ and —N(CH₃)(C₂H₅), wherein the phenyl residue may be substituted with 1, 2, 3, 4 or 5 substituents mutually independently selected from the group consisting of F, Cl, Br, I, —OH, —SH, —NO₂, —CN, —O—CH₃, —O—CF₃, and —O—C₂H₅.

Preferred propiolic acid amides are those of the above-stated general formula I, in which

R¹ denotes unsubstituted or substituted aryl or unsubstituted or substituted heteroaryl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
or R² and R³ or R⁴ and R⁵ or R⁶ and R⁷ or R⁸ and R⁹ or R¹⁰ and R¹¹ or R¹² and R¹³ in each case together denote an oxo group (═O) or a thioxo group (═S);
M and W, mutually independently, in each case denote N or C;
P denotes CR¹⁴, N, NR¹⁵, O or S;
Q denotes CR¹⁶, N, NR¹⁷, O or S;
T denotes CR¹⁸, N, NR¹⁹, O or S;
V denotes CR²⁰C, N, NR², O or S;
g denotes 0 or 1:
wherein M, W; P, Q, T, V and g together form a 5- or 6-membered aromatic or heteroaromatic ring;
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴—O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷—NH—C(═O)—NH—R³⁸; —NH—C(═S)—NH—R³⁹; —NH—S(═O)₂—R⁴⁰; —NR⁴¹—S(═O)₂—R⁴²; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
R¹⁵, R¹⁷, R¹⁹ and R²¹, mutually independently, in each case denote H; —C(═O)—OH; —C(═O)—H; —C(═O)—R²⁷; —C(═O)—O—R²⁸; —C(═O)—NH₂; —C(═O)—NH—R³³; —C(═O)—NR³⁴R³⁵; —S(═O)—R³⁶; —S(═O)₂—R³⁷; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
and R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴², mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
with the proviso that compounds are excepted in which the sum of a, b, c, d, e and f is equal to 3, W and M in each case denote C, P denotes CR¹⁴, Q denotes CR¹⁶, T denotes CR¹⁸ and V denotes CR²⁰;
with the proviso that compounds are excepted in which g is 0, W and M in each case denote C, P denotes NR¹⁵, Q denotes CR¹⁶ and T denotes N;
and with the proviso that compounds are excepted in which g is 0, W and M in each case denote C, P denotes N, Q denotes CR¹⁶ and T denotes NR¹⁹;
wherein
the above-stated alkyl residues are in each case branched or straight-chain and comprise 1, 2, 3, 4, 5, 6, 7, 8, 9, 10, 11 or 12 carbon atoms as chain links; the above-stated alkenyl residues are in each case branched or straight-chain and comprise 2, 3, 4, 5, 6, 7, 8, 9, 10, 11 or 12 carbon atoms as chain links; the above-stated alkynyl residues are in each case branched or straight-chain and comprise 2, 3, 4, 5, 6, 7, 8, 9, 10, 11 or 12 carbon atoms as chain links; the above-stated heteroalkyl residues, heteroalkenyl residues and heteroalkynyl residues are in each case 2-, 3-, 4-, 5-, 6-, 7-, 8-, 9-, 10-, 11- or 12-membered; the above-stated heteroalkyl residues, heteroalkenyl residues and heteroalkynyl residues in each case optionally comprise 1, 2 or 3 heteroatom(s) mutually independently selected from the group consisting of oxygen, nitrogen and sulfur (NH) as chain link(s);
the above-stated alkyl residues, alkenyl residues, alkynyl residues, heteroalkyl residues, heteroalkenyl residues and heteroalkynyl residues may be substituted in each case with optionally 1, 2, 3, 4 or 5 substituents mutually independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the phenyl residues may be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl; the above-stated cycloalkyl residues in each case comprise 3, 4, 5, 6, 7, 8 or 9 carbon atoms as ring members;
the above-stated cycloalkenyl residues in each case comprise 3, 4, 5, 6, 7, 8 or 9 carbon atoms as ring members;
the above-stated heterocycloalkyl residues are in each case 3-, 4-, 5-, 6-, 7-, 8- or 9-membered;
the above-stated heterocycloalkenyl residues are in each case 4-, 5-, 6-, 7-, 8- or 9-membered;
the above-stated heterocycloalkyl residues and heterocycloalkenyl residues in each case optionally comprise 1, 2 or 3 heteroatom(s) mutually independently selected from the group consisting of oxygen, nitrogen and sulfur (NH) as ring member(s);
the above-stated cycloalkyl residues, heterocycloalkyl residues, cycloalkenyl residues or heterocycloalkenyl residues may be substituted in each case with optionally 1, 2, 3, 4 or 5 substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C≡C—Si(CH₃)₃, —C═C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —C(═O)—H, —C(═O)—C₁₋₅-alkyl, —C(═O)—NH₂, —C(═O)—N(C₁₋₅-alkyl)₂, —C(═O)—N(H)(—C₁₋₅-alkyl) and phenyl, wherein the phenyl residues may in each case be unsubstituted or substituted with 1, 2, 3, 4 or 5 substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C≡C—Si(CH₃)₃, —C≡C—Si(C₂H₅)₃, —C(═O)—O—C₁₋₅-alkyl and —C(═O)—CF₃, wherein the above-stated phenyl residues may preferably be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl; the above-stated alkylene residues are in each case branched or straight-chain and comprise 1, 2, 3, 4, 5, 6, 7, 8, 9, 10, 11 or 12 carbon atoms as chain links; the above-stated alkenylene residues are in each case branched or straight-chain and comprise 2, 3, 4, 5, 6, 7, 8, 9, 10, 11 or 12 carbon atoms as chain links; the above-stated alkynylene residues are in each case branched or straight-chain and comprise 2, 3, 4, 5, 6, 7, 8, 9, 10, 11 or 12 carbon atoms as chain links; the above-stated heteroalkylene residues and heteroalkenylene residues are in each case 2-, 3-, 4-, 5-, 6-, 7-, 8-, 9-, 10-, 11- or 12-membered;
the above-stated heteroalkylene and heteroalkenylene groups may in each case optionally comprise 1, 2 or 3 heteroatom(s) mutually independently selected from the group consisting of oxygen, nitrogen and sulfur (NH) as chain link(s);
the above-stated alkylene, alkenylene, alkynylene, heteroalkylene or heteroalkenylene group may in each case be unsubstituted or substituted with optionally 1, 2, 3, 4 or 5 substituents mutually independently selected from the group consisting of phenyl, F, Cl, Br, I, —NO₂, —CN, —OH, —O-phenyl, —O—CH₂-phenyl, —SH, —S-phenyl, —S—CH₂-phenyl, NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the phenyl residues may be substituted with 1, 2, 3, 4 or 5 substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
the above-stated aryl residues are mono- or bicyclic and comprise 6, 10 or 14 carbon atoms;
the above-stated heteroaryl residues are mono-, di- or tricyclic and 5-, 6-, 7-, 8-, 9-, 10-, 11-, 12-, 13- or 14-membered;
the above-stated 5- to 14-membered heteroalkyl residues optionally comprise 1, 2, 3, 4 or 5 heteroatom(s) mutually independently selected from the group consisting of oxygen, nitrogen and sulfur (NH) as ring member(s);
and the above-stated aryl or heteroaryl residues may optionally in each case be substituted with 1, 2, 3, 4 or 5 substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₁₅-alkyl)₂, —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, wherein the cyclic substituents or the cyclic residues of these substituents may themselves be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Also preferred are propiolic acid amides of the above-stated general formula I, in which R¹ denotes a residue selected from the group consisting of phenyl, naphthyl, anthracenyl, furyl, thienyl, pyrazolyl, pyridazinyl, pyrazinyl, pyrimidinyl, pyridinyl, pyrrolyl, oxazolyl, isoxazolyl, thiazolyl, thiadiazolyl, oxadiazolyl, triazolyl, imidazolyl, indolyl, benzo[b]thiophenyl, benzo[d]thiazolyl, benzo[b]furanyl, quinolinyl, isoquinolinyl and quinazolinyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C═C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl; and in each case the other residues have the above-stated meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Preference is additionally given to propiolic acid amides of the above-stated general formula I, in which R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂ C₂₋₆ heteroalkyl, C₂₋₆ heteroalkenyl or C₂₋₆ heteroalkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂ and/or comprises 1 or 2 heteroatom(s) mutually independently selected from the group consisting of oxygen, sulfur and nitrogen (NH) as chain link(s); or C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or be unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;

or R² and R³ or R⁴ and R⁵ or R⁶ and R⁷ or R⁸ and R⁹ or R¹⁰ and R¹¹ or R¹² and R¹³ mutually independently, together in each case denote a residue selected from the group consisting of an oxo group (═O) or a thioxo group (═S);
and in each case the other residues have the above-stated meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Preference is likewise given to propiolic acid amides of the above-stated general formula I, in which W, M, P, Q, T and optionally V together denote a ring selected from the group consisting of

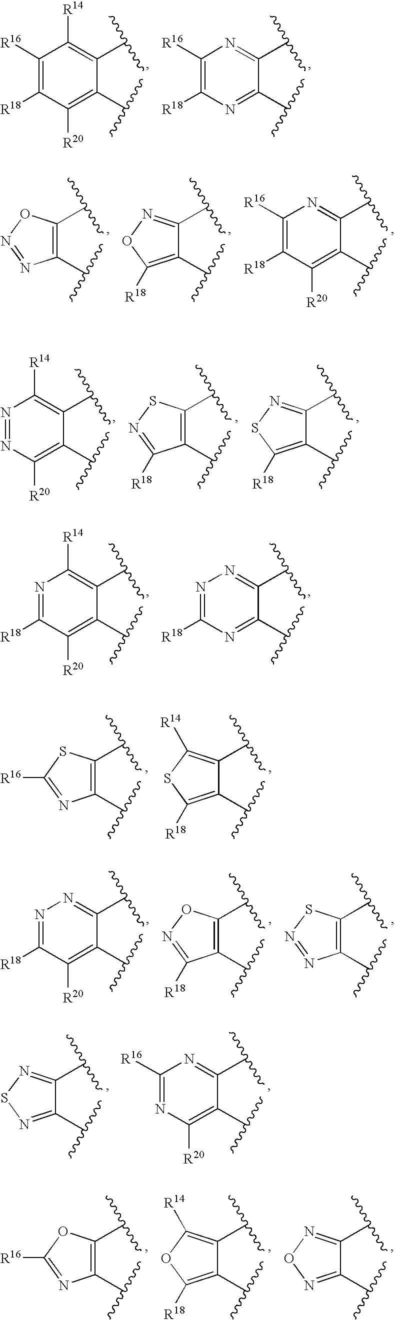

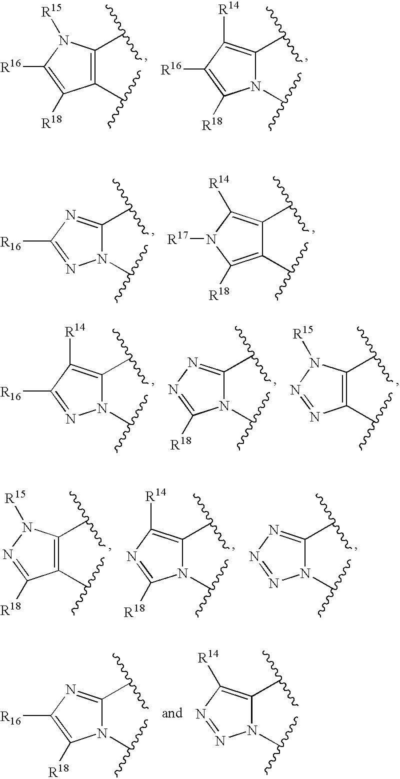

wherein the lines represent the bond in the parent structure;
and in each case the other residues have the above-stated meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Preference is additionally given to propiolic acid amides of the above-stated general formula I, in which R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²⁸—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂ C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;

and in each case the other residues have the above-stated meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Preference is likewise given to propiolic acid amides of the above-stated general formula I, in which R¹⁵, R¹⁷, R¹⁹ and R²¹, mutually independently, in each case denote H; —C(═O)—H; —C(═O)—R²⁷; —C(═O)—O—R²⁸; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene , C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;

and in each case the other residues have the above-stated meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Preference is additionally given to propiolic acid amides of the above-stated general formula I, in which R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴², mutually independently, in each case denote —C₁₋₆ alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, pyrrolyl, indolyl, furanyl, benzo[b]furanyl, thiophenyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃, and —C(═O)—O—C₂H₅;

and in each case the other residues have the above-stated meaning, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Particularly preferred propiolic acid amides are those of the above-stated general formula I, in which

R¹ denotes a residue selected from the group consisting of phenyl, naphthyl, anthracenyl, furyl, thienyl, pyrazolyl, pyridazinyl, pyrazinyl, pyrimidinyl, pyridinyl, pyrrolyl, oxazolyl, isoxazolyl, thiazolyl, thiadiazolyl, oxadiazolyl, triazolyl, imidazolyl, indolyl, benzo[b]thiophenyl, benzo[d]thiazolyl, benzo[b]furanyl, quinolinyl, isoquinolinyl and quinazolinyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C≡C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₂₋₆ heteroalkyl, C₂₋₆ heteroalkenyl or C₂₋₆ heteroalkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂ and/or comprises 1 or 2 heteroatom(s) mutually independently selected from the group consisting of oxygen, sulfur and nitrogen (NH) as chain link(s); or C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or be unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
or R² and R³ or R⁴ and R⁵ or R⁶ and R⁷ or R³ and R⁹ or R¹⁰ and R¹¹ or R¹² and R¹³ mutually independently, together in each case denote a residue selected from the group consisting of an oxo group (═O) or a thioxo group (═S);
g denotes 0 or 1;
W, M, P, Q, T and optionally V together denote a ring selected from the group consisting of

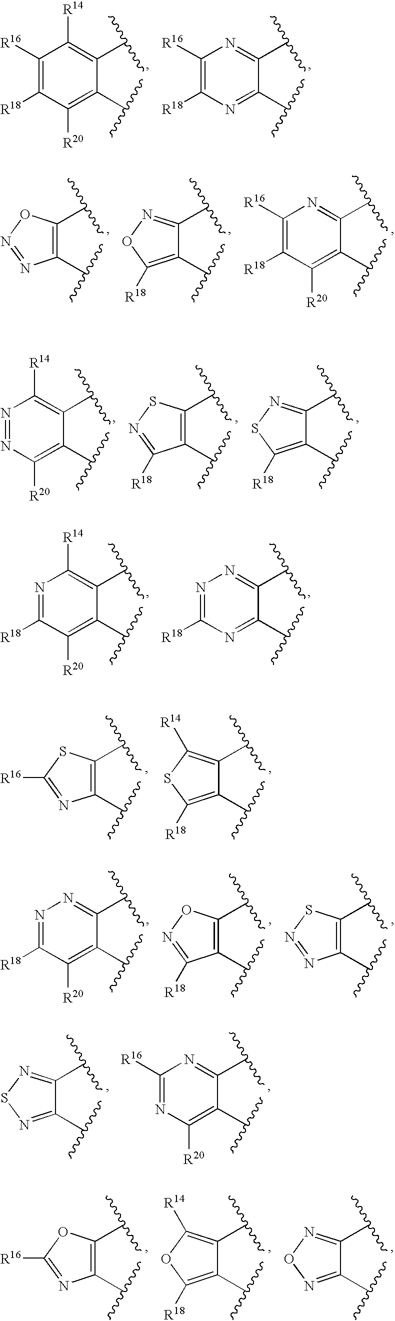

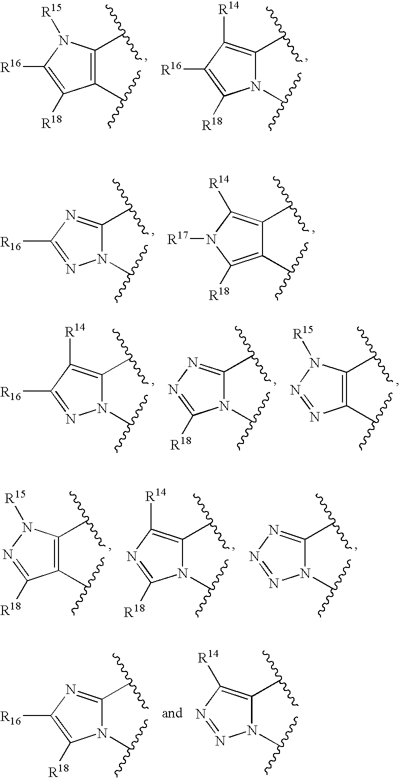

wherein the lines represent the bond in the parent structure;
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
R¹⁵ and R¹⁷, mutually independently, in each case denote H; —C(═O)—H; —C(═O)—R²⁷; —C(═O)—O—R²⁸; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
and R²², R²³, R²⁴, R²⁵, R²⁶, R²⁷, R²⁸, R²⁹, R³⁰, R³¹, R³², R³³, R³⁴, R³⁵, R³⁶ and R³⁷, mutually independently, in each case denote C₁₋₆ alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, pyrrolyl, indolyl, furanyl, benzo[b]furanyl, thiophenyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
with the proviso that compounds are excepted in which the sum of a, b, c, d, e and f is equal to 3 and W, M, P, Q, T and V together denote

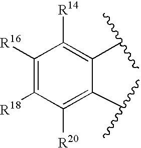

in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Propiolic acid amides which are also preferred are those of the above-stated general formula I, in which

R¹ denotes a residue selected from the group consisting of phenyl, thienyl, pyridazinyl, pyrazinyl, pyrimidinyl, pyridinyl, thiazolyl and thiadiazolyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C≡C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl;
a, b, c, d, e and f in each case denote 0 or 1; wherein the sum of a, b, c, d, e and f is equal to 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; —CF₃; —CF₂H; —CFH₂; —C₂F₅; —CH₂—CF₃; —CH₂—OH; —CH₂—NH₂; —CH₂—CN; —CH₂—CH₂—OH; —CH₂—CH₂—NH₂; —CH₂—CH₂—CN; —CH₂—CH₂—CH₂—OH; C₁₋₆ alkyl, C₂₋₆ alkenyl or C₂₋₆ alkynyl, which is in each case unsubstituted; C₂₋₆ heteroalkyl, C₂₋₆ heteroalkenyl or C₂₋₆ heteroalkynyl, which is in each case unsubstituted and/or comprises 1 or 2 heteroatom(s) mutually independently selected from the group consisting of oxygen, sulfur and nitrogen (NH) as chain link(s); or C₃₋₇ cycloalkyl, C₅₋₆ cycloalkenyl, 5- to 7-membered heterocycloalkyl and 5- to 7-membered heterocycloalkenyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
g denotes 0 or 1;
W, M, P, Q, T and optionally V together denote a ring selected from the group consisting of

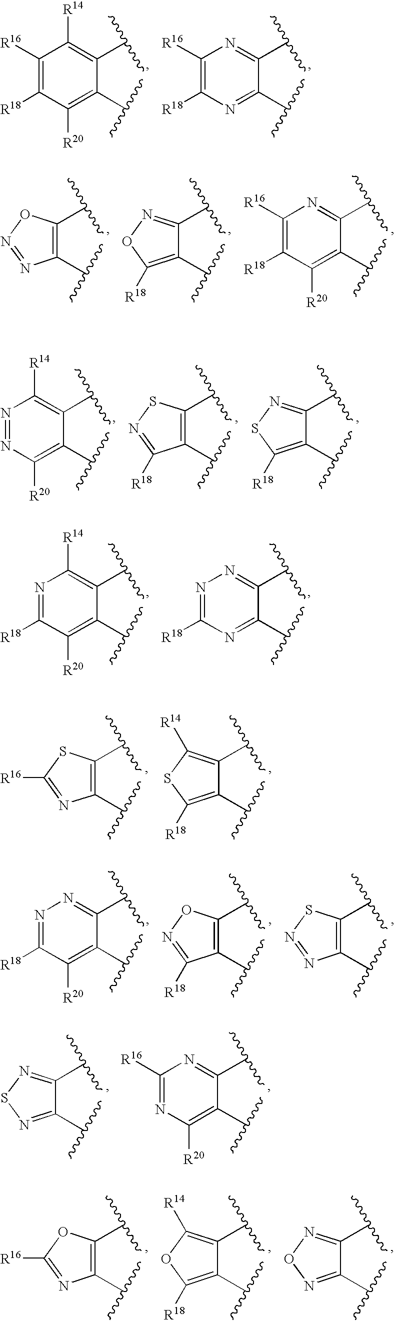

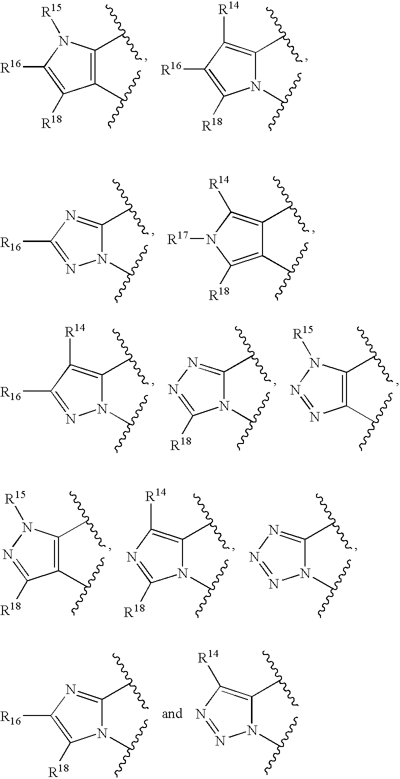

wherein the lines represent the bond in the parent structure;
R¹⁴, R¹⁶, R¹⁸ and R²⁰, mutually independently, in each case denote H; F; Cl; Br; I; —CF₃; —CF₂H; —CFH₂; —C₂F₅; —CH₂—CF₃; —NO₂; —CN; —NH₂; —OH; —SH; —C(═O)—OH; —C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted; C₃₋₇ cycloalkyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —O—CH₃, —O—C₂H₅ and —O—C₃H₇; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
R¹⁵ and R¹⁷, mutually independently, in each case denote H; —CF₃; —CF₂H; —CFH₂; —C₂F₅; —CH₂—CF₃; —C(═O)—H; —C(═O)—R²⁷; —C(═O)—O—R²⁸; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted; C₃₋₇ cycloalkyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —O—CH₃, —O—C₂H₅ and —O—C₃H₇; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —C—C₂H₅, —O—C₃H₇, —CF₃, —CHF₂, —CH₂F and —O—CF₃; and R²², R²³, R²⁴, R²⁵, R²⁶, R²⁷, R²⁸, R²⁹, R³⁰, R³¹, R³², R³³, R³⁴, R³⁵, R³⁶ and R³⁷ mutually independently, in each case denote —CF₃; —CF₂H; —CFH₂; —C₂F₅; —CH₂—CF₃; unsubstituted C₁₋₆ alkyl; C₃₋₇ cycloalkyl, C₅₋₆ cycloalkenyl, 5- to 7-membered heterocycloalkyl and 5- to 7-membered heterocycloalkenyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅ and —O—C₃H₇; or denote a residue selected from the group consisting of phenyl, naphthyl, furanyl, thiophenyl, pyrazolyl, imidazolyl, thiazolyl, thiadiazolyl, triazolyl, oxazolyl, oxadiazolyl and isoxazolyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
with the proviso that compounds are excepted in which the sum of a, b, c, d, e and f is equal to 3 and W, M, P, Q, T and V together denote

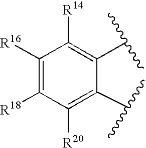

in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Very particularly preferred propiolic acid amides are those of the above-stated general formula I, in which

R¹ denotes a residue selected from the group consisting of phenyl, pyridinyl and thienyl, which is in each case unsubstituted or substituted with optionally 1, 2, 3, 4 or substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C≡C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl;
a, b, c, d, e and f in each case denote 0 or 1; wherein the sum of a, b, c, d, e and f is equal to 2, 3, 4 or 5;
R², R³, R⁴, R⁵, R⁶, R⁷, R⁸, R⁹, R¹⁰, R¹¹, R² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; —CF₃; —CF₂H; —CFH₂; —C₂F₅; —CH₂—CF₃; —CH₂—OH; —CH₂—NH₂; —CH₂—CN; —CH₂—CH₂—OH; —CH₂—CH₂—NH₂; —CH₂—CH₂—CN; —CH₂—CH₂—CH₂—OH; C₁₋₆ alkyl, C₂₋₆ alkenyl or C₂₋₆ alkynyl, which is in each case unsubstituted; or denote unsubstituted C₃₋₇ cycloalkyl;
g denotes 0 or 1;
W, M, P, Q, T and optionally V together denote a ring selected from the group consisting of

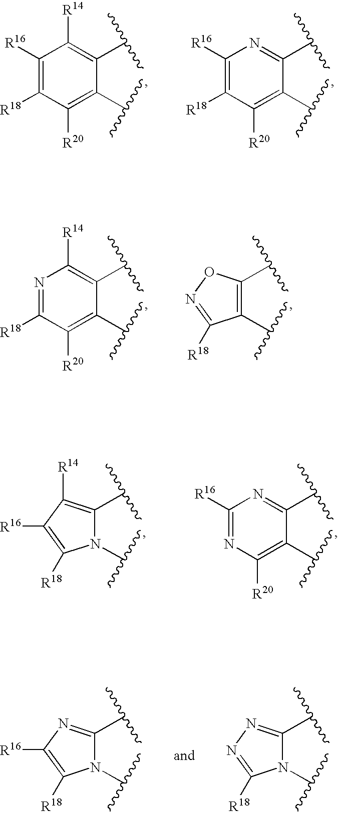

wherein the lines represent the bond in the parent structure;
R¹⁴, R¹⁶, R¹⁸ and R²⁰, mutually independently, in each case denote H; F; Cl; Br; I; —CF₃; —CF₂H; —CFH₂; —C₂F₅; —CH₂—CF₃; —NO₂; —CN; —NH₂; —OH; —C(═O)—OH; —C(═O)—H; —NH—R²²; —NR²³R²⁴; —O—R²⁵; —S—R²⁶; —C(═O)—R²⁷; —C(═O)—O—R²⁸—O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; C₁₋₆ alkyl, C₂₋₆ alkenyl or C₂₋₆ alkynyl, which is in each case unsubstituted; unsubstituted C₃₋₇ cycloalkyl; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
R²², R²³, R²⁴, R²⁷, R²⁸, R²⁹, R³⁰, R³¹, R³², R³³, R³⁴ and R³⁵, mutually independently, in each case denote —CF₃; —CF₂H; —CFH₂; —C₂F₅; —CH₂—CF₃; or denote unsubstituted C₁₋₆ alkyl;
and R²⁵ and R²⁶, mutually independently, in each case denote CF₃; —CF₂H; —CFH₂; —C₂F₅; —CH₂—CF₃; unsubstituted C₁₋₆ alkyl; or denote a residue selected from the group consisting of phenyl, naphthyl, furanyl, pyrazolyl, imidazolyl, thiazolyl, thiadiazolyl, triazolyl, oxazolyl, oxadiazolyl, isoxazolyl and thiophenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
with the proviso that compounds are excepted in which the sum of a, b, c, d, e and f is equal to 3 and W, M, P, Q, T and V together denote

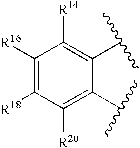

in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Propiolic acid amides which are also preferred are those of the above-stated general formula I, in which

R¹ denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
a, b, c, d, e and f in each case denote 0 or 1; wherein the sum of a, b, d and e is equal to 2, 3 or 4;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
g denotes 0 or 1;
W, M, P, Q, T and optionally V together denote a ring selected from the group consisting of

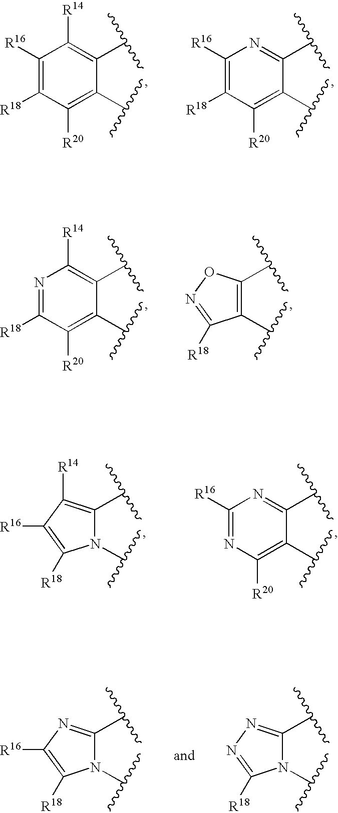

wherein the lines represent the bond in the parent structure;
and R¹⁴, R¹⁶, R¹⁸ and R²⁰, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
with the proviso that compounds are excepted in which the sum of a, b, c, d, e and f is equal to 3 and W, M, P, Q, T and V together denote;

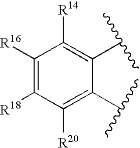

in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Propiolic acid amides which are still more preferred are those of the above-stated general formula I, in which

R¹ denotes a residue selected from the group consisting of phenyl, thienyl and pyridinyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
a, b, c, d, e and f in each case denote 0 or 1; wherein the sum of a, b, d and e is equal to 2, 3 or 4;
R² and R⁴, mutually independently, in each case denote H; methyl or ethyl;
R³, R⁵, R⁶, R⁷, R⁸, R⁹, R¹⁰, R¹¹, R¹² and R¹³ in each case denote H;
g denotes 0 or 1;
W, M, P, Q, T and optionally V together denote a ring selected from the group consisting of

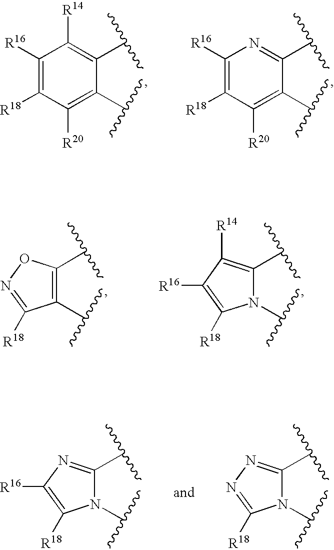

wherein the lines represent the bond in the parent structure;
R¹⁴, R¹⁶, R¹⁸ and R²⁰, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl;
with the proviso that compounds are excepted in which the sum of a, b, c, d, e and f is equal to 3 and W, M, P, Q, T and V together denote

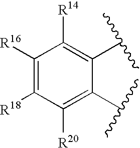

in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Very particularly preferred propiolic acid amides are those of the general formula Ia,

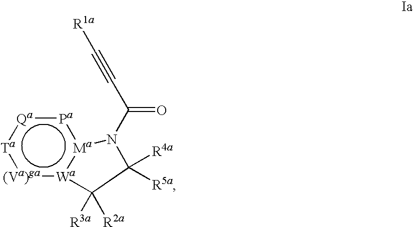

in which
R^{1a} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2a}, R^{3a}, R^{4a} and R^{5a}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
ga denotes 0 or 1;
W^{a}, M^{a}, P^{a}, Q^{a}, T^{a} and optionally V^{a} together denote a ring selected from the group consisting of

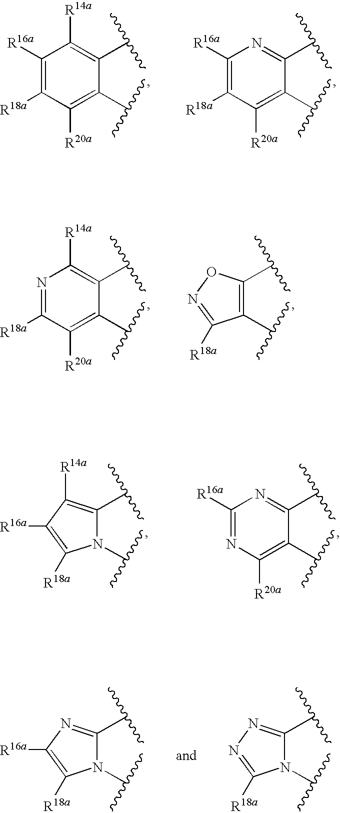

wherein the lines represent the bond in the parent structure; and R^{14a}, R^{16a}, R^{18a} and R^{20a}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Very particularly preferred propiolic acid amides are those of the general formula,

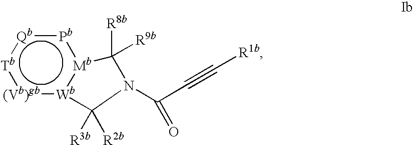

in which
R^{1b} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2b}, R^{3b}, R^{8b} and R^{9b}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
gb denotes 0 or 1;
W^{b}, M^{b}, P^{b}, Q^{b}, T^{b} and optionally V^{b} together denote a ring selected from the group consisting of

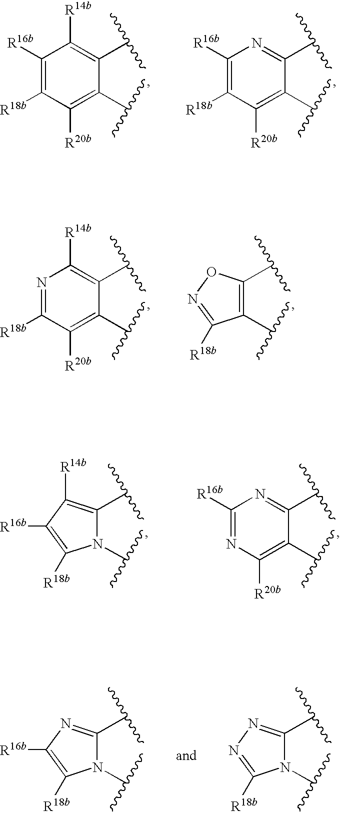

wherein the lines represent the bond in the parent structure;
and R^{14b}, R^{16b}, R^{18b} and R^{20b}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Very particularly preferred propiolic acid amides are those of the general formula Ic,

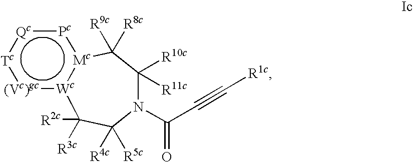

in which
R^{1c} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2c}, R^{3c}, R^{4c}, R^{5c}, R^{8c}, R^{9c}, R^{10c} and R^{11c}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
gc denotes 0 or 1;
W^{c}, M^{c}, P^{c}, Q^{c}, T^{c} and optionally V^{c} together denote a ring selected from the group consisting of

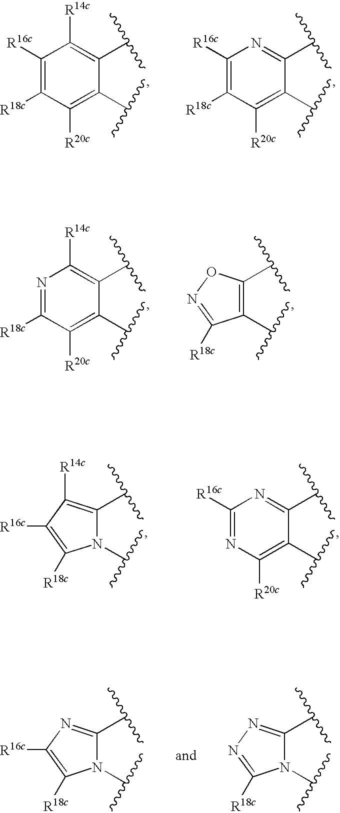

wherein the lines represent the bond in the parent structure;
and R^{14c}, R^{16c}, R^{18c} and R^{20c}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Very particularly preferred propiolic acid amides are those of the general formula Id,

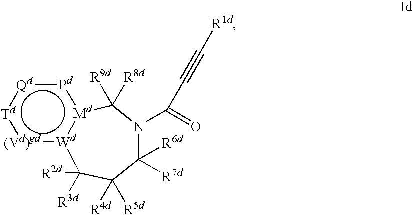

in which
R^{1d} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2d}, R^{3d}, R^{4d}, R^{5d}, R^{6d}, R^{7d}, R^{8d} and R^{9d}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
gd denotes 0 or 1;
W^{d}, M^{d}, P^{d}, Q^{d}, T^{d} and optionally V^{d} together denote a ring selected from the group consisting of

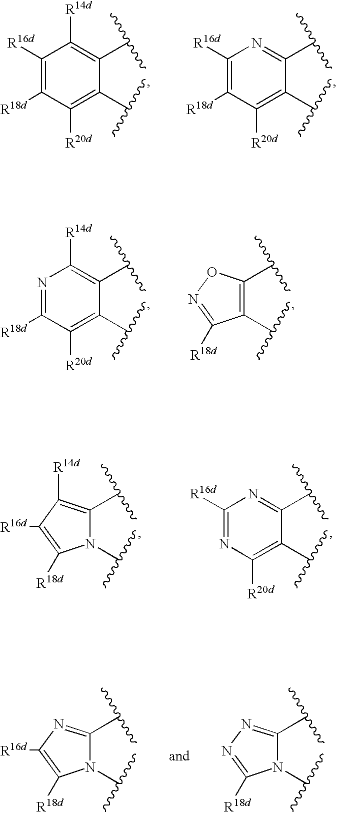

wherein the lines represent the bond in the parent structure;
and R^{14d}, R^{16d}, R^{18d} and R^{20d}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Substituted propiolic acid amides of the above-stated general formula I which are still more preferred are those selected from the group consisting of
[3] 1-(3-phenyl-propiolyl)-2,3-dihydro-indole,
[4] 6-(3-phenyl-propiolyl)-5,6,7,8-tetrahydro-[1,6]naphthyridine,
[6] 2-(3-phenyl-propiolyl)-2,3-dihydro-isoindole,
[8] 3-methyl-5-(3-phenyl-propiolyl)-4,5,6,7-tetrahydro-isoxazolo[4,5-c]pyridine,
[9] 3-phenyl-5-(3-phenyl-propiolyl)-4,5,6,7-tetrahydro-isoxazolo[4,5-c]pyridine,
[10] 7-(3-phenyl-propiolyl)-5,6,7,8-tetrahydro-[1,2,4]triazolo[4,3-a]pyrazine,
[12] 3-(3-(3-chlorophenyl)-propiolyl)-1,2,4,5-tetrahydrobenzo[d]azepine,
[21] 2-(3-(3-chlorophenyl)-propiolyl)-2,3,4,5-tetrahydro-1H-benzo[c]azepine,
[23] 7-(3-(3-chlorophenyl)-propiolyl)-5,6,7,8-tetrahydroimidazo[1,2-a]pyrazine,
[27] 2-(3-(3-chlorophenyl)-propiolyl)-1,2,3,4-tetrahydropyrrolo[1,2-a]pyrazine,
[33] 1-(3-(2,4-difluorophenyl)-propiolyl)-2-methyl-indoline,
[36] 1-(3-(4-fluoro-3-methylphenyl)-propiolyl)-2-methyl-indoline,
[38] 1-(3-(2-fluorophenyl)-propiolyl)-2-methyl-indoline,
[40] 1-(3-(4-tolyl)-propiolyl)-2-methyl-indoline,
[42] 1-(3-(4-trifluoromethylphenyl)-propiolyl)-2-methyl-indoline,
[44] 1-(3-phenyl-propiolyl)-2-methyl-indoline,
[46] 1-(3-(2,3-dimethylphenyl)-propiolyl)-2-methyl-indoline,
[48] 1-(3-(3,5-dimethylphenyl)-propiolyl)-2-methyl-indoline,
[50] 1-(3-(2,4-dichlorophenyl)-propiolyl)-2-methyl-indoline,
[52] 1-(3-(3-tolyl)-propiolyl)-2-methyl-indoline,
[54] 1-(3-(4-methoxyphenyl)-propiolyl)-2-methyl-indoline,
[56] 1-(3-(2-thienyl)-propiolyl)-2-methyl-indoline,
[63] 1-(3-(2,4-dimethylphenyl)-propiolyl)-2-methyl-indoline,
[65] 1-(3-(4-fluorophenyl)-propiolyl)-2-methyl-indoline,
[66] 1-(3-(3-chlorophenyl)-propiolyl)-2-methyl-indoline,
[68] 1-(3-(3-fluoro-4-methylphenyl)-propiolyl)-2-methyl-indoline,
[70] 1-(3-(4-tert.-butylphenyl)-propiolyl)-2-methyl-indoline,
[72] 1-(3-(3,4-dimethylphenyl)-propiolyl)-2-methyl-indoline,
[73] 1-(3-(2-bromo-5-methoxyphenyl)-propiolyl)-2-methyl-indoline,
[74] 1-(3-(2-tolyl)-propiolyl)-2-methyl-indoline and
[75] 1-(3-(2-trifluoromethylphenyl)-propiolyl)-2-methyl-indoline;
   in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates.

Particular preference is also given to substituted propiolic acid amides of the above-stated general formula I, which after 60 minutes' incubation in 450 pg of protein from pig brain homogenate at a temperature of between 20° C. and 25° C. in a concentration of less than 2000 nM, preferably less than 1000 nM, particularly preferably less than 700 nM, very particularly preferably less than 100 nM, still more preferably less than 30 nM, bring about a 50 percent displacement of [³H]-2-methyl-6-(3-methoxyphenyl)-ethynylpyridine, which is present in a concentration of 5 nM.

The displacement of [³H]-2-methyl-6-(3-methoxyphenyl)-ethynylpyridine is here determined as described in the section Pharmacological methods, method I for determining the inhibition of [³H]-MPEP-binding in the mGluR5 receptor binding assay.

The present invention also provides a method for producing compounds of the above-stated general formula I, in accordance with which at least one compound of the general formula II,

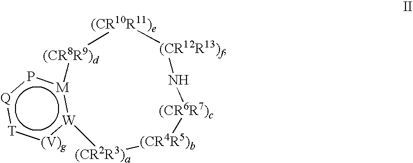

in which R², R³, R⁴, R⁵, R⁶, R⁷, R⁸, R⁹, R¹⁰, R¹¹, R¹², R¹³, M, W, P, Q, T, V, a, b, c, d, e, f and g have the above-stated meaning, by reaction with at least one compound of the general formula R¹—C≡C—C(═O)—OH, in which R¹ has the above-stated meaning, optionally in a reaction medium, optionally in the presence of at least one suitable coupling agent, optionally in the presence of at least one base, preferably at a temperature of −70° C. to 100° C., or by reaction with at least one compound of the general formula R¹—C≡C—C(═O)—X, in which R¹ has the above-stated meaning and X denotes a leaving group, preferably denotes a leaving group selected from the group consisting of halogen residues, imidazol-1-yl and pentafluorophenoxy, particularly preferably denotes a chlorine residue, in a reaction medium, optionally in the presence of at least one base, preferably at a temperature of −70° C. to 100° C., is converted into at least one corresponding compound of the general formula I, optionally in the form of a corresponding salt,

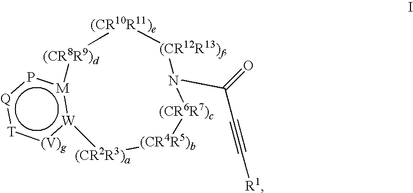

in which R¹, R², R³, R⁴, R⁵, R⁶, R⁷, R⁸, R⁹, R¹⁰, R¹¹, R¹², R¹³, W, P, Q, T, V, a, b, c, d, e, f and g have the above-stated meaning, and this compound is optionally purified and/or isolated;
or at least one compound of the general formula II, by reaction with propiolic acid [HC≡C—C(═O)—OH] optionally in a reaction medium, optionally in the presence of at least one suitable coupling agent, optionally in the presence of at least one base, preferably at a temperature of −70° C. to 100° C., or by reaction with at least one compound of the general formula HC≡C—C(═O)—X, in which X denotes a leaving group, preferably denotes a leaving group selected from the group consisting of halogen residues, imidazol-1-yl and pentafluorophenoxy, particularly preferably denotes a chlorine residue, in a reaction medium, optionally in the presence of at least one base, preferably at a temperature of −70° C. to 100° C., is converted into at least one corresponding compound of the general formula III, optionally in the form of a corresponding salt,

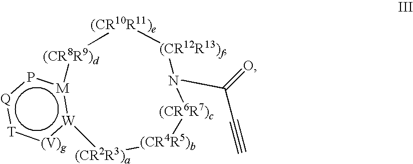

in which R², R³, R⁴, R⁵, R⁶, R⁷, R⁸, R⁹, R¹⁰, R¹¹, R¹², R¹³, M, W, P, Q, T, V, a, b, c, d, e, f and g have the above-stated meaning, and this compound is optionally purified and/or isolated,
and at least one compound of the general formula III, by reaction with at least one compound of the general formula R¹—X, in which R¹ has the above-stated meaning and X denotes a leaving group, preferably denotes a halogen residue or a sulfonic acid ester, particularly preferably iodine, bromine or triflate, optionally in a reaction medium, optionally in the presence of at least one catalyst, preferably in the presence of at least one palladium catalyst selected from the group consisting of palladium chloride [PdCl₂], palladium acetate [Pd(OAc)₂], tetrakistriphenylphosphinepalladium [Pd(PPh₃)₄], bistriphenylphosphinepalladium dichloride [Pd(PPh₃)₂Cl₂] and bistriphenylphosphinepalladium acetate [Pd(PPh₃)₂(OAc)₂], optionally in the presence of at least one ligand, preferably in the presence of at least one ligand selected from the group consisting of triphenylphosphine, triphenylarsine and tri-2-furyl-phosphine, optionally in the presence of at least one inorganic salt, preferably in the presence of at least one inorganic salt selected from the group consisting of lithium chloride and zinc chloride, optionally in the presence of at least one copper salt, preferably in the presence of copper iodide, optionally in the presence of at least one organic or inorganic base, preferably in the presence of at least one base selected from the group consisting of triethylamine, [1,4]-diazabicyclo-[2.2.2]-octane, diisopropylamine, diisopropylethylamine, potassium carbonate and sodium hydrogencarbonate, preferably at a temperature of between −70° C. and 300° C., is converted into at least one corresponding compound of the general formula I, optionally in the form of a corresponding salt, and this compound is optionally purified and/or isolated.

A method according to the invention for producing substituted propiolic acid amides of the above-stated general formula I is also stated in Scheme 1 below.

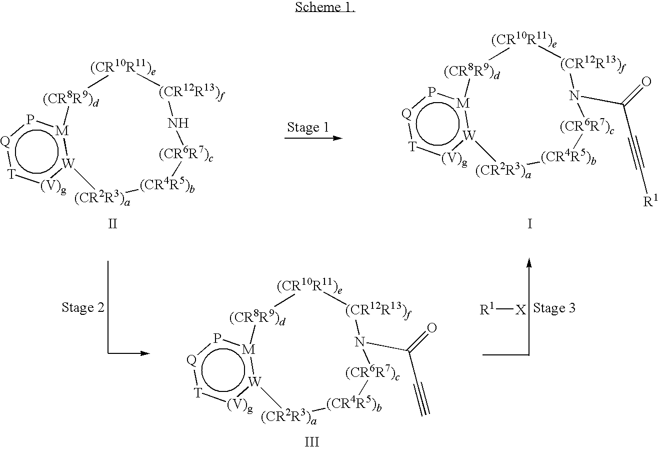

In stage 1, compounds of the above-stated general formula II are reacted with carboxylic acids of the above-stated general formula R¹—C≡C—C(═O)—OH in a reaction medium, preferably selected from the group consisting of diethyl ether, tetrahydrofuran, acetonitrile, methanol, ethanol, (1,2)-dichloroethane, dimethylformamide, dichloromethane and corresponding mixtures, optionally in the presence of at least one coupling agent, preferably selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCl), PL-EDC (polymer-bound N-benzyl-3-((ethylimino)methyleneamino)-N,N-dimethylpropane-1-aminium chloride), 1,1′-carbonyl-diimidazole (CDI), N-[(dimethylamino)-1H-1,2,3-triazolo[4,5-b]pyridino-1-ylmethylene]-N-methylmethaneaminium hexafluorophosphate N-oxide (HATU), O-(benzotriazol-1-yl)-N,N,N′,N′-tetramethyluronium hexafluorophosphate (HBTU), O-(benzotriazol-1-yl)-N,N,N′,N′-tetramethyluronium tetrafluoroborate (TBTU), 1-hydroxy-7-azabenzotriazole (HOAt) and polymer-bound carbodiimide resin (PS-carbodiimide resin, PSii), particularly preferably in the presence of a coupling agent selected from the group consisting of TBTU, EDCl and PL-EDC, optionally in the presence of at least one organic base, preferably selected from the group consisting of triethylamine, pyridine, dimethylaminopyridine, N-methylmorpholine, [1,4]-diazabicyclo-[2.2.2]-octane (DABCO), 1,8-diazabicyclo[5.4.0]undec-7-ene (DBU), 1,5-diazabicyclo[4.3.0]non-5-ene (DBN) and diisopropylethylamine, preferably in the presence of diisopropylethylamine or triethylamine, preferably at temperatures of −70° C. to 250° C. to yield compounds of the general formula I.

Alternatively, compounds of the above-stated general formula II are reacted with carboxylic acid derivatives of the above-stated general formula R¹—C≡C—C(═O)—X, in which X denotes a leaving group, preferably denotes a leaving group selected from the group consisting of halogen residues, imidazol-1-yl and pentafluorophenoxy, particularly preferably chlorine or bromine, in a reaction medium, preferably selected from the group consisting of diethyl ether, tetrahydrofuran, acetonitrile, methanol, ethanol, dimethylformamide, dichloromethane, 1,2-dichloroethane and corresponding mixtures, optionally in the presence of an organic base or inorganic base, preferably selected from the group consisting of triethylamine, dimethylaminopyridine, pyridine and diisopropylamine, at temperatures of −70° C. to 250° C. to yield compounds of the general formula I.

In stage 2, compounds of the above-stated general formula II are reacted with propiolic acid H—C≡C—C(═O)—OH or with carboxylic acid derivatives of the general formula H—C≡C—C(═O)—X, in which X denotes a leaving group, preferably a halogen residue, particularly preferably chlorine or bromine, as described in scheme 1, stage 1, to yield compounds of the general formula III.

In stage 3, compounds of the above-stated general formula III are reacted with compounds of the general formula R¹—X, in which R¹ has the above-stated meaning and X denotes a leaving group, preferably denotes a halogen residue or a sulfonic acid ester, particularly preferably iodine, bromine or triflate, in a reaction medium, preferably in a reaction medium selected from the group consisting of methanol, ethyl acetate, ethanol, isopropanol, n-butanol, diethyl ether, dioxane, tetrahydrofuran, chloroform, dichloromethane, dimethylformamide, acetonitrile, pyridine, dimethyl sulfoxide, water, toluene and corresponding mixtures, preferably in dimethylformamide, water, ethyl acetate, tetrahydrofuran and corresponding mixtures, optionally in the presence of at least one catalyst, preferably in the presence of a palladium catalyst selected from the group consisting of palladium chloride [PdCl₂], palladium acetate [Pd(OAc)₂], tetrakistriphenylphosphinepalladium [Pd(PPh₃)₄], bistriphenylphosphinepalladium dichloride [Pd(PPh₃)₂Cl₂] and bistriphenylphosphinepalladium acetate [Pd(PPh₃)₂(OAc)₂], preferably in the presence of Pd(PPh₃)₄, Pd(PPh₃)₂Cl₂ and Pd(PPh₃)₂(OAc)₂, optionally in the presence of at least one ligand, preferably in the presence of at least one of a ligand selected from the group consisting of triphenylphosphine, triphenylarsine and tri-2-furyl-phosphine, preferably in the presence of triphenylphosphine, optionally in the presence of at least one inorganic salt, preferably in the presence of at least one inorganic salt selected from the group consisting of lithium chloride and zinc chloride, optionally in the presence of at least one copper salt, preferably in the presence of copper iodide, optionally in the presence of at least one organic or inorganic base, preferably in the presence of at least one base selected from the group consisting of triethylamine, [1,4]-diazabicyclo-[2.2.2]-octane, diisopropylamine, diisopropylethylamine, potassium carbonate and sodium hydrogencarbonate, preferably at a temperature of between −70° C. and 250° C. to yield a compound of the general formula I. Particularly preferably, compounds of the general formula R¹—I, or R¹—Br are reacted with compounds of the general formula III in a reaction medium selected from the group consisting of dimethylformamide or ethyl acetate in the presence of Pd(PPh₃)₂Cl₂, copper(I) iodide and a base selected from the group consisting of diisopropylamine or triethylamine.

The compounds the above-stated formula II, and of the general formulae R¹—C≡C—C(═O)—OH, R¹—C≡C—C(═O)—X, R¹—X and H—C≡C—C(═O)—X, are in each case commercially available and/or may be produced using conventional methods known to a person skilled in the art.

The above-described reactions may in each case be performed under conventional conditions familiar to a person skilled in the art, for example with regard to pressure or the sequence of addition of the components. Optimum control of the method according to the respective conditions may optionally be established by a person skilled in the art by simple preliminary testing.

The intermediate and final products obtained from the above-described reactions may in each case, if desired and/or necessary, be purified and/or isolated using conventional methods known to a person skilled in the art. Suitable purification methods are, for example, extraction methods and chromatographic methods such as column chromatography or preparative chromatography.

All the above-described method steps and in each case also the purification and/or isolation of intermediate or final products may be performed in part or entirely under an inert gas atmosphere, preferably under a nitrogen atmosphere.

If the substituted propiolic acid amides according to the invention of the above-stated general formulae I, Ia, Ib, Ic, and Id or of the general formulae Ie, Ie1, If1, Ig, Ih, and If, hereinafter described as substituted propiolic acid amides of the general formula I, are obtained after their production in the form of a mixture of the stereoisomers, preferably in the form of the racemates thereof or other mixtures of the various enantiomers and/or diastereomers thereof, these may be separated and optionally isolated using conventional methods known to a person skilled in the art. Examples which may be mentioned are chromatographic separation methods, in particular liquid chromatography methods at standard pressure or at elevated pressure, preferably MPLC and HPLC methods, and fractional crystallisation methods. Individual enantiomers, e.g. diastereomeric salts formed by means of HPLC on a chiral phase or by means of crystallisation with chiral acids, for instance (+)-tartaric acid, (−)-tartaric acid or (+)-10-camphorsulfonic acid, may here in particular be separated from one another.

The substituted propiolic acid amides according to the invention of the above-stated general formula I and optionally in each case corresponding stereoisomers may be obtained using conventional methods known to a person skilled in the art in the form of corresponding salts, preferably in the form of corresponding hydrochlorides, in particular in the form of corresponding physiologically acceptable salts, wherein the medicament according to the invention may comprise one or more salts of one or more of these compounds.

The respective salts of the substituted propiolic acid amides according to the invention of the above-stated general formula I and corresponding stereoisomers may be obtained for example by reaction with one or more inorganic acids and/or one or more organic acids. Suitable acids may preferably be selected from the group consisting of perchloric acid, hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid, saccharic acid, cyclohexanesulfamic acid, aspartame, monomethylsebacic acid, 5-oxo-proline, hexane-1-sulfonic acid, nicotinic acid, 2-aminobenzoic acid, 3-aminobenzoic acid or 4-aminobenzoic acid, 2,4,6-trimethylbenzoic acid, α-lipoic acid, acetylglycine, hippuric acid, phosphoric acid, maleic acid, malonic acid and aspartic acid.

The substituted propiolic acid amides according to the invention of the above-stated general formula I, and optionally corresponding stereoisomers and in each case the physiologically acceptable salts thereof may be obtained using conventional methods known to a person skilled in the art also in the form of the solvates thereof, in particular in the form of the hydrates thereof.

It has surprisingly been found that the above-stated substituted propiolic acid amides of the general formula I are suitable for mGluR5 receptor regulation and may therefore be used in particular as pharmaceutical active ingredients in medicaments for the prevention and/or treatment of disorders or diseases associated with these receptors or processes.

The substituted propiolic acid amides according to the invention of the above-stated general formula I, including above-excepted compounds and optionally corresponding stereoisomers and in each case the corresponding salts and solvates appear to be toxicologically safe and are therefore suitable as pharmaceutically active ingredients in pharmaceutical preparations.

The present invention accordingly also provides a medicament containing at least one substituted propiolic acid amide according to the invention of the above-stated general formula I, including above-excepted compounds, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances.

The present invention also provides a medicament containing at least one substituted propiolic acid amide according to the invention of the general formula Ie,

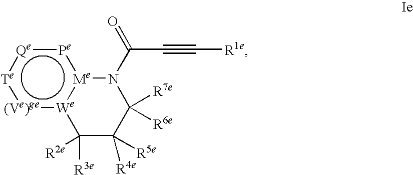

in which
R^{1e} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2e}, R^{3e}, R^{4e}, R^{5e}, R^{6e} and R^{7e}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
ge denotes 0 or 1;
W^{e}, M^{e}, P^{e}, Q^{e}, T^{e} and optionally V^{e} together denote a ring selected from the group consisting of

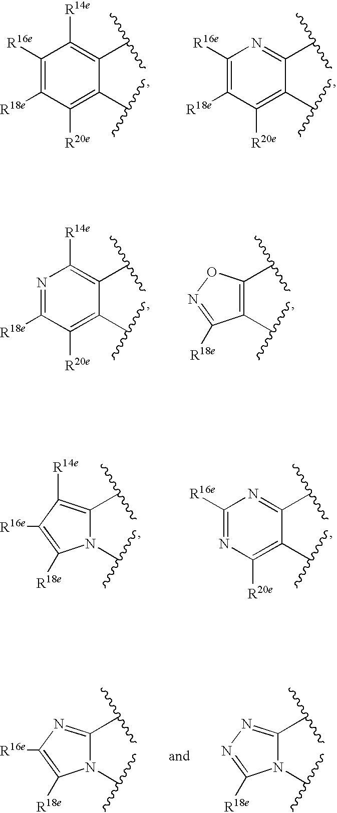

wherein the lines represent the bond in the parent structure;
and R^{14e}, R^{16e}, R^{18e} and R^{20e}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates, and optionally one or more pharmaceutically acceptable auxiliary substances.

The present invention also provides a medicament containing at least one substituted propiolic acid amide according to the invention of the general formula Ie1,

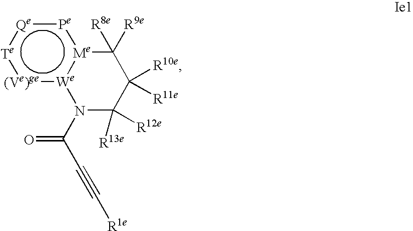

in which W^{e}, M^{e}, P^{e}, Q^{e}, T^{e}, V^{e}, ge, R^{1e}, R^{8e}, R^{9e}, R^{10e}, R^{11e}, R^{12e} and R^{13e} have the above-stated meaning.

The present invention also provides a medicament containing at least one substituted 1-propiolyl-1,2,3,4-tetrahydro-quinoline according to the invention of the general formula Ig,

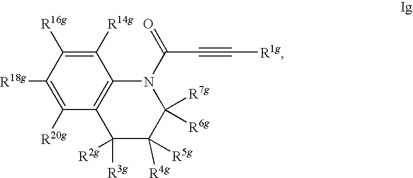

in which
R^{1g} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2g}, R^{3g}, R^{4g}, R^{5g}, R^{6g} and R^{7g}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
and R^{14g}, R^{16g}, R^{18g} and R^{20g}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates, and optionally one or more pharmaceutically acceptable auxiliary substances.

The present invention also provides a medicament containing at least one substituted propiolic acid amide according to the invention of the general formula If,

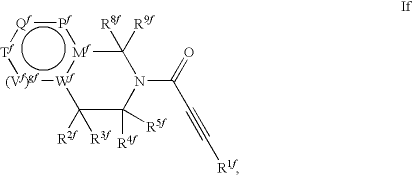

in which
R^{1f} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2f}, R^{3f}, R^{4f}, R^{5f}, R^{8f} and R^{9f}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
gf denotes 0 or 1;
W^{f}, M^{f}, P^{f}, Q^{f}, T^{f} and optionally V^{f} together denote a ring selected from the group consisting of

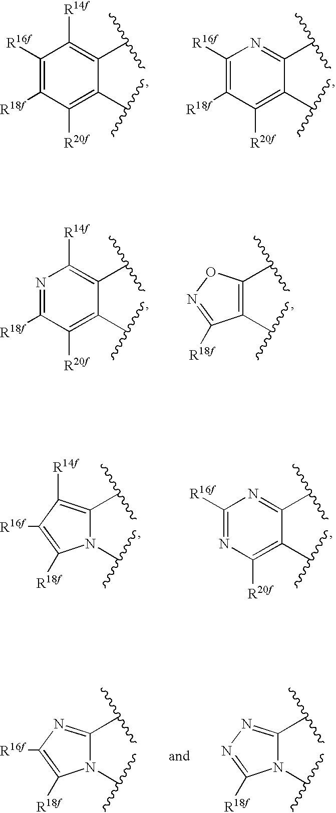

wherein the lines represent the bond in the parent structure;
and R^{14f}, R^{16f}, R^{18f} and R^{20f}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates, and optionally one or more pharmaceutically acceptable auxiliary substances.

The present invention also provides a medicament containing at least one substituted propiolic acid amide according to the invention of the general formula If1,

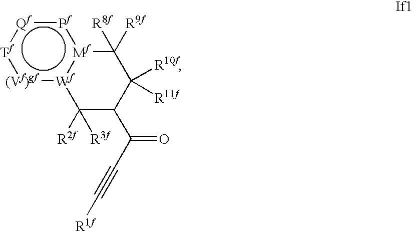

in which W^{f}, M^{f}, P^{f}, Q^{f}, T^{f}, V^{f}, gf, R^{1f}, R^{2f}, R^{3f}, R^{8f}, R^{9f}, R^{10f} and R^{11f} have the above-stated meaning.

The present invention also provides a medicament containing at least one substituted 2-propiolyl-1,2,3,4-tetrahydro-isoquinoline according to the invention of the general formula Ih,

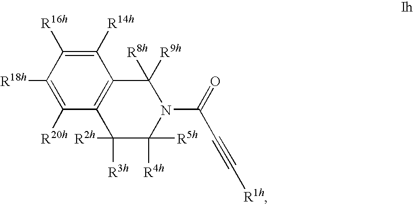

in which
R^{1h} denotes a residue selected from the group consisting of phenyl, pyridinyl and thienyl, which is in each case unsubstituted or is substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2h}, R^{3h}, R^{4h}, R^{5h}, R^{8h} and R^{9h}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
and R^{14g}, R^{16g}, R^{18g} and R^{20g}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates, and optionally one or more pharmaceutically acceptable auxiliary substances.

The present invention also provides a medicament containing at least one substituted propiolic acid amide according to the invention selected from the group consisting of
[1] 2-(3-phenyl-propiolyl)-1,2,3,4-tetrahydro-isoquinoline,
[2] 1-(3-phenyl-propiolyl)-1,2,3,4-tetrahydro-quinoline,
[5] 2-(3-(3-chlorophenyl)-propiolyl)-1,2,3,4-tetrahydro-isoquinoline,
[7] 2-(3-pyrid-2-yl-propiolyl)-1,2,3,4-tetrahydro-isoquinoline,
[11] 2-(3-(3-chlorophenyl)-propiolyl)-6,7-dimethoxy-1,2,3,4-tetrahydro-isoquinoline,
[13] 2-(3-(3-chlorophenyl)-propiolyl)-6,7-dihydroxy-1,2,3,4-tetrahydro-isoquinoline,
[14] 6-methoxy-2-(3-phenyl-propiolyl)-1,2,3,4-tetrahydro-isoquinoline,
[15] 2-(3-(3-chlorophenyl)-propiolyl)-7-hydroxy-6-methoxy-1,2,3,4-tetrahydro-isoquinoline,
[16] 2-(3-(3-chlorophenyl)-propiolyl)-6-hydroxy-7-methoxy-1,2,3,4-tetrahydro-isoquinoline,
[17] 2-(3-(3-chlorophenyl)-propiolyl)-6-methoxy-1,2,3,4-tetrahydro-isoquinoline,
[18] 2-(3-(3-chlorophenyl)-propiolyl)-1-methyl-1,2,3,4-tetrahydro-isoquinoline,
[19] 2-(3-(3-chlorophenyl)-propiolyl)-3-methyl-1,2,3,4-tetrahydro-isoquinoline,
[20] 2-(3-(3-chlorophenyl)-propiolyl)-6-methyl-1,2,3,4-tetrahydro-isoquinoline,
[22] 2-(3-(3-chlorophenyl)-propiolyl)-7-methoxy-1,2,3,4-tetrahydro-isoquinoline,
2-(3-(3-chlorophenyl)-propiolyl)-8-methyl-1,2,3,4-tetrahydro-isoquinoline,
2-(3-(3-chlorophenyl)-propiolyl)-7-methyl-1,2,3,4-tetrahydro-isoquinoline,
2-(3-(3-chlorophenyl)-propiolyl)-5-methyl-1,2,3,4-tetrahydro-isoquinoline,
2-(3-(3-chlorophenyl)-propiolyl)-7-hydroxy-1,2,3,4-tetrahydro-isoquinoline,
2-(3-(3-chlorophenyl)-propiolyl)-8-methoxy-1,2,3,4-tetrahydro-isoquinoline,
2-(3-(3-chlorophenyl)-propiolyl)-5-methoxy-1,2,3,4-tetrahydro-isoquinoline,
2-(3-(3-chlorophenyl)-propiolyl)-1,8-dimethyl-1,2,3,4-tetrahydro-isoquinoline,
2-(3-(2,4-difluorophenyl)-propiolyl)-1,2,3,4-tetrahydro-isoquinoline,
[34] 2-(3-(3-methoxyphenyl)-propiolyl)-1,2,3,4-tetrahydro-isoquinoline,
[35] 2-(3-(4-fluoro-3-methyl-phenyl)-propiolyl)-1,2,3,4-tetrahydro-isoquinoline,
[37] 2-(3-(2-fluorophenyl)-propiolyl)-1,2,3,4-tetrahydro-isoquinoline,
[39] 2-(3-(4-tolyl)-propiolyl)-1,2,3,4-tetrahydro-isoquinoline,
[41] 2-(3-(4-trifluoromethylphenyl)-propiolyl)-1,2,3,4-tetrahydro-isoquinoline,
[43] 2-(3-phenyl-propiolyl)-1,2,3,4-tetrahydro-isoquinoline,
[45] 2-(3-(2,3-dimethylphenyl)-propiolyl)-1,2,3,4-tetrahydro-isoquinoline,
[47] 2-(3-(3,5-dimethylphenyl)-propiolyl)-1,2,3,4-tetrahydro-isoquinoline,
[49] 2-(3-(2,4-dichlorophenyl)-propiolyl)-1,2,3,4-tetrahydro-isoquinoline,
[51] 2-(3-(3-tolyl)-propiolyl)-1,2,3,4-tetrahydro-isoquinoline,
[53] 2-(3-(4-methoxyphenyl)-propiolyl)-1,2,3,4-tetrahydro-isoquinoline,
[55] 2-(3-(2-thienyl)-propiolyl)-1,2,3,4-tetrahydro-isoquinoline,
[57] 1-(3-(4-tolyl)-propiolyl)-1,2,3,4-tetrahydro-quinoline,
[58] 1-(3-(4-trifluoromethylphenyl)-propiolyl)-1,2,3,4-tetrahydro-quinoline,
[59] 1-(3-(2,3-dimethylphenyl)-propiolyl)-1,2,3,4-tetrahydro-quinoline,
[60] 1-(3-(2,4-dichlorophenyl)-propiolyl)-1,2,3,4-tetrahydro-quinoline,
[61] 1-(3-(2,4-dimethylphenyl)-propiolyl)-1,2,3,4-tetrahydro-quinoline,
[62] 1-(3-(4-tert.-butylphenyl)-propiolyl)-1,2,3,4-tetrahydro-quinoline,
[64] 2-(3-(4-fluorophenyl)-propiolyl)-1,2,3,4-tetrahydro-isoquinoline,
[67] 2-(3-(3-fluoro-4-methyl-phenyl)-propiolyl)-1,2,3,4-tetrahydro-isoquinoline,
[69] 2-(3-(4-tert.-butylphenyl)-propiolyl)-1,2,3,4-tetrahydro-isoquinoline,
[71] 2-(3-(3,4-dimethylphenyl)-propiolyl)-1,2,3,4-tetrahydro-isoquinoline,
[76] 2-(3-(2-cyano-phenyl)-propiolyl)-1,2,3,4-tetrahydro-isoquinoline,
[77] 2-(3-(3-cyano-phenyl)-propiolyl)-1,2,3,4-tetrahydro-isoquinoline,
[78] 1-(3-(2-tolyl)-propiolyl)-1,2,3,4-tetrahydro-quinoline and
[79] 1-(3-(2-trifluoromethylphenyl)-propiolyl)-1,2,3,4-tetrahydro-quinoline;
   in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of corresponding salts or in each case in the form of corresponding solvates, and optionally one or more pharmaceutically acceptable auxiliary substances.

The medicament according to the invention is suitable for mGluR5 receptor regulation, in particular for inhibiting the mGluR5 receptor.

Preferably, the medicament according to the invention is suitable for the prevention and/or treatment of disorders and/or diseases which are at least partially mediated by mGluR5 receptors.

The medicament according to the invention is therefore particularly preferably suitable for the treatment and/or prevention of pain, preferably of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's chorea; cognitive diseases, preferably cognitive deficiency states, particularly preferably attention deficit syndrome (ADS); anxiety states; panic attacks; epilepsy; coughing; urinary incontinence; diarrhoea; pruritus; schizophrenia; cerebral ischaemic episodes; muscle spasms; cramps; pulmonary diseases, preferably selected from the group comprising asthma and pseudocroup; regurgitation (vomiting); stroke; dyskinesia; retinopathy; lack of drive; laryngitis; disorders of food intake, preferably selected from the group consisting of bulimia, cachexia, anorexia and obesity; dependency on alcohol; dependency on medicines; dependency on drugs, preferably nicotine- and/or cocaine-dependency; alcohol abuse; abuse of medicines; drug abuse; preferably nicotine and/or cocaine abuse; withdrawal symptoms associated with dependency on alcohol, medicines and/or drugs (in particular nicotine- and/or cocaine-dependency); development of tolerance towards medicines, preferably towards natural or synthetic opioids; gastro-oesophageal reflux syndrome; gastro-oesophageal reflux disease; irritable bowel syndrome; for diuresis; for antinatriuresis; for influencing the cardiovascular system; for increasing vigilance; for increasing libido; for modulating locomotor activity or for local anaesthesia.

The medicament according to the invention is very particularly preferably suitable for the prevention of pain, preferably of pain selected from the group consisting of acute pain, chronic pain, neuropathic pain and visceral pain; anxiety states; panic attacks; dependency on alcohol; dependency on medicines; disorders of food intake, preferably selected from the group consisting of bulimia, cachexia, anorexia and obesity; dependency on drugs, preferably nicotine- and/or cocaine-dependency; alcohol abuse; abuse of medicines; drug abuse; preferably nicotine and/or cocaine abuse; withdrawal symptoms associated with dependency on alcohol, medicines and/or drugs (in particular nicotine- and/or cocaine-dependency); development of tolerance towards medicines and/or drugs, preferably towards natural or synthetic opioids; gastro-oesophageal reflux syndrome, gastro-oesophageal reflux disease and irritable bowel syndrome.

The medicament according to the invention is still more preferably suitable for the prevention and/or treatment of pain, preferably of pain selected from the group consisting of acute pain, chronic pain, neuropathic pain and visceral pain, anxiety states and panic attacks.

The medicament according to the invention is most preferably suitable for the prevention and/or treatment of pain, preferably acute pain, chronic pain, neuropathic pain or visceral pain.

The present invention also provides the use of at least one substituted propiolic acid amide according to the invention of the above-stated general formula I, including above-excepted compounds, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances for producing a medicament for mGluR5 receptor regulation, preferably for inhibiting the mGluR5 receptor.

It is preferred to use at least one substituted propiolic acid amide according to the invention of the above-stated general formula I, including above-excepted compounds, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically compatible auxiliary substances for producing a medicament for the prevention and/or treatment of disorders and/or diseases which are mediated at least in part by mGluR5 receptors.

Particular preference is given to the use of at least one substituted propiolic acid amide according to the invention of the above-stated general formula I including above-excepted compounds, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances for producing a medicament for the prevention and/or treatment of pain, preferably of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's chorea; cognitive diseases, preferably cognitive deficiency states, particularly preferably attention deficit syndrome (ADS); anxiety states; panic attacks; epilepsy; coughing; urinary incontinence; diarrhoea; pruritus; schizophrenia; cerebral ischaemic episodes; muscle spasms; cramps; pulmonary diseases, preferably selected from the group comprising asthma and pseudocroup; regurgitation (vomiting); stroke; dyskinesia; retinopathy; lack of drive; laryngitis); disorders of food intake, preferably selected from the group consisting of bulimia, cachexia, anorexia and obesity; dependency on alcohol; dependency on medicines; dependency on drugs, preferably nicotine- and/or cocaine-dependency; alcohol abuse; abuse of medicines; drug abuse; preferably nicotine and/or cocaine abuse; withdrawal symptoms associated with dependency on alcohol, medicines and/or drugs (in particular nicotine- and/or cocaine-dependency); development of tolerance towards medicines, in particular towards natural or synthetic opioids; gastro-oesophageal reflux syndrome; gastro-oesophageal reflux disease; irritable bowel syndrome; for diuresis; for antinatriuresis; for influencing the cardiovascular system; for increasing vigilance; for increasing libido; for modulating locomotor activity or for local anaesthesia.

Very particular preference is given to the use of at least one substituted propiolic acid amide according to the invention of the above-stated general formula I including above-excepted compounds, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances for producing a medicament for the prevention and/or treatment of pain, preferably of pain selected from the group consisting of acute pain, chronic pain, neuropathic pain and visceral pain; anxiety states; panic attacks; dependency on alcohol; dependency on medicines; disorders of food intake, preferably selected from the group consisting of bulimia, cachexia, anorexia and obesity; dependency on drugs, preferably nicotine- and/or cocaine-dependency; alcohol abuse; abuse of medicines; drug abuse; preferably nicotine and/or cocaine abuse; withdrawal symptoms associated with dependency on alcohol, medicines and/or drugs (in particular nicotine- and/or cocaine-dependency); development of tolerance towards medicines and/or drugs, preferably towards natural or synthetic opioids; gastro-oesophageal reflux syndrome, gastro-oesophageal reflux disease and irritable bowel syndrome.

Still greater preference is given to the use of at least one substituted propiolic acid amide according to the invention of the above-stated general formula I including above-excepted compounds, in each case optionally in the form of one of the pure stereoisomers thereof, in particular enantiomers or diastereomers, the racemates thereof or in the form of a mixture of stereoisomers, in particular the enantiomers and/or diastereomers, in any desired mixing ratio, or in each case in the form of a corresponding salt, or in each case in the form of a corresponding solvate, and optionally one or more pharmaceutically acceptable auxiliary substances for producing a medicament for the prevention and/or treatment of pain, preferably of pain selected from the group consisting of acute pain, chronic pain, neuropathic pain and visceral pain, anxiety states and panic attacks.

The medicament according to the invention is suitable for administration to adults and children including small children and babies.

The medicament according to the invention may be formulated as a liquid, semisolid or solid dosage form, for example in the form of solutions for injection, drops, succi, syrups, sprays, suspensions, tablets, patches, capsules, dressings, suppositories, ointments, creams, lotions, gels, emulsions, aerosols or in multiparticulate form, for example in the form of pellets or granules, optionally pressed into tablets, packaged in capsules or suspended in a liquid, and may also be administered as such.

In addition to at least one substituted propiolic acid amide according to the invention of the above-stated general formula I, optionally in the form of one of the pure stereoisomers thereof, in particular enantiomers or diastereomers, the racemate thereof or in the form of mixtures of the stereoisomers, in particular the enantiomers or diastereomers, in any desired mixing ratio, or optionally in the form of a corresponding salt or in each case in the form of a corresponding solvate, the medicament according to the invention conventionally contains further physiologically acceptable pharmaceutical auxiliary substances, which may preferably be selected from the group consisting of matrix materials, fillers, solvents, diluents, surface-active substances, dyes, preservatives, disintegrants, slip agents, lubricants, aromas and binders.

Selection of the physiologically acceptable auxiliary substances and the quantities thereof which are to be used depends upon whether the medicament is to be administered orally, subcutaneously, parenterally, intravenously, intraperitoneally, intradermally, intramuscularly, intranasally, buccally, rectally or topically, for example onto infections of the skin, mucous membranes and eyes. Preparations in the form of tablets, coated tablets, capsules, granules, pellets, drops, succi and syrups are preferred for oral administration, while solutions, suspensions, readily reconstitutible dried preparations and sprays are preferred for parenteral, topical and inhalatory administration.

The substituted propiolic acid amides of the above-stated general formula I used in the medicament according to the invention in a depot in dissolved form or in a dressing, optionally with the addition of skin penetration promoters, are suitable percutaneous administration preparations.

Orally or percutaneously administrable preparation forms may release the respective substituted propiolic acid amides of the above-stated general formula I in delayed manner.

Production of the pharmaceutical preparations according to the invention proceeds with the assistance of conventional means, devices, methods and processes well known to a person skilled in the art, such as are described for example in “Remington's Pharmaceutical Sciences”, ed. A. R. Gennaro, 17th ed., Mack Publishing Company, Easton, Pa., 1985, in particular in part 8, chapters 76 to 93. The corresponding description is hereby introduced as a reference and is deemed to be part of the disclosure.

The quantity of the particular substituted propiolic acid amide of the above-stated general formula I to be administered to the patient may vary and is for example dependent on the weight or age of the patient and on the mode of administration, the indication and the severity of the disease. Conventionally, at least one such compound is administered in a quantity of 0.05 to 100 mg/kg, preferably of 0.05 to 10 mg/kg, of patient body weight.

### Pharmacological Methods:

I. Method for Determining the Inhibition of [³H]-MPEP Binding in the mGluR5 Receptor Binding Assay

Pig brain homogenate is produced by homogenisation (Polytron Pt 3000, Kinematica AG, 10,000 revolutions per minute for 90 seconds) of pig brain hemispheres without medulla, cerebellum and pons in a buffer of pH 8.0 (30 mM Hepes, Sigma, order number H3375+1 tablet Complete Roche Diagnostics, order number 1836145 made up to 100 ml) in a ratio of 1:20 (brain weight/volume) and differential centrifugation at 900×g and 40,000×g. 450 μg of protein from brain homogenate is incubated in each case in 250 μl incubation batches in 96-well microtitre plates with 5 nM ³[H]-MPEP (Tocris, order number R1212) (MPEP=2-methyl-6-(3-methoxyphenyl)-ethynylpyridine) and the compounds to be investigated (10 μM in the test) in the buffer (as above) at room temperature for 60 min.

The batches are then filtered with the assistance of a Brandel Cell Harvester (Brandel, Robotic 9600 model) on Unifilter plates with glass filter mats (Perkin Elmer, order number 6005177) and then washed 3 times with buffer (as above) using 250 μl per sample. The filter plates are then dried for 60 min at 55° C. Then 30 μl of Ultima Gold™ scintillating material (Packard BioScience, order number 6013159) is added per well and after 3 hours the samples are measured using the β counter (Microbeta, Perkin Elmer). Nonspecific binding is determined by the addition of 10 μM MPEP (Tocris, order number 1212).

II. Method for Determining Ca²⁺ Influx in the mGluR5 Receptor Assay

An agonistic and/or antagonistic substance action on the mGluR5 receptor of the rat species may be determined with the following assay. According to this assay, intracellular Ca²⁺ release is quantified after activation of the mGluR5 receptor with the assistance of a Ca²⁺-sensitive dye (type Fluo-4, Molecular Probes Europe BV, Leiden, Netherlands) in a FlexStation (Molecular Devices, Sunnyvale, USA).

### Preparation of Cortical Neurones:

Cortical neurones are dissected out from postnatal rats (P2-6) under sterile conditions. To this end, the cortex is removed and transferred directly into collagenase solution (PAA Laboratories GmbH, Cölbe, Germany) and incubated for 45 minutes in a heated agitator (37° C., 300 revolutions per minute). Then the collagenase solution is removed and the tissue combined with culture medium.

### Culture Medium (100 ml):

Neurobasal medium (Gibco Invitrogen GmbH, Karlsruhe, Germany)

### 2 mM L-glutamine (Sigma, Taufkirchen, Germany)

1 vol. % of antibiotic/antimycotic solution (PAA Laboratories GmbH, Cölbe, Germany)
15 ng/ml NGF (Gibco Invitrogen GmbH, Karlsruhe, Germany)
1 ml B27 supplement (Gibco Invitrogen GmbH, Karlsruhe, Germany)
1 ml ITS supplement (Sigma, Taufkirchen, Germany)

The cells are isolated by resuspension and centrifuged through a 70 μm filter insert (BD Biosciences, Heidelberg, Germany) after addition of 15 ml of neurobasal medium. The resultant cell pellet is resuspended in culture medium. Then, the cells are plated out onto poly-D-lysine-coated, black 96-well plates with a clear base (BD Biosciences, Heidelberg, Germany), which have previously been additionally coated with laminin (2 μg/cm², Gibco Invitrogen GmbH, Karlsruhe, Germany). Cell density amounts to 15,000 cells/well. The cells are incubated at 37° C. and 5% CO₂ and the medium is changed on the 2nd or 3rd day after dissection. Depending on cell growth, the functional investigation may be performed on the 3rd-7th day after dissection.

### Description of the Functional Ca²⁺ Influx Assay

20,000 CHO-hmGluR5 cells/well (Euroscreen, Gosselies, Belgium) are pipetted out into 96 well plates (BD Biosciences, Heidelberg, Germany, Ref. 356640, clear bottom, 96 well, poly-D-lysine) and incubated overnight in HBSS buffer (Gibco No. 14025-050) with the following additives: 10% FCS (GIBCO, 10270-106) and doxycycline (BD Biosciences Clontech 631311 600 ng/ml).

For the purpose of functional investigation, the cells were loaded with 2 μM Fluo-4 and 0.01 vol. % Pluronic F127 (Molecular Probes Europe BV, Leiden, Netherlands) in HBSS buffer (Hank's buffered saline solution, Gibco Invitrogen GmbH, Karlsruhe, Germany) with Probenicid (Sigma P8761, 0.69 mg/ml) for 30 min at 37° C.

The cells are then washed 3 times with washing buffer (HBSS buffer, Gibco No. 14025-050, with Probenicid (Sigma P8761, 0.69 [mg]/ml) and then resuspended with the same buffer to make up to 100 μl. After 15 min the plates are transferred for determination of Ca²⁺ measurements in the presence of DHPG ((S)-3,5-dihydroxyphenylglycine, Tocris Biotrend Chemikalien GmbH, Cologne, Germany, final DHPG concentration: 10 μM) and in the presence or absence of test substances into a FLuorometric Imaging Plate Reader (FLIPR, Molecular Devices, Sunnyvale, Calif.).

Ca²⁺-dependent fluorescence is measured before and after addition of test substances. Quantification proceeds by measuring the highest fluorescence intensity over time.

After recording a fluorescence baseline for 10 s, 50 μl of test substance solution (various test substance concentrations in HBSS buffer with 1% DMSO and 0.02% Tween 20, Sigma) are added and the fluorescence signal is measured for 6 min. Then, 50 μl of DHPG solution ((S)-3,5-dihydroxyphenylglycine, Tocris Biotrend Chemikalien GmbH, Cologne, Germany, final DHPG concentration: 10 μM) are added and Ca²⁺ influx is simultaneously measured for 60 s. The final DMSO concentration amounts to 0.25% and the final Tween 20 content amounts to 0.005%. The data are analysed with Microsoft Excel and GraphPad Prism. The dose-response curves are calculated with non-linear regression and IC₅₀ values are determined. Each data point is determined in triplicate and IC₅₀ values are averaged from a minimum of 2 independent measurements.

Ki values are calculated using the following formula: Ki=IC50/(1+(AG_{conc}/EC50)).

AG_{conc}=10 μM; EC50 corresponds to the DHPG concentration which is necessary for the semimaximal influx of Ca²⁺.

### III. Formaldehyde Test in Rats:

The formaldehyde test (Dubuisson, D. and Dennis, S. G., 1977, Pain, 4, 161-174) is a model of acute and chronic pain. A biphasic nociceptive response is induced in freely mobile test animals by a single formaldehyde injection into the dorsal side of a hind paw, said response being detected by the observation of three clearly distinguishable behaviour patterns. The response is in two phases: phase 1=immediate response (duration up to 10 min; paw shaking, licking), phase 2=late response (after a resting phase; likewise paw shaking, licking; duration up to 60 min). The 1st phase reflects direct stimulation of the peripheral nocisensors with an elevated spinal nociceptive input or glutamate release (acute pain phase); the 2nd phase reflects spinal and peripheral hypersensitisation (chronic pain phase). In the investigations presented here, it was the chronic pain component (phase 2) which was evaluated.

Formaldehyde is administered subcutaneously in a volume of 50 μl and a concentration of 5% into the dorsal side of the right hind paw of each animal. The substances to be tested are administered orally (per os), intravenously (i.v.) or intraperitoneally (i.p.) 30 min before the formaldehyde injection. The specific behavioural changes, such as raising and shaking the paw, changes in weight bearing of the animal and biting and licking responses are observed and recorded over the observation period of 21 to 27 min after the formaldehyde injection. The various behaviours are summarised as a “pain rate” (PR), which is the calculated nociceptive response averaged over 3 min sub-intervals. The PR is calculated on the basis of a numerical weighting (=in each case a factor of 1, 2, 3) of the observed behaviours (corresponding behaviour score 1, 2, 3) and is calculated using the following formula:

PR=[(T₀×0)+(T₁×1)+(T₂×2)+(T₃×3)]/180

wherein T₀, T₁, T₂, and T₃ correspond to the time in seconds for which the animal exhibited behaviour 0, 1, 2 or 3 respectively. Group size is 10 animals (n=10).

The following Examples serve to explain the invention in more detail, but do not restrict the general concept of the invention.

### EXAMPLES

The yields of the compounds produced are not optimised.

All temperatures are uncorrected.

The chemicals and solvents used were purchased from conventional suppliers (Acros, Avocado, Aldrich, Bachem, Fluka, Lancaster, Maybridge, Merck, Sigma, TCI, etc.) or synthesised.

Silica gel 60 (0.040-0.063 mm) from E. Merck, Darmstadt, was used as the stationary phase for the column chromatography.

Thin-layer chromatography was performed with pre-coated silica gel 60 F 254 HPTLC plates from E. Merck, Darmstadt.

The mixture ratios for solvents, mobile solvents or for chromatographic investigations are always stated in volume/volume.

Analysis was performed by mass spectroscopy and NMR.

### Abbreviations:

aq. aqueous
Brine saturated aqueous NaCl solution
CDI 1,1′-carbonyldiimidazole
DCE 1,2-dichloroethane
DCM dichloromethane
DIC N,N′-diisopropylcarbodiimide

DIPEA diisopropylethylamine

### DMF N,N-dimethylformamide

EA ethyl acetate
EtOH ethanol
sat. saturated
HOBT 1-hydroxy-1H-benzotriazole
MeCN acetonitrile
MTBE tert.-butyl methyl ether
NEt₃ triethylamine
Pd/C palladium 10%, on activated carbon
RT room temperature
CC column chromatography
TBTU O-(benzotriazol-1-yl)-N,N,N′,N′-tetramethyluronium tetrafluoroborate
THF tetrahydrofuran

### Example 5

### Synthesis of 2-(3-(3-chlorophenyl)-propiolyl)-1,2,3,4-tetrahydro-isoquinoline

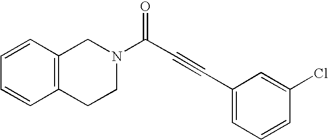

361 mg (2.0 mmol) of 3-chlorophenyl-propiolic acid, 349 μl (2.0 mmol) of DIPEA and 649 mg (2.0 mmol) of TBTU were added to a solution of 250 μl (2.0 mmol) of 1,2,3,4-tetrahydroisoquinolinyl in MeCN (15 ml). Then the reaction solution was stirred for 2 h at RT and the solvent removed under a vacuum. The residue was resuspended in DCM and washed successively with water and brine and dried over MgSO₄. After filtration and removal of the solvent under a vacuum, CC (DCM/EtOH 10:1) was performed with the residue, 220 mg (0.74 mmol, 37%) of 2-(3-(3-chlorophenyl)-propiolyl)-1,2,3,4-tetrahydro-isoquinoline being obtained. MS [MH+] 296.1

### Example 7

### 2-(3-pyrid-2-yl-propiolyl)-1,2,3,4-tetrahydro-isoquinoline

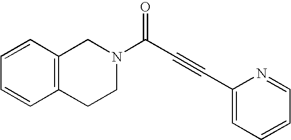

### a) Synthesis of 2-propiolyl-1,2,3,4-tetrahydro-isoquinoline

616 μl (10.0 mmol) of propiolic acid, 1.74 ml (10.0 mmol) of DIPEA and 3.21 g (10.0 mmol) of TBTU were added at −10° C. to a solution of 1.25 ml (10.0 mmol) of 1,2,3,4-tetrahydro-isoquinoline in THF (50 ml). After heating to RT, the reaction solution was stirred for 3 h at this temperature. The resultant precipitate was filtered out and the solvent was removed under a vacuum. The residue obtained was resuspended in DCM and washed successively with water and brine and dried over MgSO₄. After filtration and removal of the solvent under a vacuum, CC (DCE/EtOH 20:1) was performed with the residue, 480 mg (2.6 mmol, 26%) of 2-propiolyl-1,2,3,4-tetrahydro-isoquinoline being obtained.

### b) Synthesis of 2-(3-pyrid-2-yl-propiolyl)-1,2,3,4-tetrahydro-isoquinoline

34 mg (0.048 mmol) of Pd(PPh₃)₂Cl₂, 18 mg (0.095 mmol) of CuI and 728 μl (5.2 mmol) of NEt₃ were added to a solution of 480 mg (2.6 mmol) of 2-propiolyl-1,2,3,4-tetrahydro-isoquinoline and 276 μl (2.6 mmol) of 2-iodopyridine in DMF (20 ml) and then the reaction solution was stirred for 1 h at 70° C. Then the reaction solution was poured onto water. The resultant mixture was extracted with DCM and the organic phase was dried over MgSO₄. After filtration and removal of the solvent under a vacuum, CC (EA/hexane 1:1) was performed with the residue, 256 mg (0.98 mmol, 38%) of 2-(3-pyrid-2-yl-propiolyl)-1,2,3,4-tetrahydro-isoquinoline being obtained. MS [MH+] 263.1

### Example 8

### Synthesis of 3-methyl-5-(3-phenyl-propiolyl)-4,5,6,7-tetrahydro-isoxazolo[4,5-c]pyridine

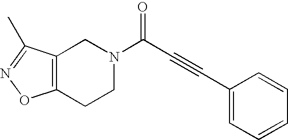

243 mg (0.78 mmol) of phenylpropiolic acid pentafluorophenyl ester and 166 μL (0.98 mmol) of DIPEA were added to a solution of 90 mg (0.65 mmol) of 3-methyl-4,5,6,7-tetrahydro-isoxazolo[4,5-c]pyridine in THF/DMF [10:1 (10 ml)] and the reaction solution was then stirred for 15 h at RT. The mixture was evaporated under a vacuum and, after purification by column chromatography (EE/hexane 1:1), 110 mg (0.41 mmol, 64%) of 3-methyl-5-(3-phenyl-propiolyl)-4,5,6,7-tetrahydro-isoxazolo[4,5-c]pyridine were obtained. MS [MH+] 267.1

### Example 10

### Synthesis of 7-(3-phenyl-propiolyl)-5,6,7,8-tetrahydro-[1,2,4]triazolo[4,3-a]pyrazine

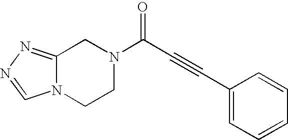

0.5 g (3.4 mmol) of phenylpropiolic acid were combined with a solution of 0.55 g (3.4 mmol) of 5,6,7,8-tetrahydro-[1,2,4]triazolo[4,3-a]pyrazine hydrochloride in DMF (3 ml). Then 533 μl (3.4 mmol) of DIC, 581 μl (7.6 mmol) of DIPEA and 462 mg (3.4 mmol) of HOBT were added and the reaction mixture was stirred for 12 h at RT. Addition of 1 M aq. Na₂CO₃ solution and hexane led to the formation of a brown solid, which was washed after filtration in each case three times with hexane, MTBE, EE and DCM. The EA and DCM phases were washed in each case with brine, then combined and dried over MgSO₄. After filtration and removal of the solvent under a vacuum, the residue was purified by column chromatography (EA/MeOH 4:1), 335 mg (1.33 mmol, 39%) of 7-(3-phenyl-propiolyl)-5,6,7,8-tetrahydro-[1,2,4]triazolo[4,3-a]pyrazine being obtained. MS [MH+] 253.1

### Examples 13, 15 and 16

### Synthesis of 2-(3-(3-chlorophenyl)-propiolyl)-7,6-dihydroxy-1,2,3,4-tetrahydro-isoquinoline, 2-(3-(3-chlorophenyl)-propiolyl)-7-hydroxy-6-methoxy-1,2,3,4-tetrahydro-isoquinoline and 2-(3-(3-chlorophenyl)-propiolyl)-6-hydroxy-7-methoxy-1,2,3,4-tetrahydro-isoquinoline

To a solution cooled to −78° C. of 712 mg (2.0 mmol) of 2-(3-(3-chlorophenyl)-propiolyl)-6,7-dimethoxy-1,2,3,4-tetrahydro-isoquinoline (Example 11) in DCM (50 ml), there was added dropwise at this temperature a solution of 578 μl (6.0 mmol) boron tribromide in DCM (10 ml). Then the mixture was heated to RT over a period of 16 h. The reaction solution was poured with cooling and stirring into a 20% aq. Na₂CO₃ soln. The phases were separated and the organic phase was dried over MgSO₄, filtered and evaporated under a vacuum. From the residue it was possible to obtain by CC (SiO₂, chloroform) 218 mg (0.64 mmol, 32%) of a mixture of 2-(3-(3-chlorophenyl)-propiolyl)-7-hydroxy-6-methoxy-1,2,3,4-tetrahydro-isoquinoline and 2-(3-(3-chlorophenyl)-propiolyl)-6-hydroxy-7-methoxy-1,2,3,4-tetrahydro-isoquinoline, and 120 mg (0.37 mmol, 18%) of 2-(3-(3-chlorophenyl)-propiolyl)-7,6-dihydroxy-1,2,3,4-tetrahydro-isoquinoline.

Example 13: MS [MH+] 328.1

Examples 15 and 16: MS [MH+] 342.1

### Example 20

### 2-(3-(3-chlorophenyl)-propiolyl)-6-methyl-1,2,3,4-tetrahydro-isoquinoline

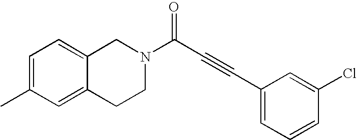

### a) Synthesis of 6-methyl-1,2,3,4-tetrahydro-isoquinoline

100 mg of Pd/C were added to a solution of 1.06 g (7.4 mmol) of 6-methyl-isoquinoline in MeOH (30 ml). Hydrogenation was then performed for 6 h with hydrogen at a pressure of 9 bar. Thereafter, filtering was performed through diatomaceous earth and the filtrate was evaporated under a vacuum. CC (SiO₂, chloroform) was carried out with the residue, 500 mg (3.4 mmol, 46%) of 6-methyl-1,2,3,4-tetrahydro-isoquinoline being obtained.

### b) Synthesis of 2-(3-(3-chlorophenyl)-propiolyl)-6-methyl-1,2,3,4-tetrahydro-isoquinoline

1.88 g (≈2.44 mmol) of PS-carbodiimide resin were added to a solution of 120 mg (0.82 mmol) of 6-methyl-1,2,3,4-tetrahydro-isoquinoline and 221 mg (1.22 mmol) of 3-(3-chlorophenyl)propiolic acid in DCM (10 ml) and the reaction mixture was shaken for 3 h at RT. Then the resin was filtered out and washed with DCM and MeOH. The filtrate was evaporated under a vacuum and CC (SiO₂, DCE) was performed with the residue, 148 mg (0.47 mmol, 58%) of 2-(3-(3-chlorophenyl)-propiolyl)-6-methyl-1,2,3,4-tetrahydro-isoquinoline being obtained.

MS [MH+] 310.1

### Example 21

### 2-(3-(3-chlorophenyl)-propiolyl)-2,3,4,5-tetrahydro-1H-benzo[c]azepine

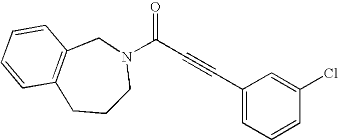

### a) Synthesis of 2,3,4,5-tetrahydrobenzo[c]azepin-1-one

2.72 g (41.8 mmol) of sodium azide were added in portions to a solution of 6.11 g (41.8 mmol) of 3,4-dihydronaphthalen-1(2H)-one in conc. hydrochloric acid (75 ml) at 0° C. over a period of 1 h. Then stirring was performed for another 30 min at 0° C. and thereafter heating to RT over a period of 16 h. The reaction solution was poured into ice water and made basic (pH 9) with potassium carbonate. Then extraction was carried out with DCM. The organic phase was dried over MgSO₄, filtered and evaporated under a vacuum. The residue was crystallised from hexane. CC (SiO₂, hexane/EA 1:2) was performed with the resultant residue, 1.8 g (11.1 mmol, 27%) of 2,3,4,5-tetrahydrobenzo[c]azepin-1-one being obtained.

### b) Synthesis of 2,3,4,5-tetrahydro-1H-benzo[c]azepine

A solution of 1.48 g (39.0 mmol) of lithium aluminium hydride in THF (50 ml) was added dropwise to a solution cooled to 0° C. of 1.8 g (11.1 mmol) of 2,3,4,5-tetrahydro-benzo[c]azepin-1-one in THF (20 ml), the temperature not rising above 5° C. The mixture was then refluxed for 4 h. After cooling to RT, the reaction solution was poured into a 50% aq. KOH-soln. Insoluble precipitates were filtered out and the filtrate was dried over MgSO₄, filtered and evaporated under a vacuum. 545 mg (3.7 mmol, 33%) of 2,3,4,5-tetrahydro-1H-benzo[c]azepine were obtained, which were reacted without further purification in the next stage.

### c) Synthesis of 2-(3-(3-chlorophenyl)-propiolyl)-2,3,4,5-tetrahydro-1H-benzo-[c]-azepine

4.6 g (≈6.0 mmol) of PS-carbodiimide resin were added to a solution of 294 mg (2.0 mmol) of 2,3,4,5-tetrahydro-1H-benzo[c]azepine and 542 mg (3.0 mmol) of 3-(3-chlorophenyl)propiolic acid in DCM (27 ml) and the reaction mixture was shaken for 16 h at RT. Then the resin was filtered out and washed with DCM and MeOH. The filtrate was evaporated under a vacuum and CC (SiO₂, hexane/EA 4:1) was performed with the residue, 343 mg (1.1 mmol, 55%) of 2-(3-(3-chlorophenyl)-propiolyl)-2,3,4,5-tetrahydro-1H-benzo-[c]-azepine being obtained.

MS [MH+] 310.1

### Example 28

### Synthesis of 2-(3-(3-chlorophenyl)-propiolyl)-7-hydroxy-1,2,3,4-tetrahydro-quinoline

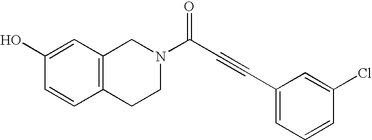

178 mg (1.1 mmol) of CDI were added to a solution of 181 mg (1.0 mmol) of 3-chlorophenyl-propiolic acid in DCE (10 ml). Then the reaction solution was stirred for 1 h at RT. Then a suspension of 209 mg (1.0 mmol) 1,2,3,4-tetrahydro-isoquinolin-7-ol acetate and 700 μl (4.0 mmol) of DIPEA in DCE (10 ml) was added and stirring was performed for a further 16 h at RT. The reaction solution was diluted with DCE (30 ml) and washed with brine (10 ml). The organic phase was dried over MgSO₄. After filtration and removal of the solvent under a vacuum, CC (hexane/EtOAc 3:2) was performed with the residue, 75 mg (0.24 mmol, 24%) of 2-(3-(3-chlorophenyl)-propiolyl)-7-hydroxy-1,2,3,4-tetrahydro-quinoline being obtained.

Examples 1, 2, 3, 4, 6, 11 and 12 were produced using the method described in Example 5. Examples 9, 14, 17, 18, 19 and 22 were produced using the method described in Example 10. Examples 23, 24, 25, 26, 27, 29, 30 and 31 were produced using the method described in Example 21 Section c). Table 1 summarises the results.

The starting compounds necessary in each case for this purpose are known to a person skilled in the art.

TABLE 1

| Ex. | Name |
|---|---|
| 1 | 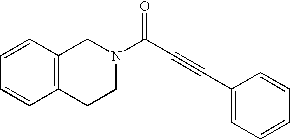 |
| | 2-(3-phenyl-propiolyl)-1,2,3,4-tetrahydro-isoquinoline |
| | MS [MH+] 261.1 |
| 2 | 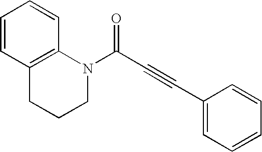 |
| | 1-(3-phenyl-propiolyl)-1,2,3,4-tetrahydro-quinoline |
| | MS [MH+] 262.1 |
| 3 | 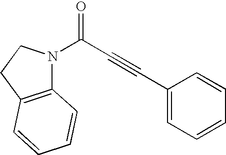 |
| | 1-(3-phenyl-propiolyl)-2,3-dihydro-indole |
| | MS [MH+] 248.1 |
| 4 | 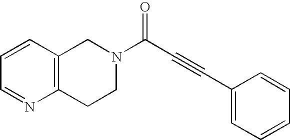 |
| | 6-(3-phenyl-propiolyl)-5,6,7,8-tetrahydro-[1,6]naphthyridine |
| | MS [MH+] 263.1 |
| 6 | 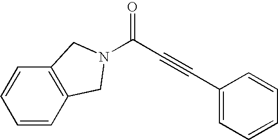 |
| | 2-(3-phenyl-propiolyl)-2,3-dihydro-isoindole |
| | MS [MH+] 248.1 |
| 9 | 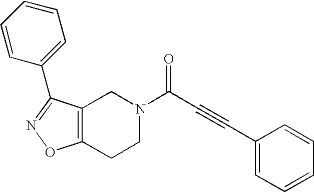 |
| | 3-phenyl-5-(3-phenyl-propiolyl)-4,5,6,7-tetrahydro-isoxazolo[4,5- |
| | c]pyridine MS [MH+] 329.1 |
| 11 | 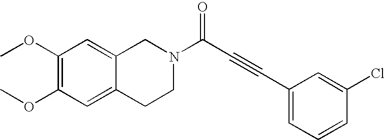 |
| | 2-(3-(3-chlorophenyl)-propiolyl)-6,7-dimethoxy-1,2,3,4-tetrahydro- |
| | isoquinoline MS [MH+] 356.1 |
| 12 | 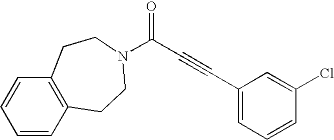 |
| | 3-(3-(3-chlorophenyl)-propiolyl)-1,2,4,5-tetrahydrobenzo[d]azepine |
| | MS [MH+] 310.1 |
| 14 | 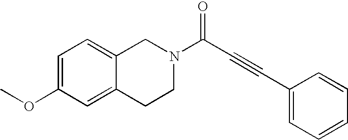 |
| | 6-methoxy-2-(3-phenyl-propiolyl)-1,2,3,4-tetrahydro-isoquinoline |
| | MS [MH+] 292.1 |
| 17 | 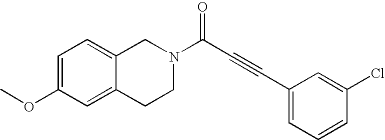 |
| | 2-(3-(3-chlorophenyl)-propiolyl)-6-methoxy-1,2,3,4-tetrahydro- |
| | isoquinoline MS [MH+] 326.1 |
| 18 | 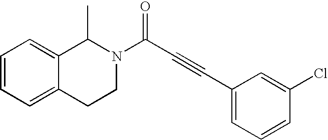 |
| | 2-(3-(3-chlorophenyl)-propiolyl)-1-methyl-1,2,3,4-tetrahydro- |
| | isoquinoline MS [MH+] 310.1 |
| 19 | 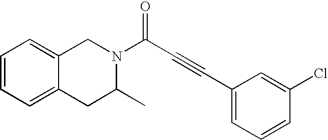 |
| | 2-(3-(3-chlorophenyl)-propiolyl)-3-methyl-1,2,3,4-tetrahydro- |
| | isoquinoline MS [MH+] 310.1 |
| 22 | 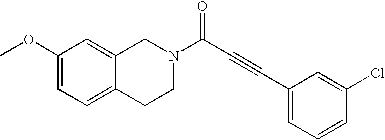 |
| | 2-(3-(3-chlorophenyl)-propiolyl)-7-methoxy-1,2,3,4-tetrahydro- |
| | isoquinoline MS [MH+] 326.2 |
| 23 | 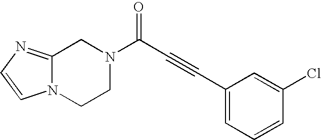 |
| | 7-(3-(3-chlorophenyl)-propiolyl)-5,6,7,8-tetrahydroimidazo[1,2- |
| | a]pyrazine MS [MH+] 286.1 |
| 24 | 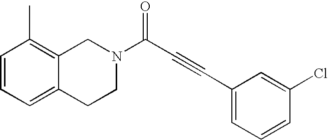 |
| | 2-(3-(3-chlorophenyl)-propiolyl)-8-methyl-1,2,3,4-tetrahydro- |
| | isoquinoline MS [MH+] 310.1 |
| 25 | 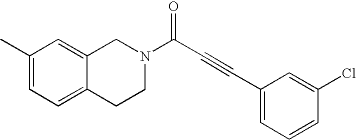 |
| | 2-(3-(3-chlorophenyl)-propiolyl)-7-methyl-1,2,3,4-tetrahydro- |
| | isoquinoline MS [MH+] 310.1 |
| 26 | 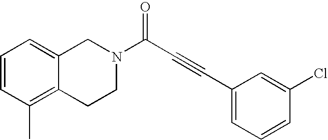 |
| | 2-(3-(3-chlorophenyl)-propiolyl)-5-methyl-1,2,3,4-tetrahydro- |
| | isoquinoline MS [MH+] 310.1 |
| 27 | 2-(3-(3-chlorophenyl)-propiolyl)-1,2,3,4-tetrahydropyrrolo[1,2- |
| | a]pyrazine MS [MH+] 285.1 |
| 29 | 2-(3-(3-chlorophenyl)-propiolyl)-8-methoxy-1,2,3,4-tetrahydro- |
| | isoquinoline MS [MH+] 326.1 |
| 30 | 2-(3-(3-chlorophenyl)-propiolyl)-5-methoxy-1,2,3,4-tetrahydro- |
| | isoquinoline MS [MH+] 326.1 |
| 31 | 2-(3-(3-chlorophenyl)-propiolyl)-1,8-dimethyl-1,2,3,4-tetrahydro- |
| | isoquinoline MS [MH+] 324.1 |

General Synthesis Method for the Reaction of Secondary Amines of the General Formula II with Aromatically Substituted Propiolic Acids of the General Formula R¹—C≡C—C(═O)—OH

A solution of CDl (105 μmol, 1.05 equivalents) in DCM (1.05 ml) was added to a solution of the respective aromatically substituted propiolic acid of the general formula R¹—C≡C—C(═O)—OH (100 μmol) in DCM (2 ml). The reaction solution was stirred for 1 h at 20° C. and then combined with a solution of the respective secondary amine of the general formula II (100 μmol, 1.0 equivalents) in DCM (1 ml). Thereafter stirring was performed for a further 16 h at RT. The reaction mixture was then combined with water (3 ml) and the phases were separated. The organic phase was washed with water (3 ml) and with brine (3 ml), dried over MgSO₄ and filtered. After removal of the solvent under a vacuum the respective target compound was isolated from the residue by means of preparative HPLC.

Synthesis of Examples 32 to 79 (Table 2) was carried out using the described general method for reacting secondary amines with aromatically substituted propiolic acids. Analysis was carried out by HPLC-MS, purity in all cases being >85%. It is apparent to a person skilled in the art which starting compounds and intermediates were used in each case.

TABLE 2

| Ex. | Name | Mass [MH+] |
|---|---|---|
| 32 | 2-(3-(2,4-difluorophenyl)-propiolyl)-1,2,3,4-tetrahydro-isoquinoline | 298.1 |
| 33 | 1-(3-(2,4-difluorophenyl)-propiolyl)-2-methyl-indoline | 298.1 |
| 34 | 2-(3-(3-methoxyphenyl)-propiolyl)-1,2,3,4-tetrahydro-isoquinoline | 292.1 |
| 35 | 2-(3-(4-fluoro-3-methylphenyl)-propiolyl)-1,2,3,4-tetrahydro-isoquinoline | 294.1 |
| 36 | 1-(3-(4-fluoro-3-methylphenyl)-propiolyl)-2-methyl-indoline | 294.1 |
| 37 | 2-(3-(2-fluorophenyl)-propiolyl)-1,2,3,4-tetrahydro-isoquinoline | 280.1 |
| 38 | 1-(3-(2-fluorophenyl)-propiolyl)-2-methyl-indoline | 280.1 |
| 39 | 2-(3-(4-tolyl)-propiolyl)-1,2,3,4-tetrahydro-isoquinoline | 276.1 |
| 40 | 1-(3-(4-tolyl)-propiolyl)-2-methyl-indoline | 276.1 |
| 41 | 2-(3-(4-trifluoromethylphenyl)-propiolyl)-1,2,3,4-tetrahydro-isoquinoline | 330.1 |
| 42 | 1-(3-(4-trifluoromethylphenyl)-propiolyl)-2-methyl-indoline | 330.1 |
| 43 | 2-(3-phenyl-propiolyl)-1,2,3,4-tetrahydro-isoquinoline | 262.1 |
| 44 | 1-(3-phenyl-propiolyl)-2-methyl-indoline | 262.1 |
| 45 | 2-(3-(2,3-dimethylphenyl)-propiolyl)-1,2,3,4-tetrahydro-isoquinoline | 290.1 |
| 46 | 1-(3-(2,3-dimethylphenyl)-propiolyl)-2-methyl-indoline | 290.1 |
| 47 | 2-(3-(3,5-dimethylphenyl)-propiolyl)-1,2,3,4-tetrahydro-isoquinoline | 290.1 |
| 48 | 1-(3-(3,5-dimethylphenyl)-propiolyl)-2-methyl-indoline | 290.1 |
| 49 | 2-(3-(2,4-dichlorophenyl)-propiolyl)-1,2,3,4-tetrahydro-isoquinoline | 330.0 |
| 50 | 1-(3-(2,4-dichlorophenyl)-propiolyl)-2-methyl-indoline | 330.0 |
| 51 | 2-(3-(3-tolyl)-propiolyl)-1,2,3,4-tetrahydro-isoquinoline | 276.1 |
| 52 | 1-(3-(3-tolyl)-propiolyl)-2-methyl-indoline | 276.1 |
| 53 | 2-(3-(4-methoxyphenyl)-propiolyl)-1,2,3,4-tetrahydro-isoquinoline | 292.1 |
| 54 | 1-(3-(4-methoxyphenyl)-propiolyl)-2-methyl-indoline | 292.1 |
| 55 | 2-(3-(2-thienyl)-propiolyl)-1,2,3,4-tetrahydro-isoquinoline | 268.1 |
| 56 | 1-(3-(2-thienyl)-propiolyl)-2-methyl-indoline | 268.1 |
| 57 | 1-(3-(4-tolyl)-propiolyl)-1,2,3,4-tetrahydro-quinoline | 276.1 |
| 58 | 1-(3-(4-trifluoromethylphenyl)-propiolyl)-1,2,3,4-tetrahydro-quinoline | 330.1 |
| 59 | 1-(3-(2,3-dimethylphenyl)-propiolyl)-1,2,3,4-tetrahydro-quinoline | 290.1 |
| 60 | 1-(3-(2,4-dichlorophenyl)-propiolyl)-1,2,3,4-tetrahydro-quinoline | 330.0 |
| 61 | 1-(3-(2,4-dimethylphenyl)-propiolyl)-1,2,3,4-tetrahydro-quinoline | 290.1 |
| 62 | 1-(3-(4-tert.-butylphenyl)-propiolyl)-1,2,3,4-tetrahydro-quinoline | 318.2 |
| 63 | 1-(3-(2,4-dimethylphenyl)-propiolyl)-2-methyl-indoline | 290.1 |
| 64 | 2-(3-(4-fluorophenyl)-propiolyl)-1,2,3,4-tetrahydro-isoquinoline | 280.1 |
| 65 | 1-(3-(4-fluorophenyl)-propiolyl)-2-methyl-indoline | 280.1 |
| 66 | 1-(3-(3-chlorophenyl)-propiolyl)-2-methyl-indoline | 296.1 |
| 67 | 2-(3-(3-fluoro-4-methylphenyl)-propiolyl)-1,2,3,4-tetrahydro-isoquinoline | 294.1 |
| 68 | 1-(3-(3-fluoro-4-methylphenyl)-propiolyl)-2-methyl-indoline | 294.1 |
| 69 | 2-(3-(4-tert.-butylphenyl)-propiolyl)-1,2,3,4-tetrahydro-isoquinoline | 318.2 |
| 70 | 1-(3-(4-tert.-butylphenyl)-propiolyl)-2-methyl-indoline | 318.2 |
| 71 | 2-(3-(3,4-dimethylphenyl)-propiolyl)-1,2,3,4-tetrahydro-isoquinoline | 290.1 |
| 72 | 1-(3-(3,4-dimethylphenyl)-propiolyl)-2-methyl-indoline | 290.1 |
| 73 | 1-(3-(2-bromo-5-methoxyphenyl)-propiolyl)-2-methyl-indoline | 370.0 |
| 74 | 1-(3-(2-tolyl)-propiolyl)-2-methyl-indoline | 276.1 |
| 75 | 1-(3-(2-trifluoromethylphenyl)-propiolyl)-2-methyl-indoline | 330.1 |
| 76 | 2-(3-(2-cyano-phenyl)-propiolyl)-1,2,3,4-tetrahydro-isoquinoline | 287.1 |
| 77 | 2-(3-(3-cyano-phenyl)-propiolyl)-1,2,3,4-tetrahydro-isoquinoline | 287.1 |
| 78 | 1-(3-(2-tolyl)-propiolyl)-1,2,3,4-tetrahydro-quinoline | 276.1 |
| 79 | 1-(3-(2-trifluoromethylphenyl)-propiolyl)-1,2,3,4-tetrahydro-quinoline | 330.1 |

### Pharmacological Data:

1. The affinity of the substituted propiolic acid amides according to the invention of the general formula I for the mGluR5 receptor was determined as described above (Pharmacological methods I and II). The substituted propiolic acid amides according to the invention exhibit an excellent affinity for the mGluR5 receptor. Table 3 below reproduces the pharmacological data for substituted propiolic acid amides:

TABLE 3

| | | IC₅₀ mGluR5 receptor | |
|---|---|---|---|
| | mGluR5 receptor | (pig) [³H]- | Kᵢ mGluR5 receptor |
| | (pig) (10 μM) | MPEP binding | (human) |
| Ex. | Inhibition (%) | [μM] | Ca²⁺ influx [nM] |
| 1 | | 0.180 | 11.5 |
| 2 | | 1.750 | |
| 3 | | 7.940 | |
| 4 | | 2.320 | |
| 5 | | 0.023 | 1.0 |
| 6 | | 1.820 | |
| 7 | | 8.570 | |
| 8 | | 3.550 | |
| 9 | | 2.360 | |
| 10 | 16 | | |
| 11 | | 2.7200 | |
| 12 | | 0.0230 | 0.3 |
| 13 | | 2.5400 | |
| 14 | | 3.1000 | |
| 15 | | 0.7200 | |
| 16 | | 0.7200 | |
| 17 | | 0.2400 | |
| 18 | | 0.0054 | 1.2 |
| 19 | | 0.0530 | 4.9 |
| 20 | | 0.0550 | |
| 21 | | 0.1100 | |
| 22 | | 0.0760 | |
| 23 | | 0.3200 | |
| 24 | | 0.0045 | |
| 25 | | 0.0047 | |
| 26 | | 0.0047 | |
| 27 | | 0.0260 | |
| 28 | | 0.0870 | |
| 29 | | 0.0084 | |
| 30 | | 0.0015 | 0.9 |
| 31 | | 0.0026 | |
| 33 | 27 | | |
| 34 | 12 | | |
| 35 | 77 | | |
| 36 | 27 | | |
| 37 | 83 | | |
| 38 | 49 | | |
| 39 | 44 | | |
| 40 | 34 | | |
| 41 | 16 | | |
| 43 | 84 | | |
| 44 | 39 | | |
| 46 | 18 | | |
| 50 | 16 | | |
| 51 | 103 | | |
| 52 | 68 | | |
| 53 | 24 | | |
| 54 | 46 | | |
| 55 | 86 | | |
| 56 | 48 | | |
| 57 | 18 | | |
| 63 | 34 | | |
| 64 | 88 | | |
| 65 | 34 | | |
| 66 | 70 | | |
| 67 | 12 | | |
| 68 | 11 | | |
| 71 | 43 | | |
| 72 | 25 | | |
| 73 | 45 | | |
| 74 | 12 | | |
| 75 | 45 | | |
| 76 | 26 | | |
| 77 | 90 | | |

2. The substituted propiolic acid amides according to the invention effectively inhibit the pain response in rats in the formaldehyde test.

| | | Formaldehyde test (rat) per os |
|---|---|---|
| | ED₅₀ formaldehyde test | Reduction in nociceptive behaviour |
| Ex. | (rat) i.v. [mg/kg] | relative to control at 10 mg/kg [%] |
| 18 | 1.06 | 41 |

1. A substituted propiolic acid amides of the formula I, 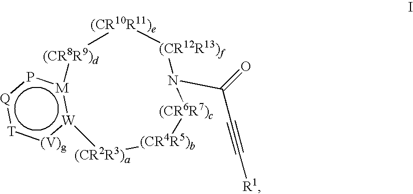
in which
R¹ denotes unsubstituted or substituted aryl or unsubstituted or substituted heteroaryl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
or R² and R³ or R⁴ and R⁵ or R⁶ and R⁷ or R⁸ and R⁹ or R¹⁰ and R¹¹ or R¹² and R¹³ in each case together denote an oxo group (═O) or a thioxo group (═S);
M and W, mutually independently, in each case denote N or C;
P denotes CR¹⁴, N, NR¹⁵, O or S;
Q denotes CR¹⁶, N, NR¹⁷, O or S;
T denotes CR¹⁸, N, NR¹⁹, O or S;
V denotes CR²⁰, N, NR²¹, O or S;
g denotes 0 or 1:
wherein M, W; P, Q, T, V and g together form a 5- or 6-membered aromatic or heteroaromatic ring;
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; —NH—C(═O)—NH—R³⁸; —NH—C(═S)—NH—R³⁹; —NH—S(═O)₂—R⁴⁰; —NR⁴¹—S(═O)₂—R⁴²; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
R¹⁵, R¹⁷, R¹⁹ and R²¹, mutually independently, in each case denote H; —C(═O)—OH; —C(═O)—H; —C(═O)—R²⁷; —C(═O)—O—R²⁸; —C(═O)—NH₂; —C(═O)—NH—R³³; —C(═O)—NR³⁴R³⁵; —S(═O)—R³⁶; —S(═O)₂—R³⁷; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
and R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴², mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
with the proviso that compounds are excluded in which the sum of a, b, c, d, e and f is equal to 3, W and M in each case denote C, P denotes CR¹⁴, Q denotes CR¹⁶, T denotes CR¹⁸ and V denotes CR²⁰
with the proviso that compounds are excluded in which g is 0, W and M in each case denote C, P denotes NR¹⁵, Q denotes CR¹⁶ and T denotes N;
and with the proviso that compounds are excluded in which g is 0, W and M in each case denote C, P denotes N, Q denotes CR¹⁶ and T denotes NR¹⁹;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

2. A compound according to claim 1, wherein
R¹ denotes unsubstituted or substituted aryl or unsubstituted or substituted heteroaryl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
or R² and R³ or R⁴ and R⁵ or R⁶ and R⁷ or R⁸ and R⁹ or R¹⁰ and R¹¹ or R¹² and R¹³ in each case together denote an oxo group (═O) or a thioxo group (═S);
M and W, mutually independently, in each case denote N or C;
P denotes CR¹⁴, N, NR¹⁵, O or S;
Q denotes CR¹⁶, N, NR¹⁷, O or S;
T denotes CR¹⁸, N, NR¹⁹, O or S;
V denotes CR²⁰, N, NR²¹, O or S;
g denotes 0 or 1:
wherein M, W; P, Q, T, V and g together form a 5- or 6-membered aromatic or heteroaromatic ring;
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; —NH—C(═O)—NH—R³⁸; —NH—C(═S)—NH—R³⁹; —NH—S(═O)₂—R⁴⁰; —NR⁴¹—S(═O)₂—R⁴²; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
R¹⁵, R¹⁷, R¹⁹ and R²¹, mutually independently, in each case denote H; —C(═O)—OH; —C(═O)—H; —C(═O)—R²⁷; —C(═O)—O—R²⁸; —C(═O)—NH₂; —C(═O)—NH—R³³; —C(═O)—NR³⁴R³⁵; —S(═O)—R³⁶; —S(═O)₂—R³⁷; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
and R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴², mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
with the proviso that compounds are excluded in which the sum of a, b, c, d, e and f is equal to 3, W and M in each case denote C, P denotes CR¹⁴, Q denotes CR¹⁶, T denotes CR¹⁸ and V denotes CR²⁰;
with the proviso that compounds are excluded in which g is 0, W and M in each case denote C, P denotes NR¹⁵, Q denotes CR¹⁶ and T denotes N;
and with the proviso that compounds are excluded in which g is 0, W and M in each case denote C, P denotes N, Q denotes CR¹⁶ and T denotes NR¹⁹;
wherein
the above-stated alkyl residues are in each case branched or straight-chain and comprise 1, 2, 3, 4, 5, 6, 7, 8, 9, 10, 11 or 12 carbon atoms as chain links;
the above-stated alkenyl residues are in each case branched or straight-chain and comprise 2, 3, 4, 5, 6, 7, 8, 9, 10, 11 or 12 carbon atoms as chain links;
the above-stated alkynyl residues are in each case branched or straight-chain and comprise 2, 3, 4, 5, 6, 7, 8, 9, 10, 11 or 12 carbon atoms as chain links;
the above-stated heteroalkyl residues, heteroalkenyl residues and heteroalkynyl residues are in each case 2-, 3-, 4-, 5-, 6-, 7-, 8-, 9-, 10-, 11- or 12-membered;
the above-stated heteroalkyl residues, heteroalkenyl residues and heteroalkynyl residues in each case optionally comprise 1, 2 or 3 heteroatom(s) mutually independently selected from the group consisting of oxygen, nitrogen (NH) and sulfur as chain link(s);
the above-stated alkyl residues, alkenyl residues, alkynyl residues, heteroalkyl residues, heteroalkenyl residues and heteroalkynyl residues may be substituted in each case with optionally 1, 2, 3, 4 or 5 substituents mutually independently selected from the group consisting of F, Cl, Br, I, —NO₂, —CN, —OH, —SH, —NH₂, —N(C₁₋₅-alkyl)₂, —N(C₁₋₁₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the phenyl residues may be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl;
the above-stated cycloalkyl residues in each case comprise 3, 4, 5, 6, 7, 8 or 9 carbon atoms as ring members;
the above-stated cycloalkenyl residues in each case comprise 3, 4, 5, 6, 7, 8 or 9 carbon atoms as ring members;
the above-stated heterocycloalkyl residues are in each case 3-, 4-, 5-, 6-, 7-, 8- or 9-membered;
the above-stated heterocycloalkenyl residues are in each case 4-, 5-, 6-, 7-, 8- or 9-membered;
the above-stated heterocycloalkyl residues and heterocycloalkenyl residues in each case optionally comprise 1, 2 or 3 heteroatom(s) mutually independently selected from the group consisting of oxygen, nitrogen (NH) and sulfur as ring member(s);
the above-stated cycloalkyl residues, heterocycloalkyl residues, cycloalkenyl residues or heterocycloalkenyl residues may be substituted in each case with optionally 1, 2, 3, 4 or 5 substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C═C—Si(CH₃)₃, —C═C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —C(═O)—H, —C(═O)—C₁₋₅-alkyl, —C(═O)—NH₂, —C(═O)—N(C₁₋₅-alkyl)₂, —C(═O)—N(H)(—C₁₋₅-alkyl) and phenyl, wherein the phenyl residue may in each case be unsubstituted or substituted with 1, 2, 3, 4 or 5 substituents mutually independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅ alkyl, —C₂₋₅ alkenyl, —C₂₋₅ alkynyl, —C≡C—Si(CH₃)₃, —C≡C—Si(C₂H₅)₃, —C(═O)—O—C₁₋₅-alkyl, and —C(═O)—CF₃, wherein the above-stated phenyl residues may be substituted with 1, 2, 3, 4 or 5 substituents mutually independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert.-butyl;
the above-stated alkylene residues are in each case branched or straight-chain and comprise 1, 2, 3, 4, 5, 6, 7, 8, 9, 10, 11 or 12 carbon atoms as chain links;
the above-stated alkenylene residues are in each case branched or straight-chain and comprise 2, 3, 4, 5, 6, 7, 8, 9, 10, 11 or 12 carbon atoms as chain links;
the above-stated alkynylene residues are in each case branched or straight-chain and comprise 2, 3, 4, 5, 6, 7, 8, 9, 10, 11 or 12 carbon atoms as chain links;
the above-stated heteroalkylene residues and heteroalkenylene residues are in each case 2-, 3-, 4-, 5-, 6-, 7-, 8-, 9-, 10-, 11- or 12-membered;
the above-stated heteroalkylene and heteroalkenylene groups may in each case optionally comprise 1, 2 or 3 heteroatom(s) mutually independently selected from the group consisting of oxygen, nitrogen (NH) and sulfur as chain link(s);
the above-stated alkylene, alkenylene, alkynylene, heteroalkylene or heteroalkenylene group may in each case be unsubstituted or substituted with optionally 1, 2, 3, 4 or 5 substituents mutually independently selected from the group consisting of phenyl, F, Cl, Br, I, —NO₂, —CN, —OH, —O-phenyl, —O—CH₂-phenyl, —SH, —S-phenyl, —S—CH₂-phenyl, NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₁₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the phenyl residues may be substituted with 1, 2, 3, 4 or 5 substituents mutually independently selected from the group consisting of F, Cl, Br, I, —CN, —NO₂, —OH, —SH, —NH₂, —C(═O)—OH, —C₁₋₁₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
the above-stated aryl residues are mono- or bicyclic and comprise 6, 10 or 14 carbon atoms;
the above-stated heteroaryl residues are mono-, di- or tricyclic and 5-, 6-, 7-, 8-, 9-, 10-, 11-, 12-, 13- or 14-membered;
the above-stated 5- to 14-membered heteroalkyl residues optionally comprise 1, 2, 3, 4 or 5 heteroatom(s) mutually independently selected from the group consisting of oxygen, nitrogen (NH) and sulfur as ring member(s);
and the above-stated aryl or heteroaryl residues may optionally in each case be substituted with 1, 2, 3, 4 or 5 substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C—C≡Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═OC)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, cyclopropyl, cyclobutyl, cyclopentyl, cyclohexyl, pyrazolyl, phenyl, furyl (furanyl), thiazolyl, thiadiazolyl, thiophenyl (thienyl), benzyl and phenethyl, wherein the cyclic substituents or the cyclic residues of these substituents may themselves be substituted with optionally 1, 2, 3, 4 or 5 substituents mutually independently selected from the group consisting of F, Cl, Br, I, —CN, —NO₂, —OH, —SH, —NH₂, —C(═O)—OH, —C₁₋₅ alkyl, —(CH₂)—O—C₁₋₅-alkyl, —C₂₋₅ alkenyl, —C₂₋₅ alkynyl, —C≡C—Si(CH₃)₃, —C═C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvates.

3. A compounds according to claim 1, wherein
R¹ denotes a residue selected from the group consisting of phenyl, naphthyl, anthracenyl, furyl, thienyl, pyrazolyl, pyridazinyl, pyrazinyl, pyrimidinyl, pyridinyl, pyrrolyl, oxazolyl, isoxazolyl, thiazolyl, thiadiazolyl, oxadiazolyl, triazolyl, imidazolyl, indolyl, benzo[b]thiophenyl, benzo[d]thiazolyl, benzo[b]furanyl, quinolinyl, isoquinolinyl and quinazolinyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C≡C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl.

4. A compound according to claim 1, wherein
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₂₋₆ heteroalkyl, C₂₋₆ heteroalkenyl or C₂₋₆ heteroalkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂, or comprises 1 or 2 heteroatom(s) mutually independently selected from the group consisting of oxygen, sulfur and nitrogen (NH) as chain link(s); or C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
or R² and R³ or R⁴ and R⁵ or R⁶ and R⁷ or R⁸ and R⁹ or R¹⁰ and R¹¹ or R¹² and R¹³, mutually independently, together in each case denote a residue selected from the group consisting of an oxo group (═O) or a thioxo group (═S).

5. A compound according to claim 1, wherein W, M, P, Q, T and optionally V together denote a ring selected from the group consisting of

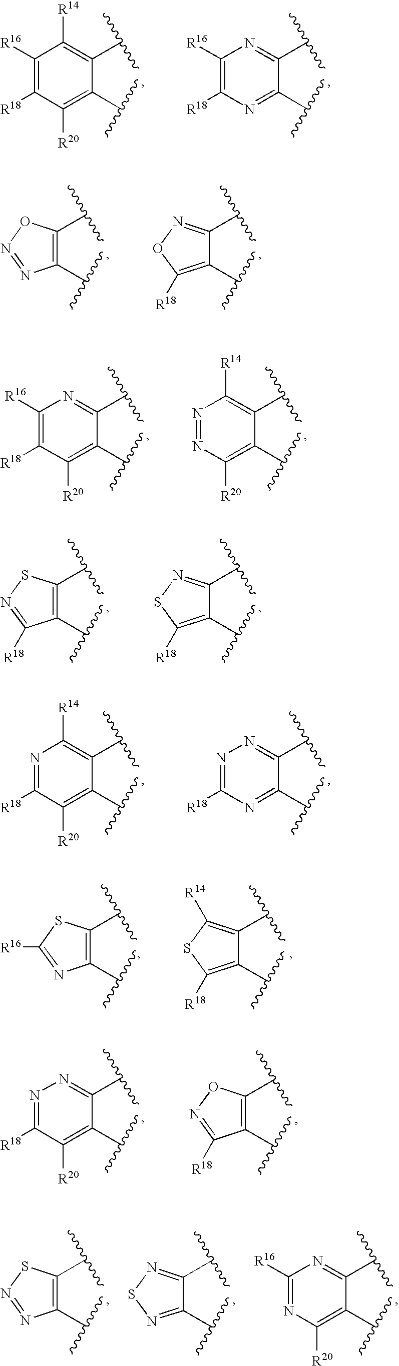

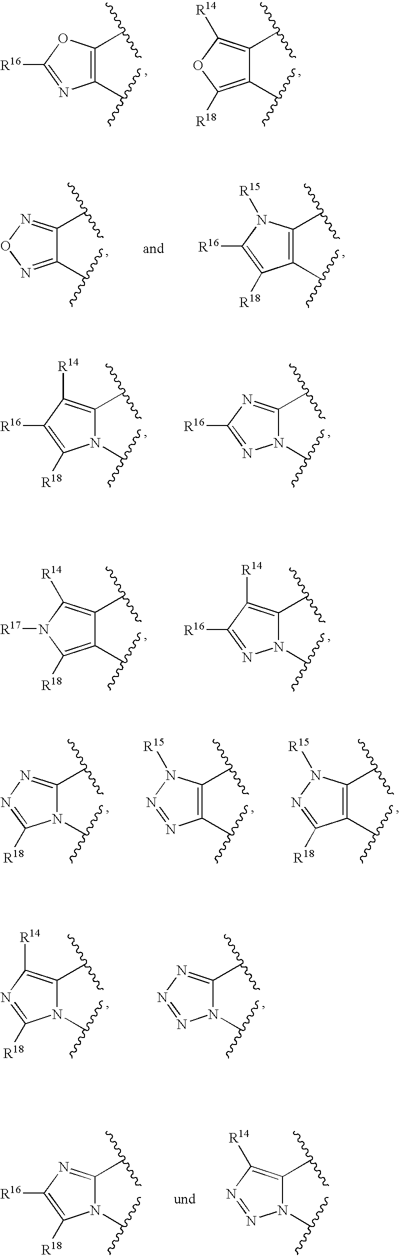

6. A compound according to claim 1, wherein
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃.

7. A compound according to claim 1, wherein R¹⁵, R¹⁷, R¹⁹ and R²¹, mutually independently, in each case denote H; —C(═O)—H; —C(═O)—R²⁷; —C(═O)—O—R²⁸; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃.

8. A compound according to claim 1, wherein R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴² mutually independently, in each case denote C₁₋₆ alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, pyrrolyl, indolyl, furanyl, benzo[b]furanyl, thiophenyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅.

9. A compound according to claim 1, wherein
R¹ denotes a residue selected from the group consisting of phenyl, naphthyl, anthracenyl, furyl, thienyl, pyrazolyl, pyridazinyl, pyrazinyl, pyrimidinyl, pyridinyl, pyrrolyl, oxazolyl, isoxazolyl, thiazolyl, thiadiazolyl, oxadiazolyl, triazolyl, imidazolyl, indolyl, benzo[b]thiophenyl, benzo[d]thiazolyl, benzo[b]furanyl, quinolinyl, isoquinolinyl and quinazolinyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C≡C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₂₋₆ heteroalkyl, C₂₋₆ heteroalkenyl or C₂₋₆ heteroalkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂ or comprises 1 or 2 heteroatom(s) mutually independently selected from the group consisting of oxygen, sulfur and nitrogen (NH) as chain link(s); or C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
or R² and R³ or R⁴ and R⁵ or R⁶ and R⁷ or R³ and R⁹ or R¹⁰ and R¹¹ or R¹² and R¹³, mutually independently, together in each case denote a residue selected from the group consisting of an oxo group (═O) or a thioxo group (═S;
g denotes 0 or 1;
W, M, P, Q, T and optionally V together denote a ring selected from the group consisting of 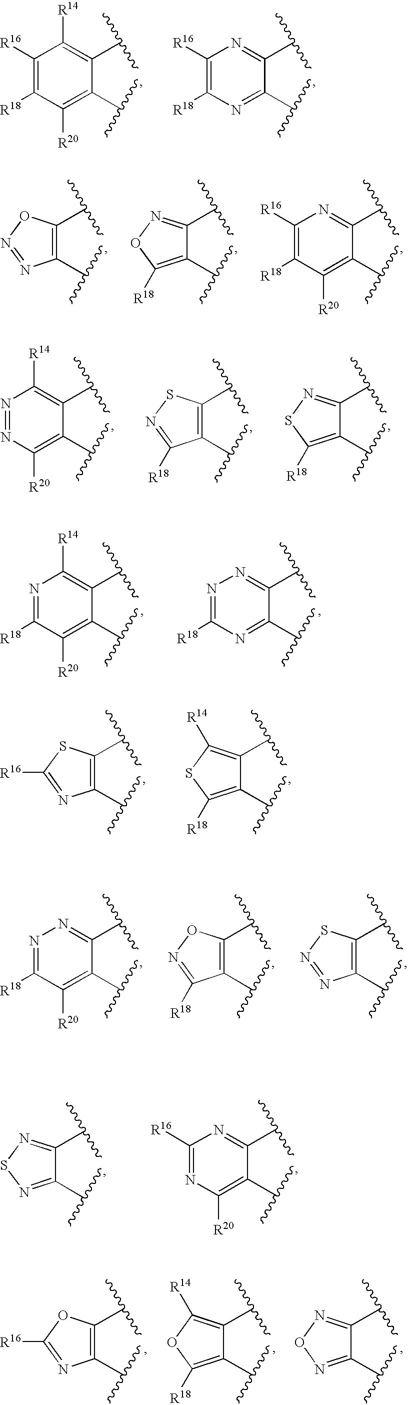
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, 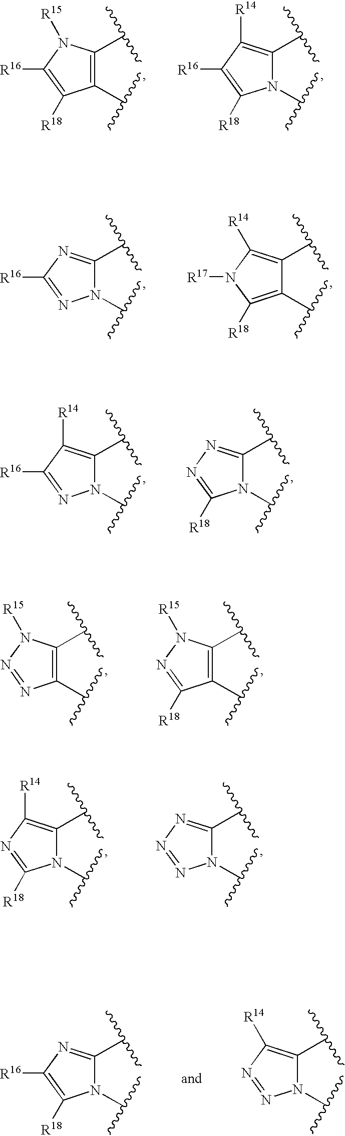
oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
R¹⁵ and R¹⁷, mutually independently, in each case denote H; C(═O)—H; —C(═O)—R²⁷; —C(═O)—O—R²⁸; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group and/or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
and R²², R²³, R²⁴, R²⁵, R²⁶, R²⁷, R²⁸, R²⁹, R³⁰, R³¹, R³², R³³, R³⁴, R³⁵, R³⁶ and R³⁷, mutually independently, in each case denote C₁₋₆ alkyl, which is unsubstituted or substituted with optionally 1, 2, 3, 4 or 5 substituents mutually independently selected from the group consisting of F, Cl, Br, I, —NO₂, —CN, —OH, —SH and —NH₂; C₃₋₇ cycloalkyl, C₅₋₆ cycloalkenyl, 5- to 7-membered heterocycloalkyl and 5- to 7-membered heterocycloalkenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or denote a residue selected from the group consisting of phenyl, naphthyl, anthracenyl, pyrrolyl, indolyl, furanyl, benzo[b]furanyl, thiophenyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
with the proviso that compounds are excluded in which the sum of a, b, c, d, e and f is equal to 3 and W, M, P, Q, T and V together denote 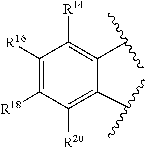
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

10. A compound according to claim 1, wherein
R¹ denotes a residue selected from the group consisting of phenyl, thienyl, pyridazinyl, pyrazinyl, pyrimidinyl, pyridinyl, thiazolyl and thiadiazolyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C≡C—Si(CH₃)₃, —C═C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl;
a, b, c, d, e and f in each case denote 0 or 1; wherein the sum of a, b, c, d, e and f is equal to 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; —CF₃; —CF₂H; —CFH₂; —C₂F₅; —CH₂—CF₃; —CH₂—OH; —CH₂—NH₂; —CH₂—CN; —CH₂—CH₂—OH; —CH₂—CH₂—NH₂; —CH₂—CH₂—CN; —CH₂—CH₂—CH₂—OH; C₁₋₆ alkyl, C₂₋₆ alkenyl or C₂₋₆ alkynyl, which is in each case unsubstituted; C₂₋₆ heteroalkyl, C₂₋₆ heteroalkenyl or C₂₋₆ heteroalkynyl, which is in each case unsubstituted or comprises 1 or 2 heteroatom(s) mutually independently selected from the group consisting of oxygen, sulfur and nitrogen (NH) as chain link(s); or C₃₋₇ cycloalkyl, C₅₋₆ cycloalkenyl, 5- to 7-membered heterocycloalkyl and 5- to 7-membered heterocycloalkenyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅;
g denotes 0 or 1;
W, M, P, Q, T and optionally V together denote a ring selected from the group consisting of 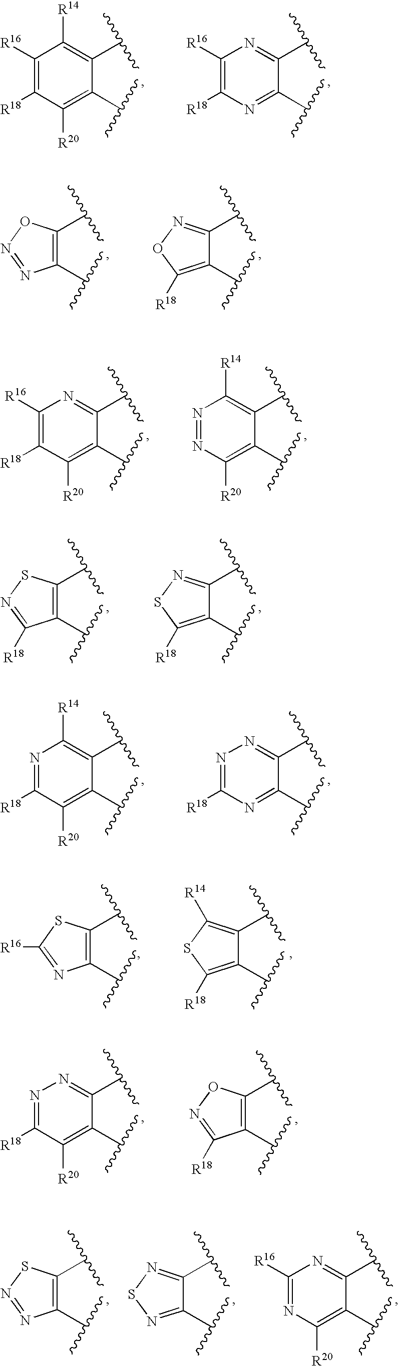 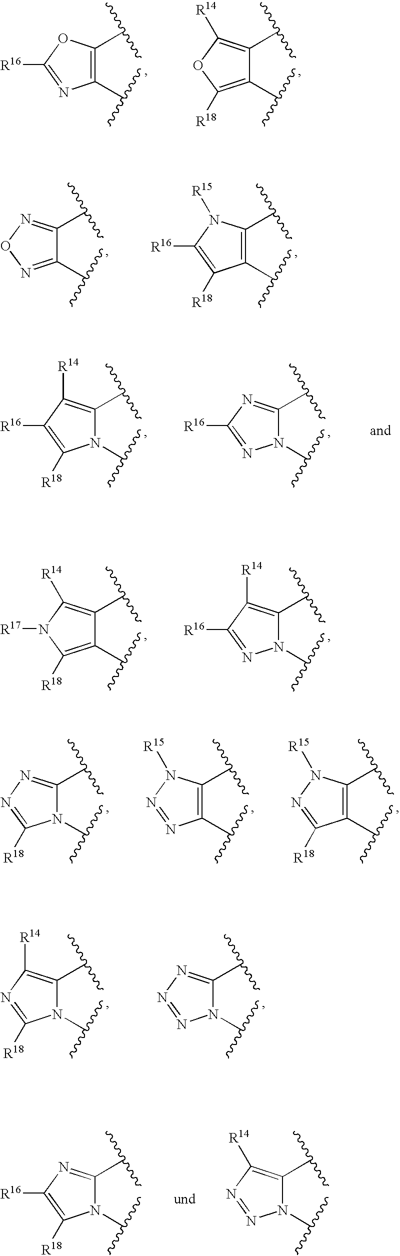
R¹⁴, R¹⁶, R¹⁸ and R²⁰, mutually independently, in each case denote H; F; Cl; Br; I; —CF₃; —CF₂H; —CFH₂; —C₂F₅; —CH₂—CF₃; —NO₂; —CN; —NH₂; —OH; —SH; —C(═O)—OH; —C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted; C₃₋₇ cycloalkyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —O—CH₃, —O—C₂H₅ and —O—C₃H₇; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
R¹⁵ and R¹⁷, mutually independently, in each case denote H; —CF₃; —CF₂H; —CFH₂; —C₂F₅; —CH₂—CF₃; —C(═O)—H; —C(═O)—R²⁷; —C(═O)—O—R²⁸; —C(═O)—NH—R³³; —C(═O)—NR³⁴R³⁵; —S(═O)—R³⁶; —S(═O)₂—R³⁷; C₁₋₆ alkyl, C₂₋₆ alkenyl or C₂₋₆ alkynyl, which is in each case unsubstituted; C₃₋₇ cycloalkyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —O—CH₃, —O—C₂H₅ and —O—C₃H₇; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
and R²², R²³, R²⁴, R²⁵, R²⁶, R²⁷, R²⁸, R²⁹, R³⁰, R³¹, R³², R³³, R³⁴, R³⁵, R³⁶ and R³⁷, mutually independently, in each case denote —CF₃; —CF₂H; —CFH₂; —C₂F₅; —CH₂—CF₃; unsubstituted C₁₋₆ alkyl; C₃₋₇ cycloalkyl, C₅₋₆ cycloalkenyl, 5- to 7-membered heterocycloalkyl and 5- to 7-membered heterocycloalkenyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅ and —O—C₃H₇; or denote a residue selected from the group consisting of phenyl, naphthyl, furanyl, thiophenyl, pyrazolyl, imidazolyl, thiazolyl, thiadiazolyl, triazolyl, oxazolyl, oxadiazolyl, isoxazolyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
with the proviso that compounds are excluded in which the sum of a, b, c, d, e and f is equal to 3 and W, M, P, Q, T and V together denote 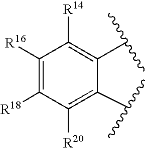
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

11. A compound according to claim 1, wherein
R¹ denotes a residue selected from the group consisting of phenyl, pyridinyl and thienyl, which is in each case unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, cyclobutyl, cyclopentyl, cyclohexyl, —C═C—Si(CH₃)₃, —C≡C—Si(C₂H₅)₃, —CH₂—O—CH₃, —CH₂—O—C₂H₅, —OH, —O—CH₃, —O—C₂H₅, —O—C₃H₇, —S—CH₃, —S—C₂H₅, —S(═O)—CH₃, —S(═O)₂—CH₃, —S(═O)—C₂H₅, —S(═O)₂—C₂H₅, —NH₂, —N(CH₃)₂, —N(C₂H₅)₂, —NH—CH₃, —NH—C₂H₅, —NO₂, —CF₃, —CH₂F, —CHF₂, —O—CF₃, —S—CF₃, —SH, —NH—S(═O)₂—CH₃, —C(═O)—OH, —C(═O)—H; —C(═O)—CH₃, —C(═O)—C₂H₅, —C(═O)—NH₂, —C(═O)—N(CH₃)₂, —C(═O)—NH—CH₃, —NH—C(═O)—CH₃, —NH—C(═O)—C₂H₅, —C(═O)—O—CH₃, —C(═O)—O—C₂H₅, —C(═O)—O—C(CH₃)₃ and phenyl;
a, b, c, d, e and f in each case denote 0 or 1; wherein the sum of a, b, c, d, e and f is equal to 2, 3, 4 or 5;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; —CF₃; —CF₂H; —CFH₂; —C₂F₅; —CH₂—CF₃; —CH₂—OH; —CH₂—NH₂; —CH₂—CN; —CH₂—CH₂—OH; —CH₂—CH₂—NH₂; —CH₂—CH₂—CN; —CH₂—CH₂—CH₂—OH; C₁₋₆ alkyl, C₂₋₆ alkenyl or C₂₋₆ alkynyl, which is in each case unsubstituted; or denote unsubstituted C₃₋₇ cycloalkyl;
g denotes 0 or 1;
W, M, P, Q, T and optionally V together denote a ring selected from the group consisting of 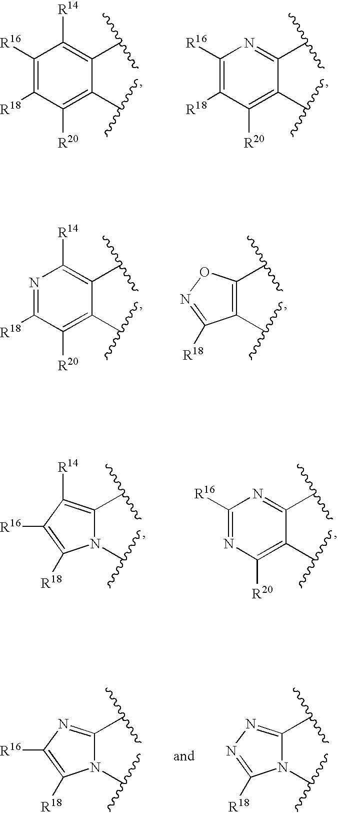
R¹⁴, R¹⁶, R¹⁸ and R²⁰, mutually independently, in each case denote H; F; Cl; Br; I; —CF₃; —CF₂H; —CFH₂; —C₂F₅; —CH₂—CF₃; —NO₂; —CN; —NH₂; —OH; —C(═O)—OH; —C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NH—C(═O)—R³⁰; —NR³¹—C(═O)—R³²; —C(═O)—NH₂; —C(═O)—NH—R³³; —C(═O)—NR³⁴R³⁵; C₁₋₆ alkyl, C₂₋₆ alkenyl or C₂₋₆ alkynyl, which is in each case unsubstituted; unsubstituted C₃₋₇ cycloalkyl; or denote a residue selected from the group consisting of phenyl, naphthyl, anthracenyl, thienyl, furyl, pyridinyl, thiazolyl, thiadiazolyl, oxazolyl, oxadiazolyl and isoxazolyl, which may in each case be attached via a C₁₋₃ alkylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, isobutyl, 2-butyl, tert.-butyl, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —CF₃, —CHF₂, —CH₂F and —O—CF₃;
R²², R²³, R²⁴, R²⁷, R²⁸, R²⁹, R³⁰, R³¹, R³², R³³, R³⁴ and R³⁵, mutually independently, in each case denote —CF₃; —CF₂H; —CFH₂; —C₂F₅; —CH₂—CF₃; or denote unsubstituted C₁₋₆ alkyl;
and R²⁵ and R²⁶, mutually independently, in each case denote CF₃; —CF₂H; —CFH₂; —C₂F₅; —CH₂—CF₃; unsubstituted C₁₋₆ alkyl; or denote a residue selected from the group consisting of phenyl, naphthyl, furanyl, pyrazolyl, imidazolyl, thiazolyl, thiadiazolyl, triazolyl, oxazolyl, oxadiazolyl, isoxazolyl and thiophenyl, which may in each case be attached via a C₁₋₃ alkylene, C₂₋₃ alkenylene or C₂₋₃ alkynylene group or is unsubstituted or substituted with optionally 1, 2, 3, 4 or 5 substituents mutually independently selected from the group consisting of F, Cl, Br, I, —CN, methyl, ethyl, n-propyl, isopropyl, n-butyl, 2-butyl, isobutyl, tert.-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
with the proviso that compounds are excluded in which the sum of a, b, c, d, e and f is equal to 3 and W, M, P, Q, T and V together denote 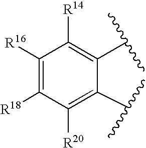
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

12. A compound according to claim 1, wherein
R¹ denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
a, b, c, d, e and f in each case denote 0 or 1; wherein the sum of a, b, c, d, e and f is equal to 2, 3 or 4;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
g denotes 0 or 1;
W, M, P, Q, T and optionally V together denote a ring selected from the group consisting of 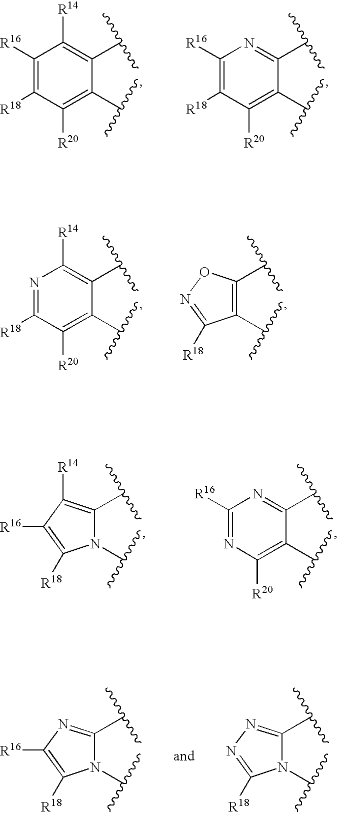
and R¹⁴, R¹⁶, R¹⁸ and R²⁰, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
with the proviso that compounds are excluded in which the sum of a, b, c, d, e and f is equal to 3 and W, M, P, Q, T and V together denote 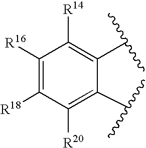
in each case optionally in the form of one of the pure stereoisomers thereof, in the form of one of the racemates thereof or in the form of a mixture of stereoisomers in any mixing ratio, or in each case in the form of a corresponding salts or in each case in the form of a corresponding solvate.

13. A compound according to claim 1, wherein
R¹ denotes a residue selected from the group consisting of phenyl, thienyl and pyridinyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
a, b, c, d, e and f in each case denote 0 or 1; wherein the sum of a, b, c, d, e and f is equal to 2, 3 or 4;
R² and R⁴ mutually independently, in each case denote H; methyl or ethyl;
R³, R⁵, R⁶, R⁷, R⁸, R⁹, R¹⁰, R¹¹, R¹² and R¹³ in each case denote H;
g denotes 0 or 1;
W, M, P, Q, T and optionally V together denote a ring selected from the group consisting of 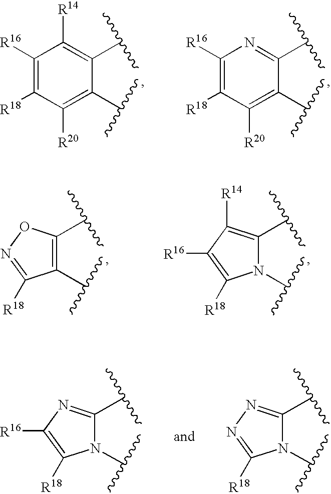
R¹⁴, R¹⁶, R¹⁸ and R²⁰, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl;
with the proviso that compounds are excluded in which the sum of a, b, c, d, e and f is equal to 3 and W, M, P, Q, T and V together denote 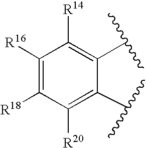
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

14. A compound of the formula Ia, according to claim 1, 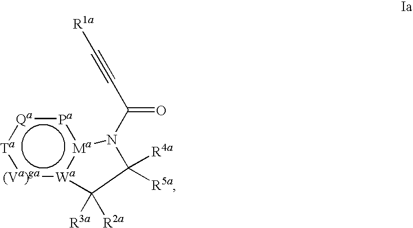
in which
R^{1a} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2a}, R^{3a}, R^{4a} and R^{5a}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
ga denotes 0 or 1;
W^{a}, M^{a}, P^{a}, Q^{a}, T^{a} and optionally V^{a} together denote a ring selected from the group consisting of 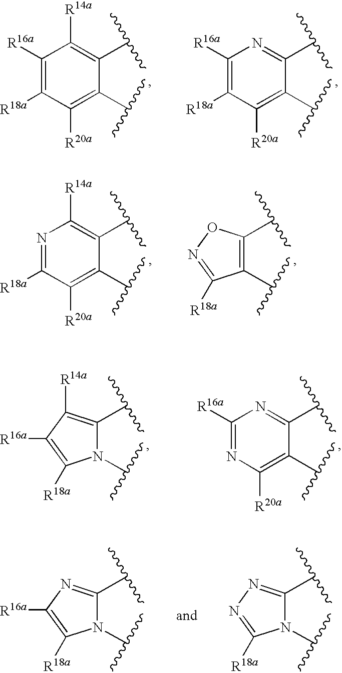
and R^{14a}, R^{16a}, R^{18a} and R^{20a}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

15. A compound according to claim 14, wherein
R^{1a} denotes a residue selected from the group consisting of phenyl, thienyl and pyridinyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
R^{2a} and R^{4a}, mutually independently, in each case denote H; methyl or ethyl;
R^{3a} and R^{5a}, in each case denote H;
ga denotes 0 or 1;
W^{a}, M^{a}, P^{a}, Q^{a}, T^{a} and optionally V^{a} together denote a ring selected from the group consisting of 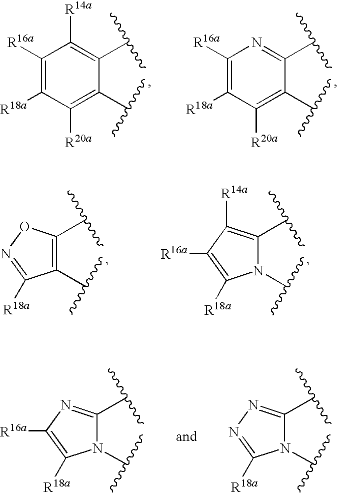
and R^{14a}, R^{16a}, R^{18a} and R^{20a}, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl.

16. A compound of the formula Ib, according to claim 1, 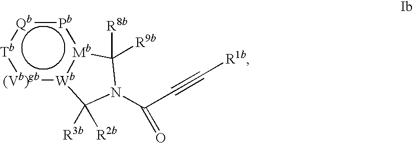
in which
R^{1b} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2b}, R^{3b}, R^{8b} and R^{9b}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and cyclobutyl;
gb denotes 0 or 1;
W^{b}, M^{b}, P^{b}, Q^{b}, T^{b} and optionally V^{b} together denote a ring selected from the group consisting of 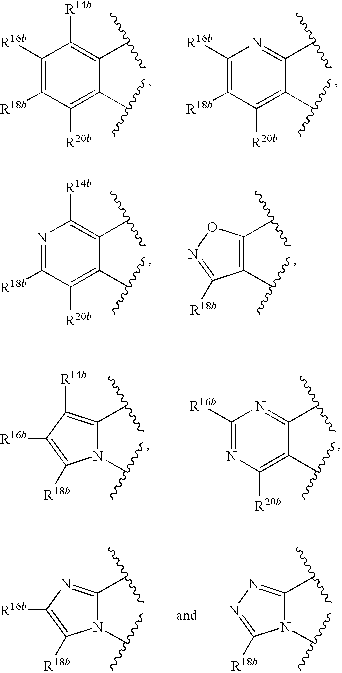
and R^{14b}, R^{16b}, R^{18b} and R^{20b}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

17. A compound according to claim 16, wherein
R^{1b} denotes a residue selected from the group consisting of phenyl, pyridinyl and thienyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
R^{2b}, R^{3b}, R^{4b} and R^{5b}, mutually independently, in each case denote H; methyl or ethyl;
gb denotes 0 or 1;
W^{b}, M^{b}, P^{b}, Q^{b}, T^{b} and optionally V^{b} together denote a ring selected from the group consisting of 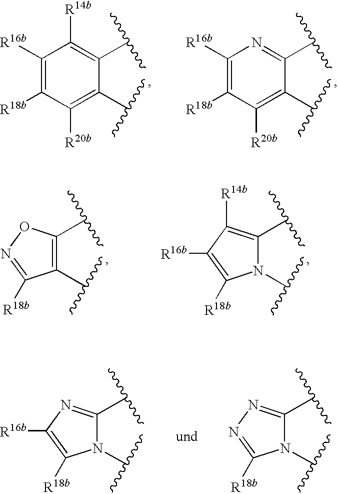
and R^{14b}, R^{16b}, R^{18b} and R^{20b}, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl.

18. A compound of the formula Ic, according to claim 1, 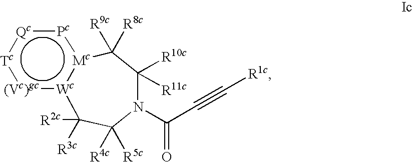
in which
R^{1c} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2c}, R^{3c}, R^{4c}, R^{5c}, R^{8c}, R^{9c}, R^{10c} and R^{11c}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
gc denotes 0 or 1;
W^{c}, M^{c}, P^{c}, Q^{c}, T^{c} and optionally V^{c} together denote a ring selected from the group consisting of 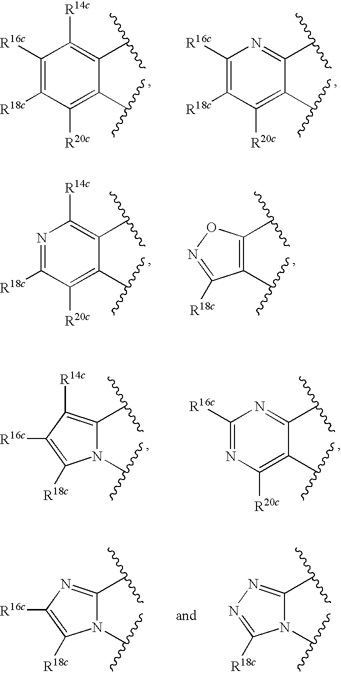
and R^{14c}, R^{16c}, R^{18c} and R^{20c}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—
C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

19. A compound according to claim 18, wherein
R^{1c} denotes a residue selected from the group consisting of phenyl, pyridinyl and thienyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
R^{2a}, R^{3c}, R^{4c}, R^{5c}, R^{8c}, R^{9c}, R^{10c} and R^{11c}, mutually independently, in each
case denote H; methyl or ethyl;
gc denotes 0 or 1;
W^{c}, M^{c}, P^{c}, Q^{c}, T^{c} and optionally V^{c} together denote a ring selected from the group consisting of 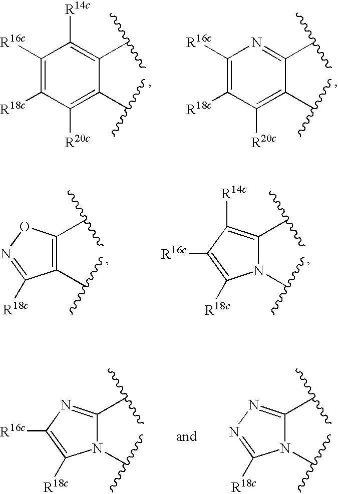
and R^{14c}, R^{16c}, R^{18c} and R^{20c}, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl.

20. A compound of the formula Id, according to claim 1, 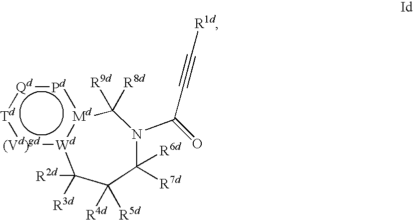
in which
R^{1d} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2d}, R^{3d}, R^{4d}, R^{5d}, R^{6d}, R^{7d}, R^{8d} and R^{9d}, mutually independently, in each case denote H; F; Cl; Br; i; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl and or cyclobutyl;
gd denotes 0 or 1;
W^{d}, M^{d}, P^{d}, Q^{d}, T^{d} and optionally V^{d} together denote a ring selected from the group consisting of 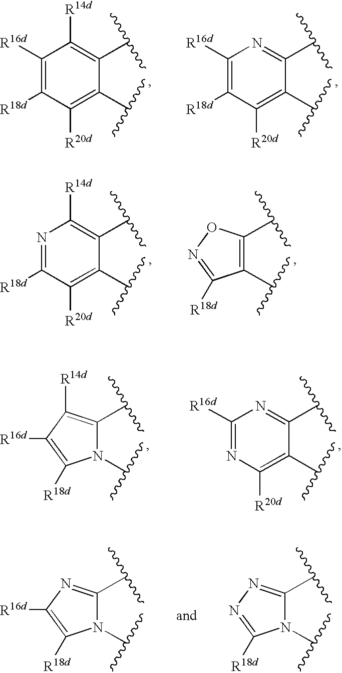
and R^{14d}, R^{16d}, R^{18d} and R^{20d}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

21. A compound according to claim 20, wherein
R^{1d} denotes a residue selected from the group consisting of phenyl, pyridinyl and thienyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
R^{2d}, R^{3d}, R^{4d}, R^{5d}, R^{6d}, R^{7d}, R^{8d} and R^{9d}, mutually independently, in each case denote H; methyl or ethyl;
gd denotes 0 or 1;
W^{d}, M^{d}, P^{d}, Q^{d}, T^{d} and optionally V^{d} together denote a ring selected from the group consisting of 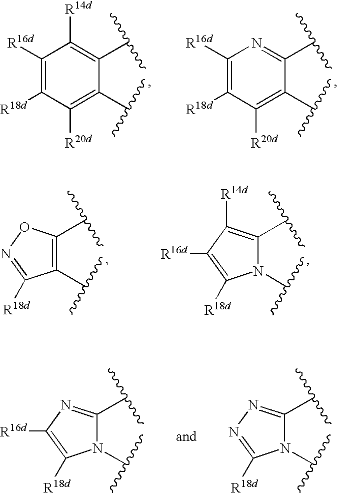
and R^{14a}, R^{16a}, R^{18a} and R^{20a}, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl.

22. A compounds according to claim 1, selected from the group consisting of
[3] 1-(3-phenyl-propiolyl)-2,3-dihydro-indole,
[4] 6-(3-phenyl-propiolyl)-5,6,7,8-tetrahydro-[1,6]naphthyridine,
[6] 2-(3-phenyl-propiolyl)-2,3-dihydro-isoindole,
[8] 3-methyl-5-(3-phenyl-propiolyl)-4,5,6,7-tetrahydro-isoxazolo[4,5-c]pyridine,
[9] 3-phenyl-5-(3-phenyl-propiolyl)-4,5,6,7-tetrahydro-isoxazolo[4,5-c]pyridine,
[10] 7-(3-phenyl-propiolyl)-5,6,7,8-tetrahydro-[1,2,4]triazolo[4,3-a]pyrazine,
3-(3-(3-chlorophenyl)-propiolyl)-1,2,4,5-tetrahydrobenzo[d]azepine,
2-(3-(3-chlorophenyl)-propiolyl)-2,3,4,5-tetrahydro-1H-benzo[c]azepine,
7-(3-(3-chlorophenyl)-propiolyl)-5,6,7,8-tetrahydroimidazo[1,2-a]pyrazine,
2-(3-(3-chlorophenyl)-propiolyl)-1,2,3,4-tetrahydropyrrolo[1,2-a]pyrazine,
1-(3-(2,4-difluorophenyl)-propiolyl)-2-methyl-indoline,
1-(3-(4-fluoro-3-methylphenyl)-propiolyl)-2-methyl-indoline,
1-(3-(2-fluorophenyl)-propiolyl)-2-methyl-indoline, 1-(3-(4-tolyl)-propiolyl)-2-methyl-indoline,
1-(3-(4-trifluoromethylphenyl)-propiolyl)-2-methyl-indoline,
1-(3-phenyl-propiolyl)-2-methyl-indoline,
1-(3-(2,3-dimethylphenyl)-propiolyl)-2-methyl-indoline,
1-(3-(3,5-dimethylphenyl)-propiolyl)-2-methyl-indoline,
1-(3-(2,4-dichlorophenyl)-propiolyl)-2-methyl-indoline,
1-(3-(3-tolyl)-propiolyl)-2-methyl-indoline,
1-(3-(4-methoxyphenyl)-propiolyl)-2-methyl-indoline,
1-(3-(2-thienyl)-propiolyl)-2-methyl-indoline,
1-(3-(2,4-dimethylphenyl)-propiolyl)-2-methyl-indoline,
1-(3-(4-fluorophenyl)-propiolyl)-2-methyl-indoline,
1-(3-(3-chlorophenyl)-propiolyl)-2-methyl-indoline,
1-(3-(3-fluoro-4-methylphenyl)-propiolyl)-2-methyl-indoline,
1-(3-(4-tert.-butylphenyl)-propiolyl)-2-methyl-indoline,
1-(3-(3,4-dimethylphenyl)-propiolyl)-2-methyl-indoline,
1-(3-(2-bromo-5-methoxyphenyl)-propiolyl)-2-methyl-indoline,
1-(3-(2-tolyl)-propiolyl)-2-methyl-indoline and
1-(3-(2-trifluoromethylphenyl)-propiolyl)-2-methyl-indoline;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

23. A compound of the formula Ik, according to claim 1, 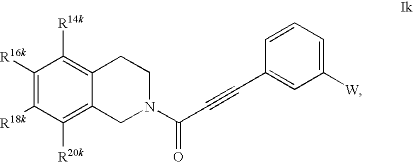
in which
W denotes Cl, methyl or —CN;
and R^{14k}, R^{16k}, R^{18k} and R^{20k}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —NH—C(═O)—CH₃; —N(CH₃)—
C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl or tert.-butyl;
optionally in each case in the form of a corresponding salt or optionally in each case in the form of a corresponding solvate.

24. A compound according to claim 23 selected from the group consisting of
[5] 2-(3-(3-chlorophenyl)-propiolyl)-1 ,2,3,4-tetrahydro-isoquinoline,
2-(3-(3-chlorophenyl)-propiolyl)-6,7-dimethoxy-1,2,3,4-tetrahydro-isoquinoline,
2-(3-(3-chlorophenyl)-propiolyl)-6,7-dihydroxy-1 ,2,3,4-tetrahydro-isoquinoline,
2-(3-(3-chlorophenyl)-propiolyl)-7-hydroxy-6-methoxy-1,2,3,4-tetrahydro-isoquinoline,
2-(3-(3-chlorophenyl)-propiolyl)-6-hydroxy-7-methoxy-1,2,3,4-tetrahydro-isoquinoline,
2-(3-(3-chlorophenyl)-propiolyl)-6-methoxy-1,2,3,4-tetrahydro-isoquinoline,
2-(3-(3-chlorophenyl)-propiolyl)-1-methyl-1,2,3,4-tetrahydro-isoquinoline,
2-(3-(3-chlorophenyl)-propiolyl)-3-methyl-1,2,3,4-tetrahydro-isoquinoline,
2-(3-(3-chlorophenyl)-propiolyl)-6-methyl-1,2,3,4-tetrahydro-isoquinoline,
2-(3-(3-chlorophenyl)-propiolyl)-7-methoxy-1,2,3,4-tetrahydro-isoquinoline,
2-(3-(3-chlorophenyl)-propiolyl)-8-methyl-1,2,3,4-tetrahydro-isoquinoline,
2-(3-(3-chlorophenyl)-propiolyl)-7-methyl-1,2,3,4-tetrahydro-isoquinoline,
2-(3-(3-chlorophenyl)-propiolyl)-5-methyl-1,2,3,4-tetrahydro-isoquinoline,
2-(3-(3-chlorophenyl)-propiolyl)-7-hydroxy-1,2,3,4-tetrahydro-isoquinoline,
[29] 2-(3-(3-chlorophenyl)-propiolyl)-8-methoxy-1,2,3,4-tetrahydro-isoquinoline,
[30] 2-(3-(3-chlorophenyl)-propiolyl)-5-methoxy-1,2,3,4-tetrahydro-isoquinoline,
[31] 2-(3-(3-chlorophenyl)-propiolyl)-1,8-dimethyl-1,2,3,4-tetrahydro-isoquinoline,
[51] 2-(3-(3-tolyl)-propiolyl)-1 ,2,3,4-tetrahydro-isoquinoline and [77] 2-(3-(3-cyano-phenyl)-propiolyl)-1,2,3,4-tetrahydro-isoquinoline;
in each case optionally in the form of a corresponding salt, or in each case optionally in the form of a corresponding solvate.

25. (canceled)

26. A method for producing a compound of the formula I according to claim 1, said method comprising: reacting at least one compound of the formula II, 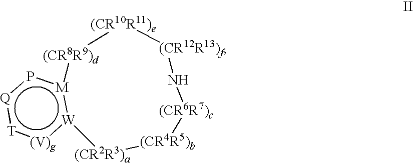
in which R², R³, R⁴, R⁵, R⁶, R⁷, R⁸, R⁹, R¹⁰, R¹¹, R¹², R¹³, M, W, P, Q, T, V, a, b, c, d, e, f and g have the meaning according to claim 1 with at least one compound of the formula R¹—C≡C—C(═O)—OH, in which R¹ has the meaning according to claim 1, optionally in a reaction medium, optionally in the presence of at least one suitable coupling agent, optionally in the presence of at least one base, or with at least one compound of the formula R¹—C≡C—C(═O)—X, in which R¹ has the above-stated meaning and X denotes a leaving group, in a reaction medium, optionally in the presence of at least one base, converting the compound into at least one corresponding compound of the formula I, optionally in the form of a corresponding salt, 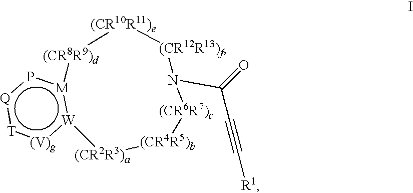
in which R¹, R², R³, R⁴, R⁵, R⁶, R⁷, R⁸, R⁹, R¹⁰, R¹¹, R¹², R¹³, M, W, P, Q, T, V, a, b, c, d, e, f and g have the above-stated meaning, and optionally purifying the compound or isolating the compound, or both;
or by reacting at least one compound of the formula II with propiolic acid HC≡C—C(═O)—OH] optionally in a reaction medium, optionally in the presence of at least one suitable coupling agent, optionally in the presence of at least one base, or by reaction with at least one compound of the formula HC≡C—C(═O)—X, in which X denotes a leaving group, in a reaction medium, optionally in the presence of at least one base, converting the compound into at least one corresponding compound of the formula III, optionally in the form of a corresponding salt, 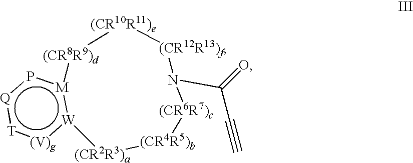
in which R², R³, R⁴, R⁵, R⁶, R⁷, R⁸, R⁹, R¹⁰, R¹¹, R¹², R¹³, M, W, P, Q, T, V, a, b, c, d, e, f and g have the above-stated meaning, and optionally purifying the compound or isolating the compound, or both,
and by reacting at least one compound of the formula III with at least one compound of the formula R¹—X, in which R¹ has the meaning according to claim 1 and X denotes a leaving group, optionally in the presence of at least one catalyst, optionally in the presence of at least one ligand, optionally in the presence of at least one inorganic salt, optionally in the presence of at least one copper salt, optionally in the presence of at least one organic or inorganic base, converting the compound into at least one corresponding compound of the formula I, optionally in the form of a corresponding salt, and optionally purifying the compound or isolating the compound, or both.

27. A pharmaceutical composition comprising: at least one substituted propiolic acid amide compound of the formula I 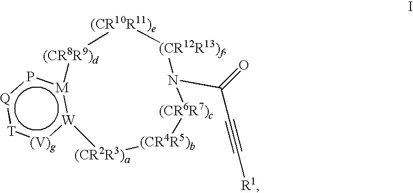
in which
R¹ denotes unsubstituted or substituted aryl or unsubstituted or substituted heteroaryl;
a, b, c, d, e and f in each case denote 0 or 1; wherein the sum of a, b, c, d, e and f is equal to 1, 2, 3, 4, 5 or 6;
R², R³, R⁴, R⁵, R⁶, R⁷, R⁸, R⁹, R¹⁰, R¹¹, R¹² and R¹³, mutually independently, in each case denote H; F; Cl; Br; I; —NO₂; —CN; —NH₂; —OH; —SH; —NH—R²²; —NR²³R²⁴; —O—R²⁵; —S—R²⁶; unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
or R² and R³ or R⁴ and R⁵ or R⁶ and R⁷ or R³ and R⁹ or R¹⁰ and R¹¹ or R¹² and R¹³ in each case together denote an oxo group (═O) or a thioxo group (═S);
M and W, mutually independently, in each case denote N or C;
P denotes CR¹⁴, N, NR¹⁵, O or S;
Q denotes CR¹⁶, N, NR¹⁷, O or S;
T denotes CR¹⁸, N, NR¹⁹, O or S;
V denotes CR²⁰, N, NR²¹, O or S;
g denotes 0 or 1:
wherein M, W; P, Q, T, V and g together form a 5- or 6-membered aromatic or heteroaromatic ring;
R¹⁴, R¹⁶, R¹⁸ and R²⁰, mutually independently, in each case denote H; F; Cl; Br; I; —NO₂; —CN; —NH₂; —OH; —SH; —C(═O)—OH; —C(═O)—H; —NH—C(═O)—H; —NH—R²²; —NR²³R²⁴; —O—R²⁵; —S—R²⁶; —C(═O)—R²⁷; —C(═O)—O—R²⁸; —O—C(═O)—R²⁹; —NR³¹—C(═O)—R³²; —C(═O)—NH₂; —C(═O)—NH—R³³; —C(═O)—NR³⁴R³⁵; —S(═O)—R³⁶; —S(═O)₂—R³⁷; —NH—C(═O)—NH—R³⁸; —NH—C(═S)—NH—R³⁹; —NH—S(═O)₂—R⁴⁰; —NR⁴¹—S(═O)₂—R⁴²; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
R¹⁵, R¹⁷, R¹⁹ and R²¹, mutually independently, in each case denote H; —C(═O)—OH; —C(═O)—H; —C(═O)—R²⁷; —C(═O)—O—R²⁸; —C(═O)—NH₂; —C(═O)—NH—R³³; —C(═O)—NR³⁴R³⁵; —S(═O)—R³⁶; —S(═O)₂—R³⁷; unsubstituted or at least monosubstituted alkyl, alkenyl or alkynyl; unsubstituted or at least monosubstituted heteroalkyl, heteroalkenyl or heteroalkynyl; unsubstituted or at least monosubstituted cycloalkyl or cycloalkenyl; unsubstituted or at least monosubstituted heterocycloalkyl or heterocycloalkenyl; unsubstituted or at least monosubstituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or at least monosubstituted -(heteroalkylene)-cycloalkyl, -(heteroalkenylene)-cycloalkyl, -(heteroalkylene)-cycloalkenyl or -(heteroalkenylene)-cycloalkenyl; unsubstituted or at least monosubstituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or at least monosubstituted -(heteroalkylene)-heterocycloalkyl, -(heteroalkenylene)-heterocycloalkyl, -(heteroalkylene)-heterocycloalkenyl or -(heteroalkenylene)-heterocycloalkenyl; unsubstituted or at least monosubstituted aryl; unsubstituted or at least monosubstituted heteroaryl; unsubstituted or at least monosubstituted -(alkylene)-aryl, -(alkenylene)-aryl, -(alkynylene)-aryl, -(heteroalkylene)-aryl or -(heteroalkenylene)-aryl; or unsubstituted or at least monosubstituted -(alkylene)-heteroaryl, -(alkenylene)-heteroaryl, -(alkynylene)-heteroaryl, -(heteroalkylene)-heteroaryl or -(heteroalkenylene)-heteroaryl;
and R²², R²³, R²⁴, R²⁵, R²⁶, R²⁷, R²⁸, R²⁹, R³¹, R³², R³³, R³⁴, R³⁵, R³⁶, R³⁷, R³⁸, R³⁹, R⁴⁰, R⁴¹ and R⁴², mutually independently, in each case denote unsubstituted or substituted alkyl, alkenyl or alkynyl; unsubstituted or substituted heteroalkyl, heteroalkenyl or heteroalkynyl; unsubstituted or substituted cycloalkyl or cycloalkenyl; unsubstituted or substituted heterocycloalkyl or heterocycloalkenyl; unsubstituted or substituted -(alkylene)-cycloalkyl, -(alkenylene)-cycloalkyl, -(alkynylene)-cycloalkyl, -(alkylene)-cycloalkenyl, -(alkenylene)-cycloalkenyl or -(alkynylene)-cycloalkenyl; unsubstituted or substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or substituted -(alkylene)-heterocycloalkyl, -(alkenylene)-heterocycloalkyl, -(alkynylene)-heterocycloalkyl, -(alkylene)-heterocycloalkenyl, -(alkenylene)-heterocycloalkenyl or -(alkynylene)-heterocycloalkenyl; unsubstituted or substituted -(heteroalkylene)-heterocycloalkyl, -(heteroalkenylene)-heterocycloalkyl, -(heteroalkylene)-heterocycloalkenyl; or -(heteroalkenylene)-heterocycloalkenyl; unsubstituted or substituted aryl; unsubstituted or substituted heteroaryl; unsubstituted or substituted -(alkylene)-aryl, -(alkenylene)-aryl, -(alkynylene)-aryl, -(heteroalkylene)-aryl or -(heteroalkenylene)-aryl; or unsubstituted or substituted -(alkylene)-heteroaryl, -(alkenylene)-heteroaryl, -(alkynylene)-heteroaryl, -(heteroalkylene)-heteroaryl or -(heteroalkenylene)-heteroaryl;
with the proviso that compounds are excluded in which g is 0, W and M in each case denote C, P denotes NR¹⁵, Q denotes CR¹⁶ and T denotes N;
and with the proviso that compounds are excluded in which g is 0, W and M in each case denote C, P denotes N, Q denotes CR¹⁶ and T denotes NR¹⁹;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate and optionally one or more physiologically acceptable auxiliary substances.

28. A pharmaceutical composition according to claim 27 comprising at least one compound of the formula Ie, 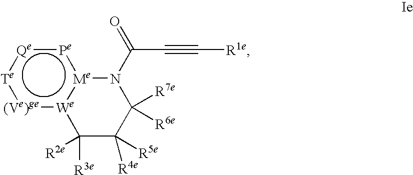
in which
R^{1e} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2e}, R^{3e}, R^{4e}, R^{5e}, R^{6e} and R^{7e}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
ge denotes 0 or 1;
W^{e}, M^{e}, P^{e}, Q^{e}, T^{e} and optionally V^{e} together denote a ring selected from the group consisting of 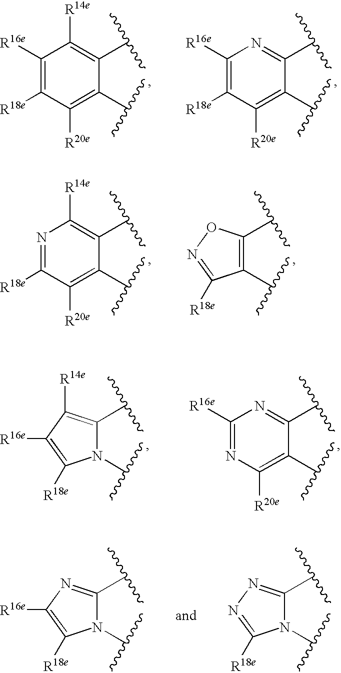
and R^{14e}, R^{16e}, R^{18e} and R^{20e}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

29. A pharmaceutical composition according to claim 27 comprising at least one compound of the formula Ie1, 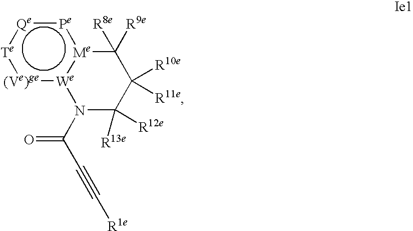
in which
R^{1e} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{8e}, R^{9e}, R^{10e}, R^{11e}, R^{12e} and R^{13e}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
ge denotes 0 or 1;
W^{e}, M^{e}, P^{e}, Q^{e}, T^{e} and optionally V^{e} together denote a ring selected from the group consisting of 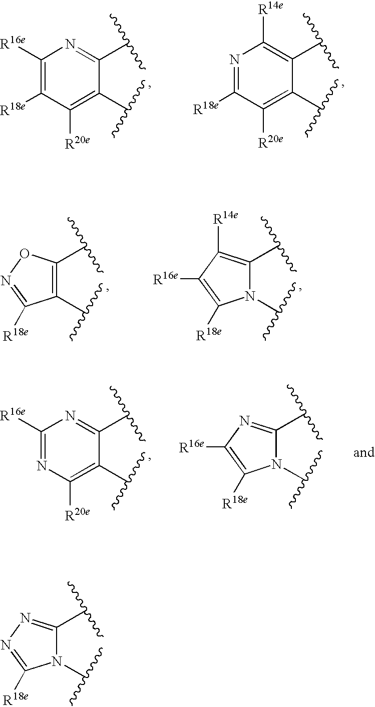
and R^{14e}, R^{16e}, R^{18e} and R^{20e}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

30. A pharmaceutical composition according to claim 27, wherein
R^{1e} denotes a residue selected from the group consisting of phenyl, thienyl and pyridinyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
R^{2e}, R^{3e}, R^{4e}, R^{5e}, R^{6e}, R^{7e}, R^{8e}, R^{9e}, R^{10e}, R^{11e}, R^{12e} and R^{13e}, mutually independently, in each case denote H; methyl or ethyl;
ge denotes 0 or 1;
W^{e}, M^{e}, P^{e}, Q^{e}, T^{e} and optionally V^{e} together denote a ring selected from the group consisting of 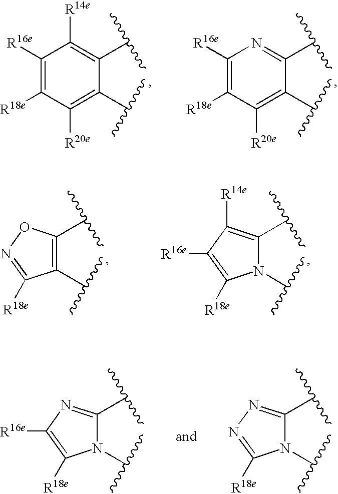
and R^{14e}, R^{16e}, R^{18e} and R^{20e}, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl.

31. A pharmaceutical composition according to claim 27 comprising at least one compound of the formula If, 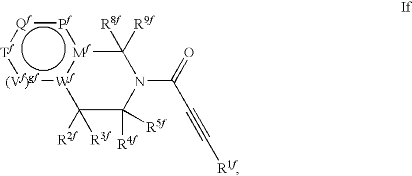
in which
R^{1f} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2f}, R^{3f}, R^{4f}, R^{5f}, R^{8f} and R^{9f}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
gf denotes 0 or 1;
W^{f}, M^{f}, P^{f}, Q^{f}, T^{f} and optionally V^{f} together denote a ring selected from the group consisting of 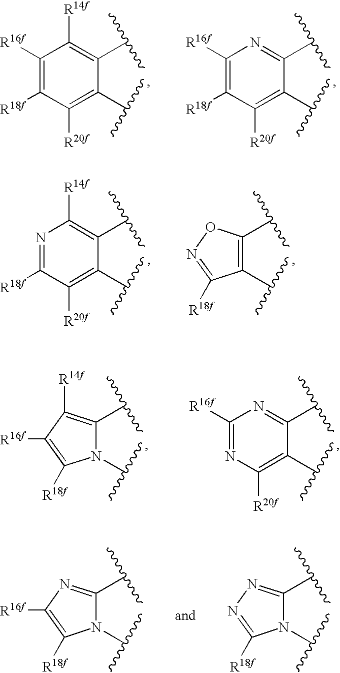
and R^{14f}, R¹⁶, R^{18f} and R²⁰, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

32. A pharmaceutical composition according to claim 27 comprising at least one compound of the formula If1, 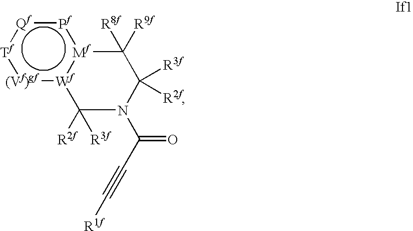
in which
R^{1f} denotes a residue selected from the group consisting of phenyl, pyridinyl and thienyl, which is in each case unsubstituted or substituted with 1 or 2 substituents mutually independently selected from the group consisting of F, Cl, Br, I, —CN, methyl, ethyl, n-propyl, isopropyl, n-butyl, 2-butyl, isobutyl, tert.-butyl, ethenyl, allyl, ethynyl, propynyl, cyclopropyl, —OH, —O—CH₃, —O—C₂H₅, —O—C₃H₇, —NO₂, —CF₃, —CH₂F, —CHF₂, —O—CF₃ and —S—CF₃;
R^{2f}, R^{3f}, R^{8f}, R^{9f}, R^{10f} and R^{11f}, mutually independently, in each case denote H; F; Cl; Br; I; —NO₂; —CN; —O—CH₃; —O—C₂H₅; —O—C(CH₃)₃; —O—CF₃; —O—CF₂H; —O—CFH₂; —O—C₂F₅; —O—CH₂—CF₃; —CF₃; —CF₂H; —CFH₂; —C₂F₅; —CH₂—CF₃; methyl; ethyl; n-propyl; isopropyl; n-butyl; cyclopropyl or cyclobutyl;
gf denotes 0 or 1;
W^{f}, M^{f}, P^{f}, Q^{f}, T^{f} and optionally V^{f} together denote a ring selected from the group consisting of 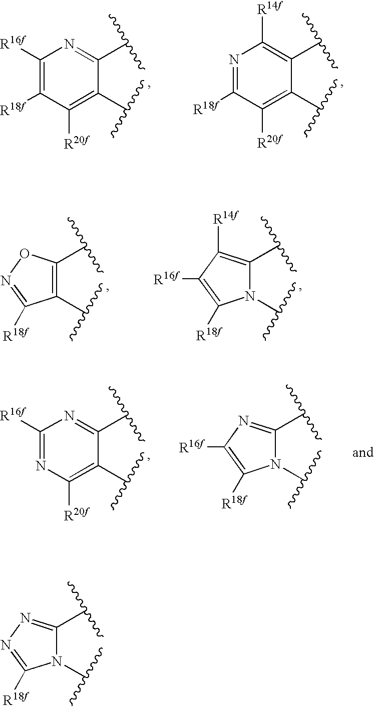
and R^{14f}, R^{16f}, R^{18f} and R^{20f}, mutually independently, in each case denote H; F; Cl; Br; I; —CF₃; —NO₂; —CN; —NH₂; —OH; —C(═O)—OH; —C(═O)—H; —NH—CH₃; —N(CH₃)₂; —O—CH₃; —O—C₂H₅; —O—C(CH₃)₃; —O—CF₃; —O—CF₂H; —O—CFH₂; —C(═O)—O—CH₃; —C(═O)—O—C₂H₅; —O—C(═O)—CH₃; —N(CH₃)—C(═O)—CH₃; —C(═O)—NH₂; —C(═O)—NH—CH₃; —C(═O)—NH—C₂H₅; methyl; ethyl; n-propyl; isopropyl; n-butyl; 2-butyl; isobutyl; tert.-butyl; or denote a residue selected from the group consisting of phenyl and benzyl, which is unsubstituted or substituted with 1, 2, 3, 4 or 5 substituents mutually independently selected from the group consisting of F, Cl, Br, I, —CN, —CF₃, methyl, ethyl, n-propyl, isopropyl, n-butyl, isobutyl, 2-butyl and tert.-butyl;
in each case optionally in the form of one of the pure stereoisomers thereof, in the form of one of the racemates thereof or in the form of a mixture of stereoisomers in any mixing ratio, or in each case in the form of a corresponding salt or in each case in the form of a corresponding solvate.

33. A pharmaceutical composition according to claim 31, wherein
R^{1f} denotes a residue selected from the group consisting of phenyl, thienyl and pyridinyl, which is in each case unsubstituted or substituted with 1 or 2 substituents mutually independently selected from the group consisting of methyl, ethyl, n-propyl, isopropyl, n-butyl, 2-butyl, isobutyl, tert.-butyl, —OH, —O—CH₃, —O—C₂H₅, —O—C₃H₇, —CF₃, —CN, F, Cl, Br and I;
R^{2f}, R^{3f}, R^{4f}, R^{5f}, R^{8f}, R^{9f}, R^{10e} and R^{11e}, mutually independently, in each case denote H; methyl or ethyl;
gf denotes 0 or 1;
W^{f}, M^{f}, P^{f}, Q^{f}, T^{f} and optionally V^{f} together denote a ring selected from the group consisting of 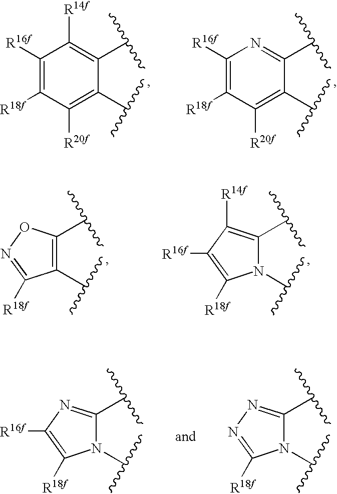
and R¹⁴, R^{16f}, R^{18f} and R^{20f}, mutually independently, in each case denote H; F; Cl; Br; —OH; —O—CH₃; —O—C₂H₅; —O—C(CH₃)₃; —O—CF₃; methyl; ethyl; n-propyl; isopropyl; n-butyl; 2-butyl; isobutyl; tert.-butyl or phenyl.

34. A pharmaceutical composition according to claim 27 comprising at least one compound selected from the group consisting of
[1] 2-(3-phenyl-propiolyl)-1,2,3,4-tetrahydro-isoquinoline,
[2] 1-(3-phenyl-propiolyl)-1,2,3,4-tetrahydro-quinoline,
[5] 2-(3-(3-chlorophenyl)-propiolyl)-1,2,3,4-tetrahydro-isoquinoline,
2-(3-pyrid-2-yl-propiolyl)-1,2,3,4-tetrahydro-isoquinoline,
[11] 2-(3-(3-chlorophenyl)-propiolyl)-6,7-dimethoxy-1,2,3,4-tetrahydro-isoquinoline,
[13] 2-(3-(3-chlorophenyl)-propiolyl)-6,7-dihydroxy-1,2,3,4-tetrahydro-isoquinoline,
[14] 6-methoxy-2-(3-phenyl-propiolyl)-1,2,3,4-tetrahydro-isoquinoline,
2-(3-(3-chlorophenyl)-propiolyl)-7-hydroxy-6-methoxy-1,2,3,4-tetrahydro-isoquinoline,
2-(3-(3-chlorophenyl)-propiolyl)-6-hydroxy-7-methoxy-1,2,3,4-tetrahydro-isoquinoline,
2-(3-(3-chlorophenyl)-propiolyl)-6-methoxy-1,2,3,4-tetrahydro-isoquinoline,
[18] 2-(3-(3-chlorophenyl)-propiolyl)-1-methyl-1,2,3,4-tetrahydro-isoquinoline,
2-(3-(3-chlorophenyl)-propiolyl)-3-methyl-1,2,3,4-tetrahydro-isoquinoline,
2-(3-(3-chlorophenyl)-propiolyl)-6-methyl-1,2,3,4-tetrahydro-isoquinoline,
2-(3-(3-chlorophenyl)-propiolyl)-7-methoxy-1,2,3,4-tetrahydro-isoquinoline,
[24] 2-(3-(3-chlorophenyl)-propiolyl)-8-methyl-1,2,3,4-tetrahydro-isoquinoline,
[25] 2-(3-(3-chlorophenyl)-propiolyl)-7-methyl-1,2,3,4-tetrahydro-isoquinoline,
[26] 2-(3-(3-chlorophenyl)-propiolyl)-5-methyl-1,2,3,4-tetrahydro-isoquinoline,
[28] 2-(3-(3-chlorophenyl)-propiolyl)-7-hydroxy-1,2,3,4-tetrahydro-isoquinoline,
[29] 2-(3-(3-chlorophenyl)-propiolyl)-8-methoxy-1,2,3,4-tetrahydro-isoquinoline,
[30] 2-(3-(3-chlorophenyl)-propiolyl)-5-methoxy-1,2,3,4-tetrahydro-isoquinoline,
[31] 2-(3-(3-chlorophenyl)-propiolyl)-1,8-dimethyl-1,2,3,4-tetrahydro-isoquinoline,
[32] 2-(3-(2,4-difluorophenyl)-propiolyl)-1,2,3,4-tetrahydro-isoquinoline,
[34] 2-(3-(3-methoxyphenyl)-propiolyl)-1,2,3,4-tetrahydro-isoquinoline,
[35] 2-(3-(4-fluoro-3-methylphenyl)-propiolyl)-1,2,3,4-tetrahydro-isoquinoline,
[37] 2-(3-(2-fluorophenyl)-propiolyl)-1,2,3,4-tetrahydro-isoquinoline,
[39] 2-(3-(4-tolyl)-propiolyl)-1,2,3,4-tetrahydro-isoquinoline,
[41] 2-(3-(4-trifluoromethylphenyl)-propiolyl)-1,2,3,4-tetrahydro-isoquinoline,
[43] 2-(3-phenyl-propiolyl)-1,2,3,4-tetrahydro-isoquinoline,
2-(3-(2,3-dimethylphenyl)-propiolyl)-1,2,3,4-tetrahydro-isoquinoline,
2-(3-(3,5-dimethylphenyl)-propiolyl)-1,2,3,4-tetrahydro-isoquinoline,
2-(3-(2,4-dichlorophenyl)-propiolyl)-1,2,3,4-tetrahydro-isoquinoline,
[51] 2-(3-(3-tolyl)-propiolyl)-1,2,3,4-tetrahydro-isoquinoline,
[53] 2-(3-(4-methoxyphenyl)-propiolyl)-1,2,3,4-tetrahydro-isoquinoline,
2-(3-(2-thienyl)-propiolyl)-1,2,3,4-tetrahydro-isoquinoline,
[57] 1-(3-(4-tolyl)-propiolyl)-1,2,3,4-tetrahydro-quinoline,
[58] 1-(3-(4-trifluoromethylphenyl)-propiolyl)-1,2,3,4-tetrahydro-quinoline,
[59] 1-(3-(2,3-dimethylphenyl)-propiolyl)-1,2,3,4-tetrahydro-quinoline,
[60] 1-(3-(2,4-dichlorophenyl)-propiolyl)-1,2,3,4-tetrahydro-quinoline,
[61] 1-(3-(2,4-dimethylphenyl)-propiolyl)-1,2,3,4-tetrahydro-quinoline,
[62] 1-(3-(4-tert.-butylphenyl)-propiolyl)-1,2,3,4-tetrahydro-quinoline,
[64] 2-(3-(4-fluorophenyl)-propiolyl)-1,2,3,4-tetrahydro-isoquinoline,
[67] 2-(3-(3-fluoro-4-methylphenyl)-propiolyl)-1,2,3,4-tetrahydro-isoquinoline,
[69] 2-(3-(4-tert.-butylphenyl)-propiolyl)-1,2,3,4-tetrahydro-isoquinoline,
[71] 2-(3-(3,4-dimethylphenyl)-propiolyl)-1,2,3,4-tetrahydro-isoquinoline,
[76] 2-(3-(2-cyano-phenyl)-propiolyl)-1,2,3,4-tetrahydro-isoquinoline,
[77] 2-(3-(3-cyano-phenyl)-propiolyl)-1,2,3,4-tetrahydro-isoquinoline,
[78] 1-(3-(2-tolyl)-propiolyl)-1,2,3,4-tetrahydro-quinoline and
[79] 1-(3-(2-trifluoromethylphenyl)-propiolyl)-1,2,3,4-tetrahydro-quinoline;
in each case optionally in the form of one of the pure stereoisomers thereof, in the form of one of the racemates thereof or in the form of a mixture of stereoisomers in any desired mixing ratio, or in each case in the form of a corresponding salts or in each case in the form of a corresponding solvate.

35. (canceled)

36. (canceled)

37. (canceled)

38. A method for mGluR5 receptor regulation comprising: administering an effective amount of at least one compound of claim 1 to a patient in need thereof.

39. A method for the prevention, or treatment, or simultaneous prevention and treatment of disorders, or diseases, or disorders and diseases which are at least partially mediated by mGluR5 receptors comprising: administering an effective amount of at least one compound of claim 1 to a patient in need thereof.

40. The method according to claim 39, wherein the disorders or diseases are selected from the group consisting of pain; migraine; depression; neurodegenerative diseases; cognitive diseases; anxiety states; panic attacks; epilepsy; coughing; urinary incontinence; diarrhoea; pruritus; schizophrenia; cerebral ischaemic episodes; muscle spasms; cramps; pulmonary diseases; regurgitation (vomiting); stroke; dyskinesia; retinopathy; lack of drive; laryngitis); disorders of food intake; dependency on alcohol; dependency on medicines; dependency on drugs; alcohol abuse; abuse of medicines; drug abuse; withdrawal symptoms associated with dependency on alcohol, medicines and/or drugs; development of tolerance towards medicines; gastro-oesophageal reflux syndrome; gastro-oesophageal reflux disease; irritable bowel syndrome; diuresis; antinatriuresis; disorders of the cardiovascular system; reduced vigilance; reduced libido; and disorders related to locomotor activity.

41. The method according to claim 40, wherein the disorder or disease is selected from the group consisting of pain selected from the group consisting of acute pain, chronic pain, neuropathic pain and visceral pain; anxiety states; panic attacks; dependency on alcohol; dependency on medicines; cognitive diseases; disorders of food intake from the group consisting of bulimia, cachexia, anorexia and obesity; dependency on nicotine- and/or cocaine; alcohol abuse; abuse of medicines; nicotine and/or cocaine abuse; withdrawal symptoms associated with dependency on alcohol, medicines and/or nicotine- and/or cocaine; development of tolerance towards natural or synthetic opioids; gastro-oesophageal reflux syndrome, gastro-oesophageal reflux disease and irritable bowel syndrome.

42. The method according to claim 40, wherein the disorder or disease is pain selected from the group consisting of acute pain, chronic pain, neuropathic pain and visceral pain.

43. The method according to claim 40, wherein the disorder or disease is selected from the group consisting of anxiety states and panic attacks.

44. A method of providing local anaesthesia to a patient in need thereof, said method comprising locally administering to said patient at least one compound according to claim 1.
